(19) United States
(12) Patent Application Publication
(10) Pub. No.: US 2024/0327495 A1
(43) Pub. Date: Oct. 3, 2024

(54) LIGAND-BINDING POLYPEPTIDES AND USES THEREOF

(71) Applicant: OXFORD UNIVERSITY INNOVATION LIMITED, Oxford, Oxfordshire (GB)

(72) Inventors: Mark HOWARTH, Oxford, Oxfordshire (GB); Niels WICKE, London, Greater London (GB)

(21) Appl. No.: 18/267,189

(22) PCT Filed: Dec. 15, 2021
(86) PCT No.: PCT/GB2021/053304

§ 371 Date: Jun. 14, 2023

(30) Foreign Application Priority Data
Dec. 15, 2020 (GB) 2019817.2

(51) Int. Cl. C07K 14/81; C12N 15/10; C40B 40/08

(52) U.S. Cl. CPC C07K 14/8114; C12N 15/1044; C40B 40/08; C07K 2319/31

(57) ABSTRACT

The present invention relates to polypeptides that are resistant to degradation in the gastrointestinal tract and that bind to a target (i.e. a target ligand). In particular, it provides a mutant Kunitz-type soybean trypsin inhibitor (SBTI) family polypeptide comprising two or more amino acid mutations compared to the corresponding unmutated (e.g. wild-type) SBTI family polypeptide, wherein the mutant SBTI family polypeptide comprises: (i) one or more amino acid mutations in a first domain corresponding to positions 22-25 of SEQ ID NO: 1; and (ii) one or more amino acid mutations in a second domain corresponding to positions 47-50 of SEQ ID NO: 1, wherein the mutant SBTI family polypeptide: (a) binds selectively to a ligand that does not bind to the corresponding unmutated (e.g. wild-type) SBTI family polypeptide; and (b) is resistant to cleavage by pepsin.

### FIELD OF THE INVENTION

The present invention relates generally to the field of protein-engineering. More particularly, the invention provides means for obtaining polypeptides that are resistant to degradation in the gastrointestinal tract and that bind to a target (i.e. a target ligand), particularly a target associated with the gastrointestinal tract, e.g. polypeptides that are suitable for oral administration. Specifically, the invention provides mutant Kunitz-type soybean trypsin inhibitor (SBTI) family polypeptides that bind selectively to a target ligand and means for obtaining the mutant polypeptides, e.g. nucleic acid and polypeptide libraries (e.g. phage display libraries) that may be used to identify mutant polypeptides that bind selectively to target ligands of interest. Such mutant polypeptides may have numerous biotechnology and medical utilities, particularly as therapeutics, diagnostics and nutraceuticals, e.g. orally-delivered therapeutics for the treatment of disorders and conditions of the gastrointestinal tract. The invention further provides nucleic acid molecules (e.g. vectors) encoding such mutant polypeptides.

### BACKGROUND TO THE INVENTION

The gastrointestinal (GI) tract is a series of organs specialized for digestion, generating an extremely hostile environment for proteins. In the mammalian stomach, proteins will encounter high concentrations of both hydrochloric acid (fasted median gastric pH 1.7) and pepsin. Therefore, the vast majority of proteins are rapidly denatured and hydrolyzed to peptides following entry to the stomach. Other proteases and bile acids will be encountered in the intestinal phase, making it even harder for ingested proteins to remain intact and functional. However, proteins are exceptionally powerful in their selective binding and catalysis, so there has been extensive effort to enable oral delivery of functional proteins.

Oral administration of therapeutic proteins is highly desirable compared to injections, to reduce the need for medical supervision and to improve patient compliance and quality of life. Orally administered drugs have the potential to reduce toxic side-effects for diseases of the GI tract. For example, intravenous administration of anti-TNFα antibodies is a leading treatment for inflammatory bowel disease (IBD) but is associated with an increased risk of opportunistic infections and cancer. In contrast, orally administered anti-TNFα biologics could be restricted to sites of damage and the intestinal lumen, thereby minimising systemic immunomodulation and the associated negative side-effects.

While monoclonal antibodies (mAbs) represent a relatively new class of promising drugs with potentially very specific effects, it is well-established that antibodies are rapidly digested and inactivated in the adult stomach. Accordingly, there have been extensive protein engineering efforts to develop alternative antibody formats or antibody-like protein scaffolds (e.g. nanobodies, DARPins, affibodies), with more robust characteristics. However, although these scaffolds show tremendous therapeutic and diagnostic potential, typically they have been optimized for performance at neutral pH and are also rapidly destroyed in the GI tract. While nanofitins (affitins) and nanobodies have been engineered to improve their resistance to degradative conditions encountered in the GI tract, further improvements are needed to provide ligand binding molecules suitable for oral administration.

Conventionally, modifications to improve the survival of proteins from degradation under stomach-like conditions have focussed on changes to the formulation of the proteins, e.g. administering the proteins in a large excess, with an additional component to neutralize acid and/or in a protected form, e.g. a tablet or capsule with a protective enteric coating.

Numerous spontaneous or pathogen-induced diseases are associated with the GI tract in humans and animals. For instance, Inflammatory Bowel Disease (IBD) and other GI inflammation disorders are increasingly common and new effective treatments are required, particularly a means for delivering and targeting drugs to the affected regions of the gut. In this respect, Crohn's disease affects any part of the gastrointestinal tract, from mouth to anus, although in the majority of the cases the disease starts in the distal small bowel. Ulcerative colitis is restricted to inflammation in the colon and involves only the mucosa. Common symptoms associated with IBD are abdominal pain, vomiting, diarrhoea, rectal bleeding, weight loss and cramps or spams in the lower abdomen. In severe cases, the tendency to develop intra-abdominal fistulas gives rise to deep infections.

Pathogenic diseases, such as Campylobacter jejuni infection in chickens and enterotoxigenic Escherichia coli (ETEC) infection of pigs, are significant sources of livestock loss and food-borne illness.

Notably, various enzymes (phytase, carbohydrases, proteases) have been extensively engineered for oral delivery and are in widespread use to improve animal growth and feed efficiency. However, some nutritional enzymes may be effective only when they are in the correct region of the GI tract. Thus, the efficacy of nutritional enzymes may benefit from targeting to sites of action or anchoring at these sites.

Accordingly, there is a need for new means for treating disorders of the GI tract, particularly molecules that target specific molecules and/or regions of the GI tract, i.e. for new molecules suitable for enteral, particularly oral, administration.

### SUMMARY OF THE INVENTION

The present inventors have unexpectedly determined that a protein scaffold particularly suited to the GI tract may be derived from the Kunitz-type soybean trypsin inhibitor (SBTI). Specifically, the inventors have determined that two closely positioned loops in SBTI can be mutated to create a recognition surface capable of binding selectively to a target of interest. Importantly and surprisingly, mutation of the loops, including the insertion of additional residues, did not affect the degradation-resistant properties of wild-type SBTI, which is stable in the presence of gastric concentrations of pepsin and at pH 2, and in the presence of intestinal bile acids and other proteases; conditions in which other protein scaffolds are rapidly digested. Advantageously, the inventors randomised the identified loops, including the insertion of additional residues, to generate a library of mutant polypeptides that could be screened, e.g. using phage display methods, to select mutant polypeptides (termed “gastrobodies”) that bind selectively to targets of interest. As discussed in the Examples, the domains of toxin A (TcdA) and toxin B (TcdB, e.g. GTD) from Clostridium difficile were selected as representative targets, since this organism is a leading cause of healthcare-associated infection.

Kunitz-type soybean trypsin inhibitor (SBTI) is the archetypal protease inhibitor from legume seeds, although numerous protease inhibitors from seeds and other sources are known. Thus, the choice to investigate SBTI as a potential scaffold for ligand binding domains represented a selection from a large number of potential starting points. It is thought that legume seed protease inhibitors have multiple endogenous roles, including in the defence against pathogens, regulating endogenous proteases, as storage proteins and to protect seed proteins during transit through the GI tract. In view of the multiple roles of these proteins, prior to the present invention, it was not clear whether modifications would be tolerated without affecting their resistance to GI conditions, particularly acid and pepsin.

While protease inhibitors found in legume seeds typically do not have high sequence similarity, many of these inhibitors share a common structure with SBTI, which has a β-trefoil type fold consisting of a closed barrel and a hairpin triplet, with internal pseudo-threefold symmetry. More specifically, the β-trefoil type fold found in protease inhibitors is composed of 12 beta-strands arranged in three similar units. Six of the beta-strands form an anti-parallel beta barrel around a central axis. The structure is analogous to a tree where the barrel forms the trunk and the loops joining the beta-strands are the branches/roots.

As most of the structural diversity between legume seed protease inhibitors is in the shape and size of the loops between the beta-strands, the inventors have further determined that the modifications to SBTI can be applied generally to other members of the Kunitz-type soybean trypsin inhibitor (SBTI) family. In particular, alignment of the structures of the other members of the Kunitz-type soybean trypsin inhibitor (SBTI) family with the structure of SBTI can be used to identify the loops that correspond to the loops suitable for mutation/randomisation in SBTI. While the corresponding loops in other members of the SBTI family may differ in size to the loops identified in SBTI, based on the experimental data provided herein, it is expected that mutation of these loops can be used to generate a recognition surface capable of binding selectively to a target of interest, without affecting other advantageous properties of these proteins, e.g. resistance to degradation in the GI tract. In this respect, the inventors have confirmed that several of the SBTI structural homologues identified herein have similar or better properties than SBTI with regard to the resistance to degradation in the GI tract.

Accordingly, in one aspect, the present invention provides a mutant Kunitz-type soybean trypsin inhibitor (SBTI) family polypeptide comprising two or more amino acid mutations compared to the corresponding unmutated (e.g. wild-type) SBTI family polypeptide, wherein the mutant SBTI family polypeptide comprises:
(i) one or more amino acid mutations in a first domain corresponding to positions 22-25 of SEQ ID NO: 1; and
(ii) one or more amino acid mutations in a second domain corresponding to positions 47-50 of SEQ ID NO: 1,
wherein the mutant SBTI family polypeptide:
(a) binds selectively to a ligand that does not bind to the corresponding unmutated (e.g. wild-type) SBTI family polypeptide; and
(b) is resistant to cleavage by pepsin.

In a further aspect, the invention provides a nucleic acid molecule encoding the mutant SBTI family polypeptide of the invention.

The present invention also provides a pharmaceutical composition comprising the mutant SBTI family polypeptide of the invention, optionally wherein the pharmaceutical composition is formulated for oral administration.

The invention further provides a mutant SBTI family polypeptide of the invention for use in therapy or diagnosis.

Alternatively viewed, the invention provides a method of treating or diagnosing a disease or condition in a subject, the method comprising administering a mutant SBTI family polypeptide of the invention or a pharmaceutical composition of the invention to a subject in need thereof.

In a further aspect, the invention provides the use of a mutant SBTI family polypeptide of the invention in the manufacture or preparation of a medicament for treating or diagnosing a disease or condition in a subject.

The present invention also provides the use of a nucleic acid molecule encoding a Kunitz-type soybean trypsin inhibitor (SBTI) family polypeptide as a starting molecule in a mutation and selection screening process for obtaining a mutant SBTI family polypeptide comprising two or more amino acid mutations compared to the corresponding unmutated (e.g. wild-type) SBTI family polypeptide, wherein the mutant SBTI family polypeptide comprises:
(i) one or more amino acid mutations in a first domain corresponding to positions 22-25 of SEQ ID NO: 1; and
(ii) one or more amino acid mutations in a second domain corresponding to positions 47-50 of SEQ ID NO: 1,
and wherein the mutant SBTI family polypeptide:
(a) binds selectively to a ligand that does not bind to the corresponding unmutated (e.g. wild-type) SBTI family polypeptide; and
(b) is resistant to cleavage by pepsin.

In another aspect, the invention provides a library of nucleic acid molecules encoding a plurality of mutant SBTI family polypeptides each comprising two or more amino acid mutations compared to their corresponding unmutated (e.g. wild-type) SBTI family polypeptides, wherein each mutant SBTI family polypeptide comprises:
(i) one or more amino acid mutations in a first domain corresponding to positions 22-25 of SEQ ID NO: 1; and
(ii) one or more amino acid mutations in a second domain corresponding to positions 47-50 of SEQ ID NO: 1.

The invention further provides a plurality of mutant SBTI family polypeptides encoded by the library of nucleic acid molecules of the invention.

The invention also provides the use of the library of nucleic acid molecules of the invention or the plurality of mutant SBTI family polypeptides of the invention in a screening method to identify a mutant SBTI family polypeptide that binds selectively to a ligand that does not bind to the corresponding unmutated (e.g. wild-type) SBTI family polypeptide.

In yet another aspect, the invention provides the use of the plurality of mutant SBTI family polypeptides of the invention to:
(i) identify a mutant SBTI family polypeptide that binds selectively to a region of interest of the gastrointestinal tract of an animal; and/or
(ii) identify a ligand in the gastrointestinal tract.

In a further aspect, the invention provides a method of identifying a mutant SBTI family polypeptide that binds selectively to a ligand of interest (e.g. a ligand that does not bind to the corresponding unmutated (e.g. wild-type) SBTI family polypeptide) comprising:
(i) providing a plurality of mutant SBTI family polypeptides of the invention;
(ii) contacting the plurality of mutant SBTI family polypeptides of (i) with the ligand of interest;
(iii) isolating a mutant SBTI family polypeptide that binds selectively to the ligand of interest thereby identifying a mutant SBTI family polypeptide that binds selectively to the ligand of interest.

In another aspect, the invention provides a method of identifying a mutant SBTI family polypeptide that binds selectively to a region of interest of the gastrointestinal tract of an animal comprising:
(i) administering a plurality of mutant SBTI family polypeptides of the invention to the gastrointestinal tract of an animal (e.g. orally);
(ii) isolating a mutant SBTI family polypeptide (e.g. a phage particle displaying a mutant SBTI family polypeptide) that is non-covalently bound to the region of interest of gastrointestinal tract of the animal; and
(iii) identifying the mutant SBTI family polypeptide isolated in step (ii).

### DETAILED DESCRIPTION

The terms “Kunitz-type soybean trypsin inhibitor family polypeptide” and “SBTI family polypeptide” are used interchangeably herein and refer to a member of the Kunitz-type soybean trypsin inhibitor superfamily (InterPro classification IPR011065), which consists primarily of proteinase inhibitors of leguminosae (Fabaceae) seeds.

SBTI family polypeptides that may find particular utility in the invention share structural homology to Kunitz-type soybean trypsin inhibitor (SBTI, Uniprot ID P01070, e.g. SEQ ID NO: 1). Structural homology may be determined by comparing the known or predicted structure of an SBTI family polypeptide with SBTI using any suitable method known in the art, e.g. using PyMOL. Suitably, a SBTI family polypeptide for use in the invention comprises a β-trefoil type fold composed of 12 beta-strands arranged in three similar units as described above. SBTI family polypeptides of particular utility in the invention include polypeptides in the MEROPS Family I3, clan IC (InterPro classification IPR002160).

An SBTI family polypeptide typically contains at least one disulfide bond and may contain two or three disulfide bonds. In some embodiments, an SBTI family polypeptide contains at least two disulfide bonds. As discussed further below, the cysteine residues involved in the formation of the disulfide bonds are conserved (i.e. not mutated) in the mutant SBTI family polypeptide of the invention. However, cysteine residues that are involved in the formation of the disulfide bonds may be mutated (e.g. substituted) in the mutant SBTI family polypeptide of the invention.

Typically, an unmutated SBTI family polypeptide for use in the present invention is not a glycosylated polypeptide, i.e. it does not contain any glycosylation sites or motifs.

As noted above, most SBTI family polypeptides are protease inhibitors. In particular, an SBTI family polypeptide for use in the invention may be a serine protease inhibitor, preferably a trypsin and/or chymotrypsin inhibitor. However, the WBA (Winged bean albumin) protein (Uniprot ID P15465, e.g. SEQ ID NO: 2), which is a close structural homologue of SBTI, does not have any known proteinase inhibitory activity. Thus, it is not essential that the SBTI family polypeptide is a protease inhibitor, although this is preferred in some embodiments.

SBTI family polypeptides that may find particular utility in the invention include SBTI (Soybean trypsin inhibitor, Uniprot ID P01070, e.g. SEQ ID NO: 1, 12, 13 or 62), WBA (Winged bean albumin, Uniprot ID P15465, e.g. SEQ ID NO: 2), ECTI (Erythrina caffra trypsin inhibitor DE-3, Uniprot ID P09943, e.g. SEQ ID NO: 3), WCI (Winged bean chymotrypsin inhibitor 3, Uniprot ID P10822, e.g. SEQ ID NO: 4), CATI (Cicer arietinum trypsin inhibitor 2, Uniprot ID Q9M3Z7, e.g. SEQ ID NO: 5), EnCTI (Enterolobium contortisiliquum trypsin inhibitor, Uniprot ID P86451, e.g. SEQ ID NO: 6), DRTI (Delonix regia trypsin inhibitor, Uniprot ID P83667, e.g. SEQ ID NO: 7), SOTI (Senna obtusifolia trypsin inhibitor 1, Uniprot ID A0A097P6E1, e.g. SEQ ID NO: 8), BBTI (Bauhinia bauhinioides trypsin inhibitor, Uniprot ID Q6VEQ7 also known as Bauhinia bauhinioides Kunitz-type serine protease inhibitor (BBKI), Uniprot ID P83052, e.g. SEQ ID NO: 9 or 85), AMTI (Alocasia macrorrhiza trypsin/chymotrypsin inhibitor, Uniprot ID P35812, e.g. SEQ ID NO: 10) and SSTI (Sagittaria sagittifolia trypsin inhibitor, Uniprot ID Q7M1P4, e.g. SEQ ID NO: 11) or functionally and/or structurally-equivalent variants thereof, particularly natural biological variants (e.g. allelic variants or geographical variants within a species or alternatively in different genera or families). Thus, in some embodiments, the natural biological variants are variants within the Fabaceae family (also known as the leguminosae family).

The Uniprot accession numbers recited herein refer to the full-length amino acid sequences of the polypeptides, i.e. containing the signal peptide and/or propeptide. However, the mutated SBTI family polypeptides of the invention typically are based on mature sequences, i.e. without the signal peptide and/or propeptide. In this respect, the amino acid sequences referenced in the SEQ ID NOs. above refer to mature sequences. Accordingly, an unmutated SBTI family polypeptide may comprise or consist of an amino acid sequence defined above.

Functionally- and/or structurally-equivalent SBTI family polypeptides include polypeptides that are related to, or derived from, a naturally-occurring protein. Functionally- and/or structurally-equivalent SBTI family polypeptides may be obtained by modifying a native amino acid sequence by single or multiple (e.g. 2-20, preferably 2-10) amino acid mutations (i.e. substitutions, additions and/or deletions), but without destroying the molecule's function and/or overall structure. As a representative example, a functionally-equivalent protein may contain one or more amino acid mutations that do not eliminate the protease inhibitory activity of the molecule (e.g. the functionally-equivalent variant has at least 50%, preferably at least 70%, 80% or 90% of the protease inhibitory activity of the related protein). Similarly, a structurally-equivalent variant may contain one or more amino acid mutations that do not disrupt the overall structure of the protein, e.g. the β-trefoil fold. Notably, a structurally-equivalent variant may contain a mutation that eliminates the protease inhibitory activity. Preferably mutations in a functionally-equivalent variants do not disrupt the overall structure of the protein, e.g. the β-trefoil fold.

Thus, the term “unmutated SBTI family polypeptide” typically refers to a polypeptide that occurs in nature, i.e. a native or wild-type polypeptide. As noted above, this includes natural biological variants, i.e. isoforms. Moreover, an SBTI family polypeptide may be modified without affecting its structure, as described above. For instance, an SBTI family polypeptide may be modified to eliminate its protease inhibitory activity or to remove or introduce a cysteine residue, e.g. modified outside of the domains identified herein. It will be evident that such modified polypeptides (or their encoding nucleic acid molecules) would be suitable as a starting molecule for a mutation and selection screening process as described herein. Thus, in some embodiments, an unmutated SBTI family polypeptide may include a modified polypeptide, i.e. containing one or more mutations outside the domains specified herein. However, in preferred embodiments, an unmutated SBTI family polypeptide refers to a native or wild-type polypeptide.

As a representative example, isoforms of SBTI include amino acid sequences as set forth in SEQ ID NOs: 12 and 13, which contain 8 and 1 amino acid substitutions relative to SEQ ID NO: 1, respectively. A further isoform of SBTI contains a C-terminal tail, as shown in SEQ ID NO: 62.

Accordingly, in some embodiments, an unmutated SBTI family polypeptide may be selected from:
(i) a polypeptide comprising an amino acid sequence as set forth in SEQ ID NO: 1, 12, 13 or 62 (e.g. SBTI, Uniprot ID P01070);
(ii) a polypeptide comprising an amino acid sequence as set forth in SEQ ID NO: 2 (e.g. WBA, Uniprot ID P15465);
(iii) a polypeptide comprising an amino acid sequence as set forth in SEQ ID NO: 3 (e.g. ECTI, Uniprot ID P09943);
(iv) a polypeptide comprising an amino acid sequence as set forth in SEQ ID NO: 4 (e.g. WCI, Uniprot ID P10822);
(v) a polypeptide comprising an amino acid sequence as set forth in SEQ ID NO: 5 (e.g. CATI, Uniprot ID Q9M3Z7);
(vi) a polypeptide comprising an amino acid sequence as set forth in SEQ ID NO: 6 (e.g. EnCTI, Uniprot ID P86451);
(vii) a polypeptide comprising an amino acid sequence as set forth in SEQ ID NO: 7 (e.g. DRTI, Uniprot ID P83667);
(viii) a polypeptide comprising an amino acid sequence as set forth in SEQ ID NO: 8 (e.g. SOTI, Uniprot ID A0A097P6E1);
(ix) a polypeptide comprising an amino acid sequence as set forth in SEQ ID NO: 9 (e.g. BBTI/BBKI, Uniprot ID Q6VEQ7 and Uniprot ID P83052) or SEQ ID NO: 85;
(x) a polypeptide comprising an amino acid sequence as set forth in SEQ ID NO: 10 (e.g. AMTI, Uniprot ID P35812);
(xi) a polypeptide comprising an amino acid sequence as set forth in SEQ ID NO: 11 (e.g. SSTI, Uniprot ID Q7M1P4); or
(xii) a polypeptide comprising an amino acid sequence having at least 80% (e.g. 85%, 90% or 95%) sequence identity to an amino acid sequence of any one of SEQ ID NOs: 1-13, 62 or 85, preferably wherein the polypeptide is a wild-type polypeptide.

In some embodiments, an unmutated SBTI family polypeptide may be selected from:
(i) a polypeptide comprising an amino acid sequence as set forth in SEQ ID NO: 1, 12, 13 or 62 (e.g. SBTI, Uniprot ID P01070);
(ii) a polypeptide comprising an amino acid sequence as set forth in SEQ ID NO: 3 (e.g. ECTI, Uniprot ID P09943);
(iii) a polypeptide comprising an amino acid sequence as set forth in SEQ ID NO: 4 (e.g. WCI, Uniprot ID P10822);
(iv) a polypeptide comprising an amino acid sequence as set forth in SEQ ID NO: 9 (e.g. BBTI/BBKI, Uniprot ID Q6VEQ7 and Uniprot ID P83052) or SEQ ID NO: 85; or
(v) a polypeptide comprising an amino acid sequence having at least 80% (e.g. 85%, 90% or 95%) sequence identity to an amino acid sequence of any one of SEQ ID NOs: 1, 3, 4, 9, 12, 13, 62 or 85, preferably wherein the polypeptide is a wild-type polypeptide.

Unmutated SBTI family polypeptides and mutated SBTI family polypeptides of the invention typically refer to mature sequences, i.e. without the signal peptide and/or propeptide. For instance, a mature unmutated SBTI family polypeptide typically will have protease inhibitor activity. Accordingly, an unmutated SBTI family polypeptide may comprise or consist of an amino acid sequence defined above.

The term “isoform” herein refers to a protein that is a member of a group of similar proteins (e.g. with at least 80%, such as at least 85%, 90% or 95%) that expressed from a single gene or a gene family.

A “mutant SBTI family polypeptide” (also referred to herein as a “mutant polypeptide”) refers to a polypeptide that contains two or more amino acid mutations compared to the corresponding unmutated (e.g. wild-type) SBTI family polypeptide. In particular, a mutant SBTI family polypeptide contains at least one amino acid mutation in a first domain corresponding to positions 22-25 of SEQ ID NO: 1 and at least one amino acid mutation in a second domain corresponding to positions 47-50 of SEQ ID NO: 1.

A “corresponding unmutated (e.g. wild-type) SBTI family polypeptide” refers to polypeptide from which the mutant SBTI family polypeptide is derived, e.g. the unmutated (e.g. wild-type) SBTI family polypeptide which has the highest level of sequence identity to the mutant SBTI family polypeptide. As discussed in detail below, the mutant SBTI family polypeptide of the invention may be obtained by screening a plurality of polypeptides containing each containing at least one mutation in each of the domains specified above. The plurality of mutant polypeptides is encoded by a library of nucleic acid molecules (e.g. encoding a phage display library) which have been mutated relative to a starting molecule, i.e. a nucleic acid molecule encoding an unmutated SBTI family polypeptide. Thus, a corresponding unmutated (e.g. wild-type) SBTI family polypeptide may refer to a polypeptide encoded by the nucleic acid starting molecule used to generate the library of nucleic acid molecules from which the mutated SBTI family polypeptide was obtained.

As a representative example, for a mutant SBTI family polypeptide obtained by screening a plurality of polypeptides encoded by a library of nucleic acid molecules that were generated using a nucleic acid molecule encoding SBTI (e.g. encoding SEQ ID NO: 1) as a starting molecule, the corresponding unmutated SBTI family polypeptide will be SBTI (e.g. SEQ ID NO: 1 or an isoform thereof, e.g. SEQ ID NO: 12, 13 or 62).

As noted above, SBTI family polypeptides do not share high levels of sequence similarity. Thus, an unmutated (e.g. wild-type) SBTI family polypeptide which has at least 70% (e.g. at least 75%, 80% or 85%) sequence identity to the mutant SBTI family polypeptide may be viewed as the corresponding unmutated (e.g. wild-type) SBTI family polypeptide. The skilled person readily could determine which unmutated (e.g. wild-type) SBTI family polypeptide corresponds to the mutant SBTI family polypeptide (e.g. based on sequence identity) using routine methods known in the art, e.g. sequence alignment methods.

Sequence identity may be determined by any suitable means known in the art, e.g. using the SWISS-PROT protein sequence databank using FASTA pep-cmp with a variable pamfactor, and gap creation penalty set at 12.0 and gap extension penalty set at 4.0, and a window of 2 amino acids. Other programs for determining amino acid sequence identity include the BestFit program of the Genetics Computer Group (GCG) Version 10 Software package from the University of Wisconsin. The program uses the local homology algorithm of Smith and Waterman with the default values: Gap creation penalty-8, Gap extension penalty=2, Average match=2.912, Average mismatch=−2.003.

Preferably said comparison is made over the full length of the sequence, but may be made over a smaller window of comparison, e.g. less than 100, 80 or 50 contiguous amino acids.

A mutant SBTI family polypeptide contains at least one amino acid mutation in two domains, which correspond to positions 22-25 of SEQ ID NO: 1 and positions 47-50 of SEQ ID NO: 1. As noted above, SBTI family polypeptides share a common structural homology and this can be used to determine which domains correspond to (i.e. are equivalent to) the domains specified above with respect to SBTI (e.g. SEQ ID NO: 1).

In this respect, SBTI family polypeptides contain 12 beta-strands arranged in three similar units. Six of the beta-strands form an anti-parallel beta barrel around a central axis. Table 1 below identifies the positions of each beta-strand in SEQ ID NO: 1, which are numbered consecutively from N-terminus to C-terminus. Notably, beta-strands 1, 4, 5, 8, 9 and 12 form the anti-parallel beta-barrel.

TABLE 1
Beta-strand positions and residues
in SBTI (SEQ ID NO: 1)

| Beta- | Residue | | SEQ ID |
|---|---|---|---|
| strand # | # | Residues | NO: |
| 1 | 15-20 | GTYYIL | 14 |
| 2 | 29-32 | IRAA | 15 |
| 3 | 42-45 | TVVQ | 16 |
| 4 | 56-59 | TIIS | 17 |
| 5 | 73-77 | LSLKF | 18 |
| 6 | 94-96 | SVV | 19 |
| 7 | 104-106 | AVK | 20 |
| 8 | 116-122 | GWFRLER | 21 |
| 9 | 131-137 | YKLVFCP | 22 |
| 10 | 148-152 | IGISI | 23 |
| 11 | 159-163 | RRLVV | 24 |
| 12 | 171-175 | VQFQK | 25 |

Thus, the first domain (which corresponds to positions 22-25 of SEQ ID NO: 1) is located between beta-strands 1 and 2. The second domain (which corresponds to positions 47-50 of SEQ ID NO: 1) is located between beta-strands 3 and 4.

Accordingly, first and second domain corresponding to the positions set out above (i.e. a domain at an equivalent position) in an SBTI family polypeptide refers to domains between beta-strands 1 and 2 and beta-strands 3 and 4, respectively. In particular, the domains defined above with respect to SEQ ID NO: 1, refer to loops between the beta-strands. The skilled person readily could determine which residues in an SBTI family polypeptide correspond to the residues in the first and second domains by comparing (e.g. aligning) the predicted or known structure (e.g. from the protein data bank (PDB)) of the SBTI family polypeptide with the structure of SBTI (e.g. SEQ ID NO: 1) (e.g. using PyMOL) and identifying the residues in the loops in the SBTI family polypeptide between beta-strands 1 and 2 and beta-strands 3 and 4.

As most of the structural diversity between SBTI family polypeptides is found in the shape and size of the loops between the beta-strands, it will be evident that size of the first and second domains in an SBTI family polypeptide may differ from the size of the first and second domains in SBTI. For instance, the first domain may contain between 2-8 amino acids, such as 3-6 amino acids, typically 3, 4, or 5 amino acids. Similarly, the second domain may contain between 2-6 amino acids, such as 2-5 or 2-4 amino acids, typically 3 or 4 amino acids.

Table 2 below sets out the positions of the first and second domains corresponding to the domains in SEQ ID NO: 1 for SEQ ID NOs: 2-11.

TABLE 2
Positions of the first and second domains in SEQ ID NOs: 2-11

| | SEQ | | Residue | Residue |
|---|---|---|---|---|
| | ID | PDB | numbers of | numbers of |
| Protein | NO: | ID | first domain | second domain |
| WBA | 2 | 1WBA | 24-26 | 50-51 |
| ECTI | 3 | 1TIE | 21-25 | 47-50 |
| WCI | 4 | 2BEA | 23-27 | 49-52 |
| CATI | 5 | 5XOZ | 28-33 | 55-58 |
| EnCTI | 6 | 4J2K | 22-26 | 48-51 |
| DRTI | 7 | 1R8N | 25-30 | 52-55 |
| SOTI | 8 | 6KV2 | 21-23 | 48-49 |
| BBTI/BBKI | 9 | 6DWH/4ZOT | 24-27 | 49-51 |
| AMTI | 10 | 5YCZ | 23-26 | 47-49 |
| SSTI | 11 | 3E8L | 26-30 | 50-52 |

Thus, in some embodiments, the invention provides a mutant SBTI polypeptide (e.g. a mutant of a polypeptide comprising an amino acid sequence as set forth in SEQ ID NO: 1 or a variant thereof, such as a polypeptide having at least 80% sequence identity to SEQ ID NO: 1, e.g. an isoform of SBTI, such as SEQ ID NO: 12, 13 or 62) comprising two or more amino acid mutations compared to the corresponding unmutated polypeptide (i.e. SEQ ID NO: 1 or variant thereof, e.g. SEQ ID NO: 12, 13 or 62), wherein the mutant SBTI polypeptide comprises:
(i) one or more amino acid mutations in positions equivalent to positions 22-25 of SEQ ID NO: 1; and
(ii) one or more amino acid mutations in positions equivalent to positions 47-50 of SEQ ID NO: 1,
wherein the mutant SBTI polypeptide:
(a) binds selectively to a ligand that does not bind to the corresponding unmutated polypeptide; and
(b) is resistant to cleavage by pepsin.

In another aspect, the invention provides a mutant WBA polypeptide (e.g. a mutant of a polypeptide comprising an amino acid sequence as set forth in SEQ ID NO: 2 or a variant thereof, such as a polypeptide having at least 80% sequence identity to SEQ ID NO: 2, e.g. an isoform of WBA) comprising two or more amino acid mutations compared to the corresponding unmutated polypeptide (i.e. SEQ ID NO: 2 or variant thereof), wherein the mutant WBA polypeptide comprises:
(i) one or more amino acid mutations in positions equivalent to positions 24-26 of SEQ ID NO: 2; and
(ii) one or more amino acid mutations in positions equivalent to positions 50-51 of SEQ ID NO: 2,
wherein the mutant WBA polypeptide:
(a) binds selectively to a ligand that does not bind to the corresponding unmutated polypeptide; and
(b) is resistant to cleavage by pepsin.

In another aspect, the invention provides a mutant ECTI polypeptide (e.g. a mutant of a polypeptide comprising an amino acid sequence as set forth in SEQ ID NO: 3 or a variant thereof, such as a polypeptide having at least 80% sequence identity to SEQ ID NO: 3, e.g. an isoform of ECTI) comprising two or more amino acid mutations compared to the corresponding unmutated polypeptide (i.e. SEQ ID NO: 3 or variant thereof), wherein the mutant ECTI polypeptide comprises:
(i) one or more amino acid mutations in positions equivalent to positions 21-25 of SEQ ID NO: 3; and
(ii) one or more amino acid mutations in positions equivalent to positions 52-55 of SEQ ID NO: 3,
wherein the mutant ECTI polypeptide:
(a) binds selectively to a ligand that does not bind to the corresponding unmutated polypeptide; and
(b) is resistant to cleavage by pepsin.

In another aspect, the invention provides a mutant WCI polypeptide (e.g. a mutant of a polypeptide comprising an amino acid sequence as set forth in SEQ ID NO: 4 or a variant thereof, such as a polypeptide having at least 80% sequence identity to SEQ ID NO: 4, e.g. an isoform of WCI) comprising two or more amino acid mutations compared to the corresponding unmutated polypeptide (i.e. SEQ ID NO: 4 or variant thereof), wherein the mutant WCI polypeptide comprises:
(i) one or more amino acid mutations in positions equivalent to positions 23-27 of SEQ ID NO: 4; and
(ii) one or more amino acid mutations in positions equivalent to positions 49-52 of SEQ ID NO: 4,
wherein the mutant WCI polypeptide:
(a) binds selectively to a ligand that does not bind to the corresponding unmutated polypeptide; and
(b) is resistant to cleavage by pepsin.

In another aspect, the invention provides a mutant CATI polypeptide (e.g. a mutant of a polypeptide comprising an amino acid sequence as set forth in SEQ ID NO: 5 or a variant thereof, such as a polypeptide having at least 80% sequence identity to SEQ ID NO: 5, e.g. an isoform of CATI) comprising two or more amino acid mutations compared to the corresponding unmutated polypeptide (i.e. SEQ ID NO: 5 or variant thereof), wherein the mutant CATI polypeptide comprises:
(i) one or more amino acid mutations in positions equivalent to positions 28-33 of SEQ ID NO: 5; and
(ii) one or more amino acid mutations in positions equivalent to positions 55-58 of SEQ ID NO: 5,
wherein the mutant CATI polypeptide:
(a) binds selectively to a ligand that does not bind to the corresponding unmutated polypeptide; and
(b) is resistant to cleavage by pepsin.

In another aspect, the invention provides a mutant EnCTI polypeptide (e.g. a mutant of a polypeptide comprising an amino acid sequence as set forth in SEQ ID NO: 6 or a variant thereof, such as a polypeptide having at least 80% sequence identity to SEQ ID NO: 6, e.g. an isoform of EnCTI) comprising two or more amino acid mutations compared to the corresponding unmutated polypeptide (i.e. SEQ ID NO: 6 or variant thereof), wherein the mutant EnCTI polypeptide comprises:
(i) one or more amino acid mutations in positions equivalent to positions 22-26 of SEQ ID NO: 6; and
(ii) one or more amino acid mutations in positions equivalent to positions 48-51 of SEQ ID NO: 6,
wherein the mutant EnCTI polypeptide:
(a) binds selectively to a ligand that does not bind to the corresponding unmutated polypeptide; and
(b) is resistant to cleavage by pepsin.

In another aspect, the invention provides a mutant DRTI polypeptide (e.g. a mutant of a polypeptide comprising an amino acid sequence as set forth in SEQ ID NO: 7 or a variant thereof, such as a polypeptide having at least 80% sequence identity to SEQ ID NO: 7, e.g. an isoform of DRTI) comprising two or more amino acid mutations compared to the corresponding unmutated polypeptide (i.e. SEQ ID NO: 7 or variant thereof), wherein the mutant DRTI polypeptide comprises:
(i) one or more amino acid mutations in positions equivalent to positions 25-30 of SEQ ID NO: 7; and
(ii) one or more amino acid mutations in positions equivalent to positions 52-55 of SEQ ID NO: 7,
wherein the mutant DRTI polypeptide:
(a) binds selectively to a ligand that does not bind to the corresponding unmutated polypeptide; and
(b) is resistant to cleavage by pepsin.

In another aspect, the invention provides a mutant SOTI polypeptide (e.g. a mutant of a polypeptide comprising an amino acid sequence as set forth in SEQ ID NO: 8 or a variant thereof, such as a polypeptide having at least 80% sequence identity to SEQ ID NO: 8, e.g. an isoform of SOTI) comprising two or more amino acid mutations compared to the corresponding unmutated polypeptide (i.e. SEQ ID NO: 8 or variant thereof), wherein the mutant SOTI polypeptide comprises:
(i) one or more amino acid mutations in positions equivalent to positions 21-23 of SEQ ID NO: 8; and
(ii) one or more amino acid mutations in positions equivalent to positions 48-49 of SEQ ID NO: 8,
wherein the mutant SOTI polypeptide:
(a) binds selectively to a ligand that does not bind to the corresponding unmutated polypeptide; and
(b) is resistant to cleavage by pepsin.

In another aspect, the invention provides a mutant BBTI (BBKI) polypeptide (e.g. a mutant of a polypeptide comprising an amino acid sequence as set forth in SEQ ID NO: 9 or 85 or a variant thereof, such as a polypeptide having at least 80% sequence identity to SEQ ID NO: 9 or 85, e.g. an isoform of BBTI (BBKI)) comprising two or more amino acid mutations compared to the corresponding unmutated polypeptide (i.e. SEQ ID NO: 9 or 85 or variant thereof), wherein the mutant BBTI (BBKI) polypeptide comprises:
(i) one or more amino acid mutations in positions equivalent to positions 24-27 of SEQ ID NO: 9; and
(ii) one or more amino acid mutations in positions equivalent to positions 49-51 of SEQ ID NO: 9,
wherein the mutant BBTI (BBKI) polypeptide:
(a) binds selectively to a ligand that does not bind to the corresponding unmutated polypeptide; and
(b) is resistant to cleavage by pepsin.

In another aspect, the invention provides a mutant AMTI polypeptide (e.g. a mutant of a polypeptide comprising an amino acid sequence as set forth in SEQ ID NO: 10 or a variant thereof, such as a polypeptide having at least 80% sequence identity to SEQ ID NO: 10, e.g. an isoform of AMTI) comprising two or more amino acid mutations compared to the corresponding unmutated polypeptide (i.e. SEQ ID NO: 10 or variant thereof), wherein the mutant AMTI polypeptide comprises:
(i) one or more amino acid mutations in positions equivalent to positions 23-26 of SEQ ID NO: 10; and
(ii) one or more amino acid mutations in positions equivalent to positions 47-49 of SEQ ID NO: 10,
wherein the mutant AMTI polypeptide:
(a) binds selectively to a ligand that does not bind to the corresponding unmutated polypeptide; and
(b) is resistant to cleavage by pepsin.

In another aspect, the invention provides a mutant SSTI polypeptide (e.g. a mutant of a polypeptide comprising an amino acid sequence as set forth in SEQ ID NO: 11 or a variant thereof, such as a polypeptide having at least 80% sequence identity to SEQ ID NO: 11, e.g. an isoform of SSTI) comprising two or more amino acid mutations compared to the corresponding unmutated polypeptide (i.e. SEQ ID NO: 11 or a variant thereof), wherein the mutant SSTI polypeptide comprises:
(i) one or more amino acid mutations in positions equivalent to positions 26-30 of SEQ ID NO: 11; and
(ii) one or more amino acid mutations in positions equivalent to positions 50-52 of SEQ ID NO: 11,
wherein the mutant SSTI polypeptide:
(a) binds selectively to a ligand that does not bind to the corresponding unmutated polypeptide; and
(b) is resistant to cleavage by pepsin.

An equivalent position in the mutant SBTI family polypeptide of the invention is preferably determined by reference to the corresponding unmutated polypeptide. In some embodiments, the equivalent position is determined by reference to the amino acid sequence set forth in one of SEQ ID NOs: 1-13, as applicable. The equivalent position can be readily deduced by lining up the sequence of the mutant polypeptide and the sequence of the corresponding unmutated polypeptide (e.g. one of SEQ ID NOs: 1-13) based on the homology or identity between the sequences, for example using a BLAST algorithm.

A “domain” refers to a discrete, continuous part or subsequence of a polypeptide. Thus, a polypeptide sequence containing more than one domain as defined herein potentially can form an independent, stable folding unit and may be associated with one or more functions. Thus, in the context of the polypeptides of the invention, a domain may contain one of the specified structural components of an SBTI family polypeptide as defined above, e.g. a loop or a beta-strand. Accordingly, a polypeptide of the invention contains multiple domains as defined herein that can form a beta-trefoil structure defined above. For instance, the first and second domains as defined herein with respect to the unmutated polypeptides may be viewed as loops or parts thereof, e.g. amino acid sequences interposed between beta-strands that form loops (or parts thereof) connecting said beta-strands. The first and second domains in the mutated polypeptides may be viewed as ligand binding domains, i.e. domains that contribute to binding of the mutant polypeptide to a target ligand.

Thus, a domain may be viewed as a “region” of the polypeptide of the invention containing one or more polypeptide elements, e.g. ligand binding functionality and/or a connective loop. The terms “domain” and “region” may be used interchangeably herein.

The mutant polypeptide of the present invention comprises two or more mutations compared to a corresponding unmutated (e.g. wild-type) SBTI family polypeptide, e.g. compared to SEQ ID NOs: 1-13, 62 or 85, wherein at least one mutation is in each of the first and second domains defined above. Although other modifications may be made to the mutant polypeptide compared to the unmutated polypeptide in addition to the mutations in the first and second domains, the mutant polypeptide must be resistant to cleavage by pepsin. Thus, the mutant polypeptides of the invention may have other mutations, i.e. insertions, deletions and/or substitutions, as compared to the corresponding unmutated polypeptide in addition to the one or more mutations in the first and second domains.

Whilst not wishing to be bound by theory, it is thought that the structure of the polypeptides described herein contributes to their stability and resistance to cleavage by pepsin. Thus, mutations to the polypeptide outside of the first and second domains should not disrupt the overall structure of the polypeptide, particularly the beta-barrel structure, relative to the corresponding unmutated polypeptide.

As discussed above, the polypeptides of the invention contain 12 beta-strands that contribute to the structure of the polypeptide. In particular, beta-strands 1, 4, 5, 8, 9 and 12 (numbered from the N- to C-terminus) form the beta-barrel. Thus, in some embodiments, the mutant polypeptide does not contain any mutations in beta-strands corresponding to beta-strand numbers 1, 4, 5, 8, 9 and 12 in Table 1. In some embodiments, the mutant polypeptide does not contain mutations in any of the beta-strands. However, some mutations may be tolerated in the beta-strand domains, particularly domains 2, 3, 6, 7, 10 and 11. Thus, in some embodiments, one or more beta-strand domains (e.g. 1-6, 1-4, e.g. 2 or 3 domains), such as one or more of beta-strand domains 2, 3, 6, 7, 10 and 11, may contain one or more mutations, e.g. 1, 2 or 3 mutations. In some preferred embodiments, mutations in the beta-domains are conservative substitutions.

Thus, in some embodiments, the mutant polypeptides of the invention may contain one or more mutations in the domains connecting the beta-strands. In particular, the inventors have determined that some specific loops (so-called “other” domains, i.e. domains other than the first and second domains defined above) may tolerate mutations, including non-conservative substitutions and/or insertions, without affecting the overall structure of the polypeptide and/or its resistance to degradation (cleavage) by pepsin. Thus, any one or more of the following domains (referring to the beta-strand numbering in Table 1) may contain one or more mutations, e.g. 1, 2 or 3 mutations, in the mutant polypeptide of the invention:
(i) the domain N-terminal to (i.e. upstream of) beta-strand 1;
(ii) the domain between beta-strands 2 and 3;
(iii) the domain between beta-strands 4 and 5;
(iv) the domain between beta-strands 5 and 6; and
(v) the domain between beta-strands 8 and 9.

More particularly, the mutant SBTI family polypeptide of the invention may comprise:
(i) one or more amino acid mutations in a domain corresponding to positions 6-9 of SEQ ID NO: 1;
(ii) one or more amino acid mutations in a domain corresponding to positions 36-38 of SEQ ID NO: 1;
(iii) one or more amino acid mutations in a domain corresponding to positions 63-65 of SEQ ID NO: 1;
(iv) one or more amino acid mutations in a domain corresponding to positions 84-87 of SEQ ID NO: 1; and/or
(v) one or more amino acid mutations in a domain corresponding to positions 124-128 of SEQ ID NO: 1.

In some embodiments, positions corresponding to positions 8, 86 and/or 126 of SEQ ID NO: 1 are not mutated or comprise only conservative substitutions in the mutant SBTI family polypeptide of the invention.

The one or more mutations in these “other” domains may provide the mutant polypeptide with additional functionality. As a representative example, mutations in the other domains may improve the ligand binding characteristics of the mutant polypeptide, e.g. improve the association and/or dissociation rate of the polypeptide to the ligand of interest. In some embodiments, mutations in the other domains may form a second ligand binding domain (see e.g. Examples 6-8).

Thus, in some embodiments, the mutant SBTI family polypeptide of the invention may comprise:
(i) one or more amino acid mutations in a domain corresponding to positions 6-9 of SEQ ID NO: 1; and/or
(ii) one or more amino acid mutations in a domain corresponding to positions 124-128 of SEQ ID NO: 1.

As discussed above, SBTI family polypeptides typically contain at least one disulfide bridge, e.g. 1-3 disulfide bridges, which contribute to the structure of the polypeptides. Thus, where the corresponding unmutated polypeptide contains one or more disulfide bridges, it is preferred that the cysteine residues that form the disulfide bridges are conserved in the mutant polypeptide. As a representative example, SBTI (e.g. SEQ ID NO: 1) contains two disulfide bridges formed between cysteine residues at positions 39 and 86, and positions 136 and 145 of SEQ ID NO: 1. Thus, in some embodiments, the mutant SBTI family polypeptide (e.g. a mutant polypeptide derived from SBTI, e.g. SEQ ID NO: 1) contains cysteine residues at positions corresponding to positions 39, 86, 136 and 145 of SEQ ID NO: 1.

Notably, the BBTI/BBKI polypeptide does not contain any disulfide bridges. Thus, the cysteine residue in this polypeptide may be mutated (e.g. substituted) without disrupting disulfide bridges (see e.g. SEQ ID NO: 85). Additionally or alternatively, a cysteine residue may be introduced to the BBTI/BBKI polypeptide without disrupting disulfide bridges. The introduction of a cysteine residue, e.g. outside of the ligand binding domains of the mutant polypeptide, may improve the functionality of the polypeptide, e.g. facilitate the conjugation of labels (e.g. fluorescent probes) and/or drugs to the mutant polypeptide.

As shown in the examples, the inventors have determined that the first and second domains as defined herein (and other domains, such as the domain corresponding to positions 124-128 of SEQ ID NO: 1) may be modified significantly to provide ligand binding activity and, unexpectedly, this does not affect the advantageous properties associated with the SBTI family polypeptides, e.g. resistance to degradation (i.e. cleavage) by pepsin. In this respect, based on the data in the Examples below, it is expected that the domains tolerate non-conservative substitutions at all positions and the insertion of additional residues at any position.

Accordingly, each non-beta strand domain defined herein, particularly the first and second domains as defined herein, may independently comprise two or more amino acid mutations, particularly substitutions (i.e. conservative or non-conservative) and/or insertions.

As discussed above, the size of the domains differs between SBTI family polypeptides. Thus, the domains may independently contain three or more, e.g. 3, 4, 5 or 6 substitutions. For instance, in some embodiments, all of the amino acids in the first and/or second domain are substituted.

Additionally or alternatively, the domains may independently contain one or more, e.g. 1-15, 1-12, 1-10 or 1-8, amino acid insertions, such as 1-6, 1-4 or 1-3 amino acid insertions.

While it is contemplated that one or more amino acids in the domains may be deleted, it is preferred that the mutations to the domains are substitutions and/or insertions, particularly in the first and second domains of the mutant SBTI family polypeptide.

As all of the amino acids in the first and second domains may be substituted and additional amino acids also may be inserted, the mutant SBTI family polypeptide of the invention may be alternatively viewed as comprising replacement amino acid sequences in the domains defined herein. In other the words, the first and/or second domains (and optionally other domains defined herein, such as the domain corresponding to positions 124-128 of SEQ ID NO: 1) may be replaced with amino acid sequences of at least the same length as the domain in the unmutated (wild-type) SBTI polypeptide, such as with a sequence of consisting 2-25 amino acids, e.g. 2-20, 3-20, 4-20, e.g. 4, 5, 6, 7, 8, 9, 10, 11, 12, 13, 14 or 15 amino acids. In some embodiments, the replacement sequence does not contain amino acids that correspond to the amino acids at the equivalent positions in the unmutated SBTI family polypeptide.

Accordingly, the invention may be viewed as providing a mutant Kunitz-type soybean trypsin inhibitor (SBTI) family polypeptide comprising:
(i) an amino acid sequence in a first domain corresponding to positions 22-25 of SEQ ID NO: 1 that is different to the amino acid sequence in the corresponding unmutated SBTI family polypeptide; and
(ii) an amino acid sequence in a second domain corresponding to positions 47-50 of SEQ ID NO: 1 that is different to the amino acid sequence in the corresponding unmutated SBTI family polypeptide,
wherein the mutant SBTI family polypeptide:
(a) binds selectively to a ligand that does not bind to the corresponding unmutated (e.g. wild-type) SBTI family polypeptide; and
(b) is resistant to cleavage by pepsin.

As discussed above, the amino acid sequences in the first and second domains may consist of 2-25 amino acids, e.g. 2-20, 3-20, 4-20, e.g. 4, 5, 6, 7, 8, 9, 10, 11, 12, 13, 14 or 15 amino acids. As a representative example, where the mutant SBTI family polypeptide is a mutant SBTI polypeptide (e.g. a mutant of a polypeptide comprising the amino acid sequence set forth in SEQ ID NO: 1), the amino acid sequence in the first domain is not DITA (SEQ ID NO: 26) and the amino acid sequence in the second domain is not RNEL (SEQ ID NO: 27). It will be evident that equivalent statements for other SBTI family polypeptides disclosed herein may be derived from the amino acid sequences set forth in SEQ ID NOs: 2-11 and the positions of the domains in these sequence in Table 2 above.

In some embodiments, any mutations that are present in the mutant SBTI family polypeptide of the present invention outside of the first and second domains, and optionally outside the other non-beta-strand domains discussed above, are conservative amino acid substitutions. A conservative amino acid substitution refers to the replacement of an amino acid by another which preserves the physicochemical character of the polypeptide (e.g. D may be replaced by E or vice versa, N by Q, or L or I by V or vice versa). Thus, a substituting amino acid outside of the first and second domains may have similar properties to the amino acid that is replaced (e.g. similar hydrophobicity, hydrophilicity, electronegativity, bulky side chains etc.). The substituting amino acid may have similar properties particularly in domains of the mutant polypeptide that contribute to the structure (e.g. and the resistance to degradation by pepsin) of the polypeptide (e.g. the beta-strands, especially beta-strands 1, 4, 5, 8, 9 and 12). Isomers of the native L-amino acid e.g. D-amino acids may be incorporated.

It will be appreciated that it is not essential to retain the protease inhibitory activity of the unmutated SBTI family polypeptide in the mutant SBTI family polypeptide of the invention. As the mutant SBTI family polypeptides of the invention may find particular utility as orally-administered therapeutics, diagnostics or nutraceuticals, it may be advantageous to eliminate or reduce the protease inhibitory activity, i.e. to prevent side-effects associated with inhibiting digestive enzymes in the subject. Thus, in some embodiments, the mutant SBTI family polypeptide may comprise a mutation that eliminates or substantially reduces the protease inhibitory activity of the polypeptide relative to the corresponding unmutated SBTI family polypeptide. As discussed above, the protease inhibitory activity may be serine protease inhibitory activity, particularly trypsin and/or chymotrypsin inhibitory activity.

As a representative example, the key amino acid in SBTI (SEQ ID NO: 1) for its trypsin inhibitory activity is the arginine residue at the position corresponding to position 63 of SEQ ID NO: 1. Thus, this residue may be mutated, e.g. substituted or deleted, in the mutant polypeptide of the invention to eliminate or substantially reduce the trypsin inhibitory activity. Accordingly, in some embodiments, the mutant SBTI family polypeptide (e.g. derived from a polypeptide comprising an amino acid sequence set forth in SEQ ID NO: 1 or a variant thereof, e.g. SEQ ID NO: 12 or 13) comprises a substitution or deletion at a position equivalent to position 63 of SEQ ID NO: 1. In some embodiments, the substitution is a non-conservative substitution. In some embodiments, the arginine residue is substituted with a residue selected from alanine, glutamine and glutamate.

A mutation that eliminates or substantially reduces the protease inhibitory activity of the mutant SBTI family polypeptide refers to a mutation that reduces the protease inhibitory activity by at least 60%, e.g. at least 70%, 80%, 90%, 95% or 99%, relative to the activity of the corresponding unmutated SBTI family polypeptide, when tested under the same conditions, e.g. substrate, temperature, pH, concentration etc. Protease inhibitor assays are well-known in the art and any suitable assay may be selected by the skilled person to determine the effect of a mutation on the protease inhibitory activity, depending on the SBTI family polypeptide being tested.

Thus, in some embodiments, the mutant SBTI family polypeptide of the invention comprises an amino acid sequence with at least 70% (e.g. 75%, 80%, 85%, 90% or 95%) sequence identity to an amino acid sequence of any one of SEQ ID NOs: 1-13, wherein the polypeptide contains one or more mutations in the first and second domains as defined herein.

Alternatively viewed, the mutant SBTI family polypeptide of the present invention may differ from a corresponding unmutated SBTI family polypeptide (e.g. a polypeptide comprising an amino acid sequence as set forth in any one of SEQ ID NOs: 1-13) by, for example, 2 to 65, 2 to 60, 2 to 55, 2 to 50, 2 to 45, 2 to 40, 2 to 35, 2 to 30, 2 to 25, 2 to 20, 2 to 15, 2 to 10, or 2 to 8 amino acid substitutions, insertions and/or deletions, preferably substitutions and/or insertions. For instance, the mutant SBTI family polypeptide of the present invention may comprise 1 to 30, 1 to 25, 1 to 20, 1 to 15, 1 to 10, 1 to 8, 1 to 6, 1 to 5, 1 to 4, e.g. 1, 2 to 3 amino acid substitutions and/or 1 to 30, 1 to 25, 1 to 20, 1 to 15, 1 to 10, 1 to 8, 1 to 6, 1 to 5, 1 to 4, e.g. 1, 2 to 3 amino acid insertions, wherein the first and second domains each comprise at least 1 mutation, i.e. at least one substitution and/or insertion.

In some embodiments, it is preferred that any deletions are at the N- and/or C-terminus, i.e. truncations, thereby generating portions of SBTI family polypeptides, e.g. SEQ ID NOs: 1-13.

As noted above, in some embodiments, the mutant SBTI family polypeptide of the invention that satisfies the sequence identity and/or number of mutation parameters set forth above does not contain any mutations in beta-strands 1, 4, 5, 8, 9 and 11 (using the numbering in Table 1 above). In some embodiments, the mutant SBTI family polypeptide of the invention does not contain mutations in any of the beta-strands. Moreover, it is preferred that cysteine residues involved in disulfide bonds in the corresponding unmutated SBTI family polypeptide are conserved in the mutant polypeptide.

While any substitutions and insertions are contemplated, particularly in the first and second domains as defined herein, it will be understood that the introduction of cysteine residues (by substitution or insertion) can result in undesirable side reactions, which may affect the functionality of the polypeptide. Accordingly, in some embodiments, the substitution and/or insertion does not introduce a cysteine residue into the mutant SBTI family polypeptide (i.e. relative to the corresponding unmutated SBTI family polypeptide). However, as noted above, the introduction of a cysteine residue into a mutant BBKI polypeptide of the invention may be desirable.

As shown in the Examples, the inventors have shown that an SBTI family polypeptide can be provided with new ligand binding functionality by mutating the first and second domains of the polypeptide as defined herein. In this respect, the mutant SBTI family polypeptide of the invention is able to bind selectively to a ligand (i.e. target molecule of interest) that is not bound by the corresponding unmutated SBTI family polypeptide. As discussed below, targets of particular interest include molecules that are found in the gastrointestinal (GI) tract of an animal (e.g. a bird, fish or mammal, e.g. a human), as the pepsin-resistant properties of the mutant polypeptides of the invention allow the polypeptides to transit through the GI tract and retain their ligand binding function.

While SBTI family polypeptides bind to molecules in the GI tract, e.g. digestive proteases, such as serine proteases (e.g. trypsin and/or chymotrypsin), the mutant SBTI family polypeptides of the invention bind selectively to other molecules. Thus, while the mutant SBTI family polypeptides of the invention may retain their ability to bind to digestive proteases, such as serine proteases (e.g. trypsin and/or chymotrypsin), these enzymes typically do not represent the target ligand for the ligand binding activity conferred by the first and second domains of the mutant polypeptide of the invention. Thus, in some embodiments, the mutant polypeptide of the invention may viewed as binding selectively to more than one ligand, e.g. two ligands, i.e. a digestive protease, such as a serine protease (e.g. trypsin and/or chymotrypsin), and a further ligand (i.e. the target of interest). As discussed below, while the further ligand (i.e. target of interest) may be a digestive enzyme, it will be a different enzyme to the enzyme that is bound by the corresponding unmutated SBTI family polypeptide.

Thus, in some embodiments, the mutant SBTI family polypeptide of the invention binds selectively to a ligand that is not bound by unmutated (e.g. wild-type) SBTI family polypeptides, particularly the corresponding unmutated (e.g. wild-type) SBTI family polypeptides. In particular, the mutant SBTI family polypeptide of the invention may be defined as binding selectively to a ligand that is not bound by a polypeptide comprising an amino acid sequence as set forth in any one of SEQ ID NOs: 1-13, 62 or 85 under comparable conditions.

The term “binds selectively” refers to the ability of the mutant polypeptide to bind non-covalently (e.g. by van der Waals forces and/or hydrogen-bonding) to its ligand (i.e. target of interest) with greater affinity and/or specificity than to other components in the environment (i.e. surroundings or setting) in which mutant polypeptide and ligand are present (e.g. in the GI tract or a sample (e.g. biological, e.g. clinical or experimental, sample) comprising the ligand). Thus, the mutant polypeptide of the invention may alternatively be viewed as binding specifically and reversibly to its target ligand, e.g. a GI tract ligand, under suitable conditions.

Binding of the mutant polypeptide to the target ligand may be distinguished from binding to other molecules present in its environment. The mutant polypeptide of the invention either does not bind to other molecules present in its environment or does so negligibly or non-detectably, so that any such non-specific binding, if it occurs, may be readily distinguished from binding to the target ligand.

In particular, if the mutant polypeptide of the invention binds to molecules other than the target ligand (with the exception of the natural ligand of corresponding unmutated SBTI family polypeptide, to which the mutant polypeptide may still bind), such binding must be transient and the binding affinity must be less than the binding affinity of the mutant polypeptide for the target ligand. Thus, the binding affinity of the mutant polypeptide for the target ligand should be at least an order of magnitude more than the other molecules present in its environment (with the exception of the natural ligand of corresponding unmutated SBTI family polypeptide, to which the mutant polypeptide may still bind). Preferably, the binding affinity of the mutant polypeptide for the target ligand should be at least 2, 3, 4, 5, or 6 orders of magnitude more than the binding affinity for other molecules present in the environment (with the exception of the natural ligand of corresponding unmutated SBTI family polypeptide, to which the mutant polypeptide may still bind).

Alternatively viewed, the binding affinity of mutant polypeptide of the invention for the target ligand is at least an order of magnitude more than the corresponding unmutated SBTI family polypeptide (e.g. a polypeptide comprising or consisting of an amino acid sequence as set forth in any one or SEQ ID NOs: 1-13) under the same conditions. Accordingly, the corresponding unmutated SBTI family polypeptide does not bind, e.g. bind selectively, to the ligand (i.e. the target of interest) or vice versa.

Accordingly, if the corresponding unmutated SBTI family polypeptide binds to the target ligand, such binding must be transient and the binding affinity must be less than the binding affinity of the mutant polypeptide for the target ligand and optionally less than the binding affinity of the unmutated SBTI family polypeptide for its native ligand, e.g. protease, such as trypsin or chymotrypsin. Thus, the binding affinity of corresponding unmutated SBTI family polypeptide for the target ligand should be at least 2, 3, 4, 5, or 6 orders of magnitude less than the binding affinity of the mutant polypeptide for the target ligand and optionally less than the binding affinity of the unmutated SBTI family polypeptide for its native ligand.

Thus, selective (or specific) binding refers to affinity of the mutant SBTI family polypeptide of the invention for the target ligand where the dissociation constant (K_{d}) of the mutant polypeptide for the target ligand is less than about 10⁻³ M. In a preferred embodiment the dissociation constant of the mutant polypeptide for the target ligand is less than about 10⁻⁵ M or 10⁻⁶ M, e.g. less than about 10⁻⁷ M or 10⁻⁸ M. The K_{d} may be determined using any suitable means known in the art. For instance, the K_{d} may be determined using surface plasmon resonance (SPR), e.g. using a Biacore T200 or equivalent, on a solution of mutant polypeptide in a suitable buffer (e.g. phosphate buffered saline (PBS)) at 25° C., e.g. as described in the Examples.

Suitably therefore, the dissociation constant of a corresponding unmutated SBTI family polypeptide (e.g. a polypeptide comprising or consisting of an amino acid sequence as set forth in any one or SEQ ID NOs: 1-13, 62 or 85) for the target ligand typically is more than about 10⁻⁵ M. In a preferred embodiment the dissociation constant of the unmutated SBTI family polypeptide for the target ligand is more than about 10⁻⁴ M or 10⁻³ M. The K_{d} may be determined using any suitable means known in the art, e.g. as described above.

The term “resistant to cleavage by pepsin” means that the mutant SBTI family polypeptide of the invention is not substantially cleaved when contacted with pepsin under suitable conditions, i.e. conditions suitable for the pepsin enzyme to function. In some embodiments, less than about 30%, e.g. less than about 25%, 20%, 15% or 10% of the mutant SBTI family polypeptide of the invention is cleaved by the pepsin enzyme, preferably less than about 5% is cleaved.

Suitable conditions include conditions that result in the cleavage of a control polypeptide, e.g. a polypeptide susceptible to cleavage by pepsin. In some embodiments, the control polypeptide may be an antibody or a fragment or derivative thereof. For instance, suitable conditions include conditions that result in the cleavage of at least about 80%, e.g. at least about 85%, 90%, 95% or 99%, of the control polypeptide.

In a representative embodiment, suitable conditions include incubating the polypeptide(s) in a solution comprising about 1.0 mg/ml (e.g. a final concentration of about 3,000 U/mL) of pepsin (e.g. pepsin from pig gastric mucosa or pepsin from chicken) at about 20-45° C., such as about 30-40° C., e.g. about 25° C. or about 37° C. (e.g. for pig or human pepsin) or about 40° C. (e.g. for chicken pepsin), preferably about 37° C. for about 5-15 minutes, e.g. about 10 minutes. The pepsin solution may contain any buffer suitable for pepsin activity, e.g. 50 mM glycine-HCl, pH 2.2. The concentration of the test polypeptides may be any suitable range to determine the level of cleavage, such as an amount visible on an SDS-PAGE gel with Coomassie staining, e.g. about 1-50 μM, such as about 2-20 μM, or via a Western blot, e.g. about 150-450 nM, such as about 200-300 nM.

Cleavage of the mutant SBTI family polypeptide of the invention and/or control may be measured using any suitable means known in the art. For instance, as described in the Examples, the cleavage reaction may be stopped by denaturing the pepsin enzyme, e.g. by heat, and the amount of polypeptide cleaved may be measured, e.g. by SDS-PAGE and image analysis, ELISA etc. Conveniently, the amount of polypeptide cleaved may be compared to the amount of the polypeptide incubated under the same conditions without the pepsin enzyme. The mutant SBTI family polypeptide of the invention may be viewed as resistant to cleavage by pepsin when less than about 30%, e.g. less than about 25%, 20%, 15% or 10% of the mutant SBTI family polypeptide of the invention is cleaved by the pepsin, e.g. less than about 5% is cleaved under the conditions set out above.

It will be evident from the Examples that incubating the mutant SBTI family polypeptide of the invention with pepsin for a longer period of time, e.g. 30, 45, 60, 75, or 100 minutes may result in a higher amount of cleavage. However, in comparison to other polypeptides, which may be degraded to undetectable levels under the conditions set out above or even under much lower concentrations of pepsin e.g. 1 mg/ml pepsin, the polypeptides of the invention show remarkable resistance to pepsin. In this respect, even when the majority of the polypeptide of the invention is degraded in the conditions set out above, this may still enable an effective amount of polypeptide to be delivered to its site of action in the GI tract. Thus, in some embodiments, the mutant SBTI family polypeptide of the invention may be viewed as resistant to cleavage by pepsin when about 80% or less, e.g. about 75%, 70%, 65%, 60%, 55%, 45% 45%, 40%, 35% or less of the mutant SBTI family polypeptide of the invention is cleaved by the pepsin, e.g. under the conditions set out above.

Pepsin is typically isolated from the gut mucosa of pigs (Sus scrofa). Thus, in preferred embodiments, the mutant SBTI family polypeptide of the invention is resistant to cleavage by pepsin isolated from the gut mucosa of pigs. However, it is expected that the mutant SBTI family polypeptide of the invention will be resistant to cleavage by other pepsin enzymes, e.g. human or chicken pepsin.

Thus, in some embodiments, the mutant SBTI family polypeptide of the invention is resistant to cleavage by any enzyme or combination of enzymes in the enzyme commission number 3.4.23.1.

Representative pepsin enzymes include the following, which list refers to the UniProtKB/Swiss-Prot accession numbers: P03954, PEPA1_MACFU; P28712, PEPA1_RABIT; P27677, PEPA2_MACFU; P27821, PEPA2_RABIT; PODJD8, PEPA3_HUMAN; P27822, PEPA3_RABIT; PODJD7, PEPA4_HUMAN; P27678, PEPA4_MACFU; P28713, PEPA4_RABIT; PODJD9, PEPA5_HUMAN; Q9D106, PEPA5_MOUSE; P27823, PEPAF_RABIT; P00792, PEPA_BOVIN; Q9N2D4, PEPA_CALJA; Q9GMY6, PEPA_CANLF; P00793, PEPA_CHICK; P11489, PEPA_MACMU; P00791, PEPA_PIG; Q9GMY7, PEPA_RHIFE; Q9GMY8, PEPA_SORUN; P81497, PEPA_SUNMU; and P13636, PEPA_URSTH.

In some embodiments, the mutant SBTI family polypeptide of the invention is resistant to cleavage by at least one pepsin enzyme selected from the following group, which refers to the UniProtKB/Swiss-Prot accession numbers: PODJD8, PEPA3_HUMAN; PODJD7, PEPA4_HUMAN; PODJD9, PEPA5_HUMAN; and P00791, PEPA_PIG.

As shown in the Examples below, the inventors have determined that the SBTI family polypeptides have other advantageous properties that may make the mutant SBTI family polypeptides of the invention particularly useful for enteral, particularly oral, administration. In particular, it is expected that the mutant SBTI family polypeptides of the invention show resistance to cleavage by other digestive proteases (particularly pancreatic proteases, e.g. trypsin, chymotrypsin and elastase), stability in bile acids, thermal resilience and high stability in low pH environments.

Thus, in some embodiments, the mutant SBTI family polypeptide of the invention is resistant to cleavage by protease enzymes in pancreatin (e.g. pig pancreatin), e.g. trypsin, chymotrypsin and/or elastase, e.g. less than about 30%, e.g. less than about 25%, 20%, 15% or 10% of the mutant SBTI family polypeptide of the invention is cleaved by pancreatin under suitable conditions.

Suitable conditions include conditions that result in the cleavage of a control polypeptide, e.g. a polypeptide susceptible to cleavage by pancreatin (e.g. pig pancreatin). In some embodiments, the control polypeptide may be an antibody or a fragment or derivative thereof. For instance, suitable conditions include conditions that result in the cleavage of at least about 80%, e.g. at least about 85%, 90%, 95% or 99%, of the control polypeptide.

In a representative embodiment, suitable conditions include incubating the polypeptide(s) in a solution comprising about 0.1-10 mg/ml pancreatin (e.g. pig pancreatin) at about 30-40° C., e.g. about 37° C., for about 20-40 minutes, e.g. about 30 minutes. The pancreatin solution may contain any buffer suitable for protease enzymes in pancreatin, e.g. 50 mM Tris-HCl, pH 6.8 containing 10 mM CaCl₂). The concentration of the test polypeptides may be any suitable range to determine the level of cleavage, such as an amount visible on an SDS-PAGE gel with Coomassie staining, e.g. about 1-50 μM, such as about 2-20 μM or 5-10 μM, or via a Western blot, e.g. about 150-450 nM, such as about 200-300 nM.

Thus, in some embodiments, the mutant SBTI family polypeptide of the invention is resistant to cleavage by elastase, e.g. less than about 30%, e.g. less than about 25%, 20%, 15% or 10% of the mutant SBTI family polypeptide of the invention is cleaved by elastase under suitable conditions.

Suitable conditions include conditions that result in the cleavage of a control polypeptide, e.g. a polypeptide susceptible to cleavage by elastase. In some embodiments, the control polypeptide may be an antibody or a fragment or derivative thereof. For instance, suitable conditions include conditions that result in the cleavage of at least about 80%, e.g. at least about 85%, 90%, 95% or 99%, of the control polypeptide.

In a representative embodiment, suitable conditions include incubating the polypeptide(s) in a solution comprising about 10 U/mL of elastase (e.g. elastase from pig pancreas) at about 30-40° C., e.g. about 37° C., for about 20-40 minutes, e.g. about 30 minutes. The elastase solution may contain any buffer suitable for elastase activity, e.g. 50 mM Tris-HCl, pH 6.8 containing 10 mM CaCl₂). The concentration of the test polypeptides may be any suitable range to determine the level of cleavage, such as an amount visible on an SDS-PAGE gel with Coomassie staining, e.g. about 1-50 μM, such as about 2-20 μM or 5-10 μM.

In some embodiments, the mutant SBTI family polypeptide of the invention is able to bind selectively to its target ligand following contact with physiological concentrations (e.g. up to about 10 mM) of one or more bile acids, e.g. one or more of sodium glycocholate hydrate, sodium glycodeoxycholate, sodium taurocholate hydrate or sodium taurodeoxycholate hydrate. Contact with one or more bile salts may involve incubation of the mutant SBTI family polypeptide of the invention in a buffered solution (e.g. 50 mM Tris-HCl pH 8.0) containing one or more bile salts for about 20 min at about 25° C.

In some embodiments, the mutant SBTI family polypeptide of the invention is able to bind selectively to its target ligand following contact with a low pH environment, e.g. a pH of about 3.0 or less, such as about 2.5 or less or about 2.0. Contact with a low pH environment may involve incubation of the mutant SBTI family polypeptide of the invention in a buffered solution (e.g. 50 mM Tris-HCl) at a pH of about 3.0 or less for about 20 min at about 20-40° C., such as about 25° C. or 37° C.

In some embodiments, the mutant SBTI family polypeptide of the invention is able to bind selectively to its target ligand following contact with a high temperature environment, e.g. a temperature of about 75° C. or more, such as about 75-100° C. Contact with a high temperature environment may involve incubation of the mutant SBTI family polypeptide of the invention in a buffered solution (e.g. 50 mM Tris-HCl pH 8.0 with 100 mM NaCl) for about 10 min at a high temperature, e.g. about 75-100° C.

As discussed above, the mutant SBTI family polypeptide of the invention has properties that make it particularly useful for oral administration. However, the skilled person would understand that the stability of the polypeptide across a wide variety of conditions may facilitate its use in other environments. For instance, it is thought that highly stable polypeptides may be less immunogenic. Thus, the mutant SBTI family polypeptide of the invention may also find utility as a medicament administered via parenteral routes, e.g. injection or infusion. Accordingly, the mutant SBTI family polypeptide of the invention may find utility in binding ligands in any environment or sample.

Accordingly, the terms “ligand”, “target ligand” and “target of interest” are used interchangeably herein to refer to any substance (e.g. molecule) or entity that it is desired to bind using the mutant SBTI family polypeptide of the invention. Thus, the ligand may be any biomolecule or chemical compound that it may be desired to bind, for example a peptide or protein, polysaccharide, nucleic acid molecule or a small molecule, particularly organic small molecules. The ligand may be a cell or a microorganism, including a virus, or a fragment or product thereof, e.g. a molecule linked to the surface of a cell or microorganism or a toxin produced by a microorganism. It will be seen therefore that the ligand can be any substance or entity for which a specific binding partner (e.g. an affinity binding partner) can be developed. It is sufficient that the ligand is capable of binding at least one binding partner. As shown in the Examples, the mutant SBTI family polypeptide of the invention may find particular utility in the binding of peptides or polypeptides.

Thus, ligands of particular interest may thus include proteinaceous molecules such as peptides, polypeptides, proteins or prions or any molecule which includes a protein or polypeptide component, etc., or fragments thereof. The ligand may be a single molecule or a complex that contains two or more molecular subunits, which may or may not be covalently bound to one another, and which may be the same or different. Thus, in addition to cells or microorganisms, such a complex ligand may also be a protein complex or protein interaction. Such a complex or interaction may thus be a homo- or hetero-multimer. Aggregates of molecules, e.g. proteins, may also be target ligands, for example aggregates of the same protein or different proteins. The ligand may also be a complex between proteins or peptides and nucleic acid molecules, such as DNA or RNA.

A target ligand may be found in any biological or clinical samples, e.g. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared or they may be prior-treated in any convenient way e.g. for storage.

In preferred embodiments, the target ligand is an in vivo ligand, i.e. a ligand found in an organism, particularly an animal, e.g. a mammalian animal such as a human, a bird or a fish.

As the mutant SBTI family polypeptide of the invention is able to withstand the harsh conditions of the gastrointestinal (GI) tract, it will find particular utility in binding to ligands found in the GI. Thus, in a particularly preferred embodiment, the target ligand is a gastrointestinal (GI) tract ligand.

A GI tract ligand refers to any suitable ligand that is found in the GI tract of an animal. A GI tract ligand may be any molecule or entity produced by the animal (e.g. a polypeptide, such as a cytokine, chemokine or a receptor thereof, a digestive enzyme etc.) or a molecule or entity that has been introduced to the GI tract (e.g. a microorganism, such as a bacterium, a virus or protozoa).

The ability to bind selectively to ligands in the GI facilitates the use of the mutant SBTI family polypeptide of the invention in numerous fields, including diagnosis, therapy and nutrition.

For instance, a mutant SBTI family polypeptide of the invention that binds to a biomarker associated with a disease of the GI tract may find utility in the diagnosis of such as disease. In a representative example, the mutant SBTI family polypeptide of the invention that binds selectively to a ligand that is associated with a disease or condition of the GI tract may be conjugated to an imaging agent. The mutant polypeptide: imaging agent conjugate may be administered (e.g. orally) to a subject suspected of having a disease or condition of the GI tract, wherein it will bind to the biomarker, if present. Detection of the mutant polypeptide: imaging molecule, e.g. using imaging techniques, would enable the detection of the presence or absence of the biomarker and allow the subsequent diagnosis of the subject as having the disease or condition of the GI tract, or not.

Similarly, a mutant SBTI family polypeptide of the invention that binds selectively to a biomarker associated with a particular organ of the GI tract (e.g. mouth, oesophagus, stomach, small intestine, large intestine, or anus) or a portion or part thereof may find utility in the imaging of that organ or a portion or part thereof, e.g. using a mutant SBTI family polypeptide of the invention that binds selectively to a biomarker associated with a particular organ of the GI tract or a portion or part thereof conjugated to an imaging agent.

Imaging techniques to detect features of interest (e.g. tumours) in vivo often use imaging agents which are administered to the patient and which serve to improve or enhance the resultant images so that any features of interest (e.g. tumours) are more readily detectable. Imaging methods such as X-ray, computerised tomography (CT) scans, positron emission tomography (PET) and magnetic resonance imaging (MRI) have for many years employed such imaging agents. Although images that provide useful information can be obtained in the absence of imaging agents, the use of these agents can vastly improve the images that are obtained and hence improve the information that is obtained from carrying out the imaging. For example on an MRI image taken without a contrast agent, tumours from about 1-2 centimetres in size and larger can easily be detected. However, it is desirable to be able to detect smaller tumours and hence contrast-enhanced imaging is advantageous.

In order to be able to function as imaging agents, the relevant agents must enter or interact with the tissue of the feature of interest (e.g. tumour) itself. In such cases the success of the detection method will rely on the ability of the imaging agent to access or make contact with the tissue of the feature of interest (e.g. tumour). The administration of a mutant polypeptide of the invention conjugated to an imaging agent to a patient will allow imaging agents to be targeted to a particular feature of interest (e.g. a tumour) thereby improving the imaging of the feature relative to known imaging methods.

In a further embodiment therefore, the present invention provides a method of detecting or imaging a feature of interest (e.g. a tumour) in a patient (e.g. in the GI tract of a patient) comprising administering (e.g. orally) to said patient a mutant SBTI family polypeptide of the invention that binds to a biomarker associated with the feature of interest (e.g. a biomarker associated with a disease or condition of the GI tract) conjugated to a signal generating (e.g. imaging) agent.

Alternatively stated, the invention provides a mutant SBTI family polypeptide of the invention that binds to a biomarker associated with the feature of interest (e.g. a biomarker associated with a disease or condition of the GI tract) conjugated to a signal generating (e.g. imaging) agent for use in imaging a feature of interest in a patient (e.g. for detecting the presence or absence of tumours in a patient). The mutant SBTI family polypeptide of the invention that binds to a biomarker associated with the feature of interest conjugated to a signal generating agent may be formulated for oral administration to said patient.

In all cases, a further step of recording the signal (e.g. obtaining an image) of the patient may be carried out to detect or image the feature of interest (e.g. detect the presence or absence of said tumour). This step of recording a signal also forms a further optional step in the above method and use.

The image thus recorded or obtained can be analysed in order to examine the feature of interest, e.g. to determine whether it is indicative of the presence of a tumour and thereby it can be determined whether a tumour is present or absent.

By “detecting the presence or absence of a tumour”, it is meant carrying out steps to determine whether or not a tumour can be observed, e.g. using imaging techniques such as MRI. The image of the patient that is obtained by carrying out the imaging technique is thus observed and it is determined whether the resultant image is indicative of the presence of a tumour, or whether it is indicative of the absence of a tumour (or the absence of a tumour that is of such a size as can be detected by that particular technique). It is expected that very small tumours (e.g. less than 0.1 mm in diameter) will not be detectable and as such a conclusion that there is no tumour is in fact a conclusion that no tumour of a detectable size is present.

This can be performed by reference to appropriate controls and/or references, such as patients who are known not to have a tumour, or to other regions of the patient's body which do not have a tumour.

Administration of the mutant SBTI family polypeptide of the invention that binds to a biomarker associated with the feature of interest (e.g. a biomarker associated with a disease or condition of the GI tract) conjugated to a signal generating (e.g. imaging) agent allows the signal generating agent to be concentrated at the feature of interest (e.g. tumour), thereby enabling the feature of interest (e.g. tumour) to be imaged (or for the image generated to be improved relative to an image generated without the use of the mutant polypeptide: signal generating agent conjugate).

This can be done for example by X-ray, CT scan or MRI.

Thus, in some embodiments, the ligand is associated with a disease or condition of the GI tract, such as a biomarker associated with said disease or condition. In some embodiments, the ligand is a biomarker associated with an inflammatory disease or condition of the GI tract or a neoplastic disease or condition of the GI tract.

Thus, in a further aspect, the invention provides a mutant SBTI family polypeptide of the invention for use in diagnosis.

Alternatively viewed, the invention provides a method of diagnosing a disease of condition in a subject, the method comprising administering a mutant SBTI family polypeptide of the invention (or a pharmaceutical composition of the invention as defined herein) to a subject in need thereof.

Advantageously, the mutant SBTI family polypeptide of the invention may be conjugated to a signal generating (e.g. imaging) agent.

In some embodiments, the mutant SBTI family polypeptide of the invention finds utility in the diagnosis of an inflammatory disease or condition of the GI tract or a neoplastic disease or condition of the GI tract. An inflammatory disease or condition of the GI tract may include Inflammatory Bowel Disease (IBD, including Crohn's disease and ulcerative colitis) and coeliac disease. A neoplastic disease or condition of the GI tract may include oesophageal, gastric and colorectal cancer.

A “signal generating agent” as referred to herein is an agent, wherein by virtue of its association with the feature of interest (e.g. tissue or tumour) provides a detectable signal, or enhances an existing signal (e.g. radiation, light) and the increased signal (relative to normal) can be used to image the feature of interest, e.g. to establish the presence or absence of a tumour. Preferably the signal generating agent is an imaging agent.

The “imaging agent” is any agent which is used to obtain or to produce or to enhance an image of a patient, such as an agent which is used to obtain or to produce or to enhance an image of a tumour in a patient.

The imaging agent may be an agent which enters or interacts with feature of interest (e.g. organ or tumour tissue) via the interaction between the mutant SBTI family polypeptide and its target ligand on the feature of interest. Thus, mutant SBTI family polypeptide conjugated to a signal generating (e.g. imaging) agent may be viewed as a targeted contrast agent.

Contrast agents are well known and are widely used in imaging techniques to increase the signal difference between the area of interest and background and include gadolinium-based compounds and iron oxide contrast agents (Superparamagnetic Iron Oxide (SPIO) and Ultrasmall Superparamagnetic Iron Oxide (USPIO)).

Examples of appropriate imaging agents include X-ray contrast agents such as Acetrizoic Acid Derivatives, Diatrizoic Acid Derivatives, Iothalamic Acid Derivatives, Ioxithalamic Acid Derivatives, Metrizoic Acid Derivatives, Iodamide, Lypophylic Agents, Aliphatic Acid Salts, Iodipamide, Ioglycamic Acid, Ioxaglic Acid Derivatives, Metrizamide, Iopamidol, lohexol, Iopromide, Iobitridol, Iomeprol, Iopentol, Ioversol, Ioxilan, Iodixanol, Iotrolan, MRI contrast agents such as gadopentetate dimeglumine, gadoteridol, gadoterate meglumine, mangafodipir trisodium, gadodiamide, Gadopentetic acid, Gadoteric acid, Gadolinium, Mangafodipir, Gadoversetamide, Ferric ammonium citrate, Gadobenic acid, Gadobutrol, Gadoxetic acid, Superparamagnetic, Ferumoxsil, Ferristene, Iron oxide, nanoparticles, Perflubron, Ultrasound agents such as Microspheres of human albumin, Microparticles of galactose, Perflenapent, Microspheres of phospholipids and Sulfur hexafluoride. Positron Emission Tomography (PET) and Single photon emission computed tomography (SPECT) agents that are normally excluded from the brain may also be used.

Preferably the imaging is carried out in a non-invasive manner (e.g. by MRI, X-ray, SPECT, PET or CT scan).

In a further aspect, the mutant SBTI family polypeptide of the invention may find utility in therapy.

For instance, the mutant SBTI family polypeptide of the invention may have a direct therapeutic effect by binding to a target ligand, e.g. it may function as an agonist or antagonist of the target ligand, e.g. a ligand associated with a disease or condition, e.g. a disease or condition of the GI tract. Alternatively, the mutant SBTI family polypeptide of the invention may have an indirect therapeutic effect, e.g. by targeting a therapeutic molecule conjugated to the mutant polypeptide of the invention to a disease site by binding to a target ligand at the disease site.

For instance, a mutant SBTI family polypeptide of the invention that binds to a target ligand associated with a disease of the GI tract may find utility in the treatment of such a disease. In a representative example, the mutant SBTI family polypeptide of the invention may bind selectively to a ligand that is associated with a disease or condition of the GI tract, such as a signalling molecule (e.g. a cytokine or chemokine or a receptor thereof), thereby inhibiting the signal provided by said molecule. Thus, the mutant SBTI family polypeptide of the invention may be viewed as a neutralising agent, e.g. to prevent the activity or function of a signalling molecule.

Alternatively, the mutant SBTI family polypeptide of the invention may bind (e.g. directly) to a host-cell surface protein, e.g. a receptor, and function to activate the receptor. Thus, the mutant SBTI family polypeptide of the invention may be viewed as a receptor agonist, e.g. to increase the activity or function of a signalling molecule. For instance, activation of the receptor may induce a specific response that has a therapeutic effect, e.g. the release of a hormone by enteroendocrine cells to affect metabolism or appetite.

Thus, in some embodiments, the target ligand is a signalling molecule, e.g. a receptor or its cognate ligand. For instance, the target ligand may be a cytokine, chemokine or a receptor thereof. In particular, the signalling molecule may be associated with an inflammatory disease or condition or neoplastic disease or condition.

In another representative example, the mutant SBTI family polypeptide of the invention that binds to a target ligand associated with a disease of the GI tract (e.g. biomarker) may by conjugated to a therapeutic agent, i.e. an agent with a therapeutic utility, e.g. a cytotoxic agent or a radioisotope. The mutant polypeptide: therapeutic agent conjugate may be administered (e.g. orally) to a subject having a disease or condition of the GI tract, e.g. a neoplastic disease or condition, wherein it will bind to the target ligand, thereby bringing the therapeutic agent into proximity with the tissue expressing the target ligand. In other words, the mutant SBTI family polypeptide of the invention may be used to deliver a therapeutic agent to a disease site associated with, e.g. expressing, the target ligand (e.g. biomarker).

The inflammatory disease or condition may be an inflammatory disease or condition of the GI tract. An inflammatory disease or condition of the GI tract may include Inflammatory Bowel Disease (IBD, including Crohn's disease and ulcerative colitis) and coeliac disease.

The neoplastic disease or condition may be a neoplastic disease or condition of the GI tract. A neoplastic disease or condition of the GI tract may include oesophageal, gastric and colorectal cancer.

The skilled person would appreciate that the mutant SBTI family polypeptide of the invention advantageously may bind selectively to ligands that are not produced by the subject. In particular, the target ligand may be a molecule associated with a microorganism in the gut. In some embodiments, the target ligand may be a molecule associated with a pathogen, such as a bacterium, virus or protozoa. Thus, alternatively viewed, in some embodiments, the mutant SBTI family polypeptide of the invention may find utility in treating or preventing a disease or condition of the GI tract that is caused by a pathogen.

In a representative example, the mutant SBTI family polypeptide of the invention may bind selectively to a toxin produced by a pathogen, e.g. to neutralise the toxin. A toxin produced by a pathogen may be a polypeptide or peptide toxin, such as a polypeptide toxin produced by a bacterium such as Helicobacter pylori, Vibrio cholerae, Escherichia coli, Shigella sp., Salmonella sp., Campylobacter sp. or Clostridium difficile, or a protozoa such as Giardia sp., Entamoeba sp., or Eimeria sp. In some embodiments, the mutant SBTI family polypeptide of the invention binds selectively to a toxin produced by a Clostridium difficile, such as TcdA or TcdB (e.g. to a portion thereof, such as the combined repetitive oligopeptide (CROP) domain of TcdA or the glucosyltransferase domain (GTD) of TcdB).

Other target ligands include small molecule toxins in the diet or released from the host microbiome, such as mycotoxins and aflatoxins.

As shown in the Examples, the inventors have developed mutant SBTI family polypeptides that bind to toxins TcdA and TcdB of Clostridium difficile. These toxins facilitate pathogenesis by disrupting the intestinal epithelium. Accordingly, in a particular aspect, the invention provides:
a polypeptide having at least 80% (e.g. at least 85% or 90%) sequence identity to SEQ ID NO: 1, wherein the mutant polypeptide comprises:
(i) an amino acid sequence as set forth in SEQ ID NO: 28, 30, 32 or 36, preferably SEQ ID NO: 28 or 30 at positions equivalent to positions 22-25 of SEQ ID NO: 1; and
(ii) an amino acid sequence as set forth in SEQ ID NO: 29, 31, 33 or 37, preferably SEQ ID NO: 29 or 31 at positions equivalent to positions 47-50 of SEQ ID NO: 1,
wherein the mutant polypeptide:
(a) binds selectively to TcdA of Clostridium difficile (particularly residues 2304-2710 of the combined repetitive oligopeptide (CROP) domain of C. difficile toxin A); and
(b) is resistant to cleavage by pepsin.

Suitably, the amino acid sequences in (i) and (ii) above are SEQ ID NOs: 28 and 29, SEQ ID NOs: 30 and 31, SEQ ID NOs: 32 and 33 or SEQ ID NOs: 36 and 37, preferably SEQ ID NOs: 28 and 29 or SEQ ID NOs: 30 and 31.

In a further particular embodiment, the invention provides:
a polypeptide having at least 80% (e.g. at least 85% or 90%) sequence identity to SEQ ID NO: 1, wherein the mutant polypeptide comprises:
(i) an amino acid sequence as set forth in SEQ ID NO: 42 at positions equivalent to positions 22-25 of SEQ ID NO: 1 (i.e. the amino acid sequence as set forth in SEQ ID NO: 42 replaces the amino acids at positions equivalent to positions 22-25 of SEQ ID NO: 1); and
(ii) an amino acid sequence as set forth in SEQ ID NO: 43 at positions equivalent to positions 47-50 of SEQ ID NO: 1 (i.e. the amino acid sequence as set forth in SEQ ID NO: 43 replaces the amino acids at positions equivalent to positions 47-50 of SEQ ID NO: 1),
wherein the mutant polypeptide:
(a) binds selectively to TcdB of Clostridium difficile (particularly the glucosyltransferase domain of TcdB); and
(b) is resistant to cleavage by pepsin.

In a further particular embodiment, the invention provides:
a polypeptide having at least 80% (e.g. at least 85% or 90%) sequence identity to SEQ ID NO: 1, wherein the mutant polypeptide comprises:
(i) an amino acid sequence as set forth in SEQ ID NO: 42 at positions equivalent to positions 22-25 of SEQ ID NO: 1 (i.e. the amino acid sequence as set forth in SEQ ID NO: 42 replaces the amino acids at positions equivalent to positions 22-25 of SEQ ID NO: 1);
(ii) an amino acid sequence as set forth in SEQ ID NO: 43 at positions equivalent to positions 47-50 of SEQ ID NO: 1 (i.e. the amino acid sequence as set forth in SEQ ID NO: 43 replaces the amino acids at positions equivalent to positions 47-50 of SEQ ID NO: 1); and
(iii) an amino acid sequence as set forth in SEQ ID NO: 70 or 76 at positions equivalent to positions 124-128 of SEQ ID NO: 1 (i.e. the amino acid sequence as set forth in SEQ ID NO: 70 or 76 replaces the amino acids at positions equivalent to positions 124-128 of SEQ ID NO: 1),
wherein the mutant polypeptide:
(a) binds selectively to TcdB of Clostridium difficile (particularly the glucosyltransferase domain of TcdB); and
(b) is resistant to cleavage by pepsin. Thus, in a further embodiment, the invention provides a polypeptide comprising an amino acid sequence as set forth in any one of SEQ ID NOs: 44-47 or a polypeptide comprising an amino acid sequence with at least 80% (e.g. at least 85%, 90% or 95%) sequence identity to an amino acid sequence as set forth in any one of SEQ ID NOs: 44-47, wherein the first and second domains consist of the sequences as defined above, e.g. SEQ ID NOs: 28, 30, 32 or 36 and SEQ ID NOs: 29, 31, 33 or 37, respectively, wherein the polypeptide binds selectively to TcdA of Clostridium difficile (particularly residues 2304-2710 of the combined repetitive oligopeptide (CROP) domain of C. difficile toxin A).

Thus, in a further embodiment, the invention provides a polypeptide comprising an amino acid sequence as set forth in SEQ ID NO: 51 or a polypeptide comprising an amino acid sequence with at least 80% (e.g. at least 85%, 90% or 95%) sequence identity to an amino acid sequence as set forth in SEQ ID NO: 51, wherein the first and second domains consist of the sequences as defined above, e.g. SEQ ID NO: 42 and SEQ ID NO: 43, respectively, wherein the polypeptide binds selectively to TcdB of Clostridium difficile (particularly the glucosyltransferase domain of TcdB).

Thus, in yet a further embodiment, the invention provides a polypeptide comprising an amino acid sequence as set forth in SEQ ID NO: 81 or 82 or a polypeptide comprising an amino acid sequence with at least 80% (e.g. at least 85%, 90% or 95%) sequence identity to an amino acid sequence as set forth in SEQ ID NO: 81 or 82, wherein the first and second domains consist of the sequences as defined above, e.g. SEQ ID NO: 42 and SEQ ID NO: 43, respectively, wherein a third domain, corresponding to positions 124-128 of SEQ ID NO: 1, consists of an amino acid sequence as set forth in SEQ ID NO 70 or 76, and wherein the polypeptide binds selectively to TcdB of Clostridium difficile (particularly the glucosyltransferase domain of TcdB). In some embodiments, the polypeptide comprises a fourth domain, corresponding to positions 6-9 of SEQ ID NO: 1, which consists of an amino acid sequence as set forth in SEQ ID NO: 63.

It will be evident that the polypeptides described above may find utility in therapy as described herein, particularly in the treatment or prevention of a Clostridium difficile infection in a subject, e.g. a GI tract infection.

In a further representative embodiment, the mutant SBTI family polypeptide of the invention may bind selectively to a molecule on the surface of a microorganism, e.g. pathogen, in the GI tract. For instance, the mutant SBTI family polypeptide of the invention may be used to direct a therapeutic agent, e.g. antibiotic agent or anti-viral agent, conjugated to the mutant polypeptide to a pathogen, e.g. a site of infection in the GI tract. Alternatively, the mutant SBTI family polypeptide of the invention may be conjugated to a molecule that promotes an immune response, such that binding of the mutant polypeptide to the target ligand on the surface of the pathogen functions to target the pathogen for destruction by the host immune system. In some embodiments, the mutant SBTI family polypeptide of the invention may bind selectively to a molecule on the surface of a pathogen, e.g. a virus, to inhibit or reduce the infection. For instance, the mutant SBTI family polypeptide of the invention may bind selectively to a molecule on the surface of a virus to reduce or inhibit the infectivity of the virus.

Thus, in some embodiments, the target ligand is a virus, such as a viral polypeptide, e.g. a capsid polypeptide or portion thereof. In some embodiments, the virus is that infects the GI tract such as a norovirus or rotavirus. In some embodiments, the mutant SBTI family polypeptide of the invention is for use in treating a viral infection, e.g. reducing the viral infection (e.g. the infectivity of the virus) in the subject to be treated.

Thus, in a further aspect, the invention provides a mutant SBTI family polypeptide of the invention for use in therapy.

Alternatively viewed, the invention provides a method of treating a disease of condition in a subject, the method comprising administering a mutant SBTI family polypeptide of the invention (or a pharmaceutical composition of the invention as defined herein) to a subject in need thereof.

Advantageously, the mutant SBTI family polypeptide of the invention may be conjugated to a therapeutic agent. As noted above, therapeutic agents include any agent with a therapeutic utility, including toxins such as cytotoxic agents and radioisotopes.

In some embodiments, the mutant SBTI family polypeptide of the invention binds to a molecule, e.g. a polypeptide, on surface of bacteria in the GI tract in order to modulate the host microbiome.

The skilled person would appreciate that the mutant SBTI family polypeptide of the invention advantageously may bind selectively to other host polypeptides found in the GI tract, such as host digestive enzymes. Thus, in some embodiments, the target ligand is a digestive enzyme. As noted above, SBTI family polypeptides commonly interact with protease enzymes. Thus, in embodiments where the target ligand is a digestive enzyme, it is preferred that the target ligand is not a protease enzyme, particularly not a serine protease, such a trypsin or chymotrypsin, or pepsin. Thus, in some embodiments, the target ligand is a lipase or glycosidase, such as alpha-amylase.

Thus, a further utility of the mutant SBTI family polypeptides of the invention is in nutrition. As noted above, the mutant SBTI family polypeptide of the invention may bind selectively to a target ligand associated with a specific location within the GI tract, e.g. a specific organ of the GI tract or a portion thereof. In this respect, some molecules found in mucus, e.g. polysaccharides, may be characteristic of specific location (e.g. a feature of interest) in the GI tract. Thus, in some embodiments, the target ligand is a polysaccharide (e.g. a polysaccharide associated with a specific location in the GI tract).

In a representative embodiment, a mutant SBTI family polypeptide of the invention that binds to a target ligand associated with a specific location in the GI tract may be conjugated to an enzyme, e.g. a nutritional enzyme such as phytase, a carbohydrase or a protease. Advantageously, this may allow an enzyme to be anchored at a specific location within the GI tract, which may have numerous utilities. For example, anchoring an enzyme to a specific location in the GI tract may function to slow the enzyme's clearance and/or bring the enzyme into proximity to its substrate(s).

In this respect, phytate is the major phosphorus source in wheat and corn, which is a common component of animal feed; about 75% of all phosphorus in the grains is bound within phytate molecules. Accordingly, some animals, such as poultry (e.g. chickens), pigs and fish, can be fed exogenous phytase (i.e. phytase supplemented feed) in order to break down the phytate in their feed to enhance the release of phosphorus from the phytate needed for the animals' growth and development. However, a significant proportion of the phytase provided in animal feed is simply digested by the animals. Thus, in a representative example, a mutant SBTI family polypeptide of the invention that binds a ligand in a specific location in the GI tract (e.g. the gizzard) could be conjugated to an enzyme, e.g. a nutritional enzyme such as phytase, to improve the retention of the enzyme in the GI tract and/or increase the exposure of the enzyme to its substrate, e.g. phytate.

It will be evident that a mutant SBTI family polypeptide of the invention may be conjugated to any enzyme that may find utility in the GI tract of an animal. For instance, a mutant SBTI family polypeptide: enzyme conjugate may be used to provide an subject with an enzyme in which the subject is deficient, i.e. a subject needing enzyme replacement therapy. In a representative example, a subject that is lactose intolerant may be provided with a mutant SBTI family polypeptide conjugated to lactase. In a further representative embodiment, the mutant SBTI family polypeptide may be conjugated to an enzyme capable of degrading harmful molecules in the GI tract, such as gluten (e.g. in subject with coeliac disease) or ethanal (e.g. to alleviate the detrimental effects of alcohol consumption, such as a hangover).

The enzyme conjugated to the mutant SBTI family polypeptide of the invention may be modified to improve its function, e.g. stability and/or activity, in the GI tract. For instance, the enzyme may be cyclized.

Thus, in a further aspect, the invention provides a composition comprising the mutant SBTI family polypeptide of the invention. The composition may be in the form of a pharmaceutical composition. In some embodiments, the pharmaceutical composition is formulated for oral administration. The composition may be in the form of an animal feed, a nutraceutical, a functional food, a dietary supplement or a medical food. The mutant SBTI family polypeptide of the invention in the composition may be conjugated to another molecule as described herein, e.g. a therapeutic agent, a signal generating agent, an enzyme etc.

Alternatively viewed, the invention provides an animal feed, a nutraceutical, functional food, a dietary supplement or medical food comprising the mutant SBTI family polypeptide of the invention. The mutant SBTI family polypeptide of the invention in the nutraceutical, functional food, dietary supplement or medical food may be conjugated to another molecule as described herein, particularly an enzyme.

The identification of foods that have beneficial effects on health has resulted in new terminology to describe the foods and products derived therefrom. For instance, a “nutraceutical” can be defined as a product derived from a food source that provides a physiological benefit and/or provides protection against chronic disease. A nutraceutical is generally sold in a medicinal form not usually associated with food.

A “functional food” is often similar in appearance to, or may be, a conventional food, which is consumed as part of a usual diet and is demonstrated to have physiological benefits and/or reduce the risk of chronic disease beyond basic nutritional functions. Other terms that are used to describe food products that are beneficial for health are “dietary supplements” and “medical foods”.

“Dietary supplements” generally comprise extracts from food sources in a concentrated form and may include: vitamins, minerals, herbs or other botanicals, amino acids, and substances such as enzymes and metabolites. Dietary supplements can be found in many forms such as tablets, capsules, softgels, gelcaps, liquids and powders.

“Medical foods” generally are intended for the specific dietary management of a disease or condition for which there are distinctive nutritional requirements and are typically designed to meet certain nutritional requirements for people diagnosed with specific illnesses. Thus, medical foods can be ingested through the mouth or frequently are administered through tube feeding.

As noted above, the mutant SBTI family polypeptide of the invention may be used to improve the nutrition of a subject and/or the function of the GI tract. Accordingly, it will be evident that some products described herein may be seen to fall within one or more of the above definitions, depending on the form and use of the product. Thus, to some extent, the terms nutraceutical, functional food, dietary supplement or medical food are interchangeable in the context of the nutritional utilities of the invention.

The composition of the invention, particularly the pharmaceutical composition of the invention, typically contains one or more additional pharmaceutically acceptable ingredients, such as an excipient, e.g. a carrier and/or diluent.

“Pharmaceutically acceptable” refers to ingredients that are compatible with other ingredients used in the methods or uses of the invention as well as being physiologically acceptable to the recipient.

As defined herein “treating” or “treatment” as used herein refers broadly to any effect or step (or intervention) beneficial in the management of a clinical condition or disorder. Treatment therefore may refer to reducing, alleviating, ameliorating, slowing the development of, or eliminating one or more symptoms of the disease or condition which is being treated, relative to the symptoms prior to treatment, or in any way improving the clinical status of the subject. A treatment may include any clinical step or intervention which contributes to, or is a part of, a treatment programme or regimen.

A treatment may include delaying, limiting, reducing or preventing the onset of one or more symptoms of the disease, for example relative to the disease or symptom prior to the treatment. Thus, treatment explicitly includes both absolute prevention of occurrence or development of a symptom of the disease, and any delay in the development of the disease or symptom, or reduction or limitation on the development or progression of the disease or symptom.

The “subject” or “patient” is an animal (i.e. any human or non-human animal), preferably a mammal, most preferably a human. In some embodiments, the subject may be a domesticated or farmed animal, e.g. livestock (such as poultry, pigs, cattle, sheep or goats) or farmed fish (e.g. salmon).

Pharmaceutical compositions comprising the mutant SBTI family polypeptide of the invention as described herein may be administered to the subject using any suitable means and the route of administration will depend on the therapeutic agent and disease to be treated. While compositions comprising the mutant SBTI family polypeptides may find particular utility in the GI tract, it will be evident that the compositions may also find utility in other parts of the body. Accordingly, while oral administration is a preferred route of administration for the compositions of the invention, and said compositions therefore may be suitably formulated for oral administration, other routes of administration are contemplated.

Suitably, the composition may be administered systemically or locally.

“Systemic administration” includes any form of non-local administration in which the composition is administered to the body at a site other than the disease site, directly adjacent to, or in the local vicinity of, the disease site, resulting in the whole body receiving the administered composition. Conveniently, systemic administration may be via enteral delivery (e.g. oral or rectal) or parenteral delivery (e.g. intravenous, intramuscular or subcutaneous).

“Local administration” refers to administration of the composition to the body at the site of the disease, at a site directly adjacent to the site of the disease, or in the local vicinity of the disease site, resulting in only part of the body receiving the administered composition. Local administration may be via parenteral delivery (e.g. intratumoral injection, intra-articular injection). Alternatively, local administration may be via enteral delivery (e.g. orally, intra-rectally), e.g. wherein the composition is for administration to the GI tract or a portion or part thereof.

An excipient may include any excipients known in the art, for example any carrier or diluent or any other ingredient or agent such as buffer, antioxidant, chelator, binder, coating, disintegrant, filler, flavour, colour, glidant, lubricant, preservative, sorbent and/or sweetener etc.

The compositions, e.g. pharmaceutical compositions, described herein may be provided in any form known in the art, for example as a liquid, suspension, solution, dispersion, emulsion or any mixtures thereof.

As noted above, the mutant SBTI family polypeptide of the invention may comprise additional sequences. For instance, the mutant polypeptide may contain one or more peptide tags to facilitate purification of the polypeptide, e.g. prior to use in the methods and uses of the invention discussed herein, or to facilitate conjugation of the mutant polypeptide to another molecule or entity (e.g. a therapeutic agent, signal generating agent, enzyme etc.).

Any suitable purification moiety or tag may be incorporated into the polypeptide and such moieties are well known in the art. For instance, in some embodiments, the polypeptide may comprise a peptide purification tag or moiety, e.g. a His-tag sequence. Such purification moieties or tags may be incorporated at any position within the polypeptide. In some preferred embodiments, the purification moiety is located at or towards (i.e. within 5, 10, 15, 20 amino acids of) the N- or C-terminus of the polypeptide.

As noted above, a peptide tag may provide the mutant polypeptide of the invention with additional functionality, e.g. the ability to be conjugated to another molecule or entity. For instance, the mutant polypeptide of the invention may contain a peptide tag that is a capable of forming an isopeptide bond with a peptide or polypeptide tag conjugated to another molecule or entity. For instance, the mutant polypeptide of the invention may contain a tag (e.g. “SpyTag” or “SnoopTag”) peptide or corresponding “Catcher” peptide, such as described in WO2011/098772, WO2016/193746, WO2018/197854, WO2018/189517 and WO2020/183198 all of which are incorporated herein by reference.

The peptide tag may have more than one function, e.g. it may facilitate the conjugation of the mutant polypeptide of the invention to another molecule or entity and function as a purification tag. In some embodiments, the peptide tag may be cleaved prior to the use of the mutant polypeptide of the invention as described herein. In some embodiments, the peptide tag may be cleaved following administration of the mutant polypeptide of the invention to a subject, e.g. by an endogenous protease.

The mutant SBTI family polypeptide of the invention may be conjugated to another molecule or entity to facilitate its use in the utilities and compositions described herein, e.g. in therapy, diagnosis and nutrition. In some embodiments, the mutant polypeptide of the invention is conjugated to a peptide or polypeptide that provides the mutant polypeptide of the invention with an additional functionality, e.g. an enzyme. Conveniently, the additional peptide or polypeptide and mutant polypeptide of the invention may be encoded by a single nucleic acid molecule that, upon expression, generates a fusion protein. Accordingly, in some embodiments, the mutant SBTI family polypeptide is part of (e.g. forms a domain of) a fusion protein.

Alternatively viewed, the invention provides a fusion protein containing: (i) a mutant SBTI family polypeptide of the invention; and (ii) a peptide (e.g. peptide tag as defined above) and/or polypeptide (e.g. enzyme). The mutant SBTI family polypeptide and peptide and/or polypeptide may be separated by one or more linker or spacer sequences.

The precise nature of the linker or spacer sequence is not critical and it may be of variable length and/or sequence, for example it may have 1-40, more particularly 2-20, 1-15, 1-12, 1-10, 1-8, or 1-6 residues, e.g. 6, 7, 8, 9, 10 or more residues. By way of representative example the spacer sequence, if present, may have 1-15, 1-12, 1-10, 1-8 or 1-6 residues etc. The nature of the residues is not critical and they may for example be any amino acid, e.g. a neutral amino acid, or an aliphatic amino acid, or alternatively they may be hydrophobic, or polar or charged or structure-forming e.g. proline. In some preferred embodiments, the linker is a serine and/or glycine-rich sequence.

Exemplary spacer sequences thus include any single amino acid residue, e.g. S, G, L, V, P, R, H, M, A or E or a di-, tri-tetra-penta- or hexa-peptide composed of one or more of such residues.

As mentioned above, the mutant SBTI family polypeptide of the invention may be conjugated to other molecules or entities. Such molecules or entities may be a nucleic acid molecule, protein (e.g. antibody or antigen-binding fragment thereof), peptide, small-molecule organic compound, fluorophore, metal-ligand complex, polysaccharide, nanoparticle, 2D monolayer (e.g. graphene), nanotube, polymer, cell, virus, virus-like particle or any combination of these.

Thus, alternatively viewed, the invention provides a nucleic acid molecule, protein (e.g. antibody or antigen-binding fragment thereof), peptide, small-molecule organic compound, fluorophore, metal-ligand complex, polysaccharide, nanoparticle, 2D monolayer (e.g. graphene), nanotube, polymer, cell, virus, virus-like particle or any combination thereof or solid support conjugated to a mutant SBTI family polypeptide of the invention.

The cell may be a prokaryotic or eukaryotic cell. In some embodiments, the cell is a prokaryotic cell, e.g. a bacterial cell. In some embodiments, the cell is a eukaryotic cell, such as an animal cell, e.g. a human cell.

In some embodiments, the mutant polypeptide of the invention may be conjugated to a compound or molecule which has a therapeutic or prophylactic effect, e.g. an antibiotic, antiviral, vaccine, antitumour agent, e.g. a radioactive compound or isotope, cytokines, toxins, oligonucleotides and nucleic acids encoding genes or nucleic acid vaccines.

In some embodiments, the mutant polypeptide of the invention may be conjugated to a label, e.g. a radiolabel, a fluorescent label, luminescent label, a chromophore label as well as to substances and enzymes which generate a detectable substrate, e.g. horseradish peroxidase, luciferase or alkaline phosphatase. This detection may be applied in numerous assays where antibodies are conventionally used, including Western blotting/immunoblotting, histochemistry, enzyme-linked immunosorbent assay (ELISA), or flow cytometry (FACS) formats. Labels for magnetic resonance imaging, positron emission tomography probes and boron 10 for neutron capture therapy may also be conjugated to the mutant polypeptide.

While it is preferred that peptides and polypeptides are conjugated to the mutant SBTI family polypeptide of the invention via a peptide bond, i.e. in the form of a fusion protein, such that the fusion protein may be genetically encoded, it will be evident that peptides and polypeptides may be conjugated to the mutant SBTI family polypeptide of the invention via other means.

The terms “conjugating” or “linking” in the context of the present invention with respect to connecting the mutant SBTI family polypeptide of the invention to another molecule or entity refers to joining or conjugating said molecules or entities via a chemical bond, typically a covalent bond. Whilst any manner or means of conjugating the mutant SBTI family polypeptide of the invention to another molecule or entity is contemplated herein, as noted above, this may be conveniently achieved by forming an isopeptide bond between a peptide tag in the mutant polypeptide of the invention and corresponding peptide tag or polypeptide “catcher” incorporated in, or linked to, the molecule or entity, e.g. polypeptide, to be conjugated to the mutant polypeptide of the invention.

Thus, the manner or means of conjugating the mutant SBTI family polypeptide of the invention to another molecule or entity may be selected, according to choice, from any number of conjugation or linking means as are widely known in the art and described in the literature. Thus, the mutant polypeptide of the invention may be directly conjugated to the molecule or entity, for example via a domain or moiety of the mutant polypeptide of the invention (e.g. chemically cross-linked). In some embodiments, the mutant polypeptide of the invention may be conjugated indirectly by means of a linker group, or by an intermediary binding group(s) (e.g. by means of a biotin-streptavidin interaction). Thus, the mutant polypeptide of the invention may be covalently or non-covalently linked to the molecule or entity. In preferred embodiments the mutant polypeptide of the invention is conjugated to another molecule or entity via a covalent bond.

The linkage may be a reversible (e.g. cleavable) or irreversible linkage. Thus, in some embodiments, the linkage may be cleaved enzymatically, chemically, or with light, e.g. the linkage may be a light-sensitive linkage.

Linking groups of interest may vary widely depending on the nature of the molecule or entity to be conjugated to the mutant polypeptide of the invention. The linking group, when present, is in many embodiments biologically inert.

Many linking groups are known to those of skill in the art and find use in the invention. In representative embodiments, the linking group is generally at least about 50 daltons, usually at least about 100 daltons and may be as large as 1000 daltons or larger, for example up to 1000000 daltons if the linking group contains a spacer, but generally will not exceed about 500 daltons and usually will not exceed about 300 daltons. Generally, such linkers will comprise a spacer group terminated at either end with a reactive functionality capable of covalently bonding to the solid support.

Spacer groups of interest may include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N, N-diacetic acid, 1,1′-ethylenebis(5-oxo-3-pyrrolidinecarboxylic acid), 4,4′-ethylenedipiperidine, oligoethylene glycol and polyethylene glycol. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linker groups that may find use in the invention include heterofunctional compounds, such as azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl-4-[N-maleimidomethyl]cyclohexane-1-carboxylate, 3-(2-pyridyldithio) propionic acid N-hydroxysuccinimide ester (SPDP), 4-(N-maleimidomethyl)-cyclohexane-1-carboxylic acid N-hydroxysuccinimide ester (SMCC), and the like. For instance, a spacer may be formed with an azide reacting with an alkyne or formed with a tetrazine reacting with a trans-cyclooctene or a norbornene.

In a further aspect, the invention provides a nucleic acid molecule comprising a nucleotide sequence which encodes a polypeptide of the invention as hereinbefore defined.

In some embodiments, the nucleic acid molecule encoding a polypeptide defined above comprises a nucleotide sequence as set forth in any one of SEQ ID NOs: 52-59, 83 or 84 or a nucleotide sequence with at least 80% sequence identity to a sequence as set forth in any one of SEQ ID NOs: 52-59, 83 or 84.

Preferably, the nucleic acid molecule above is at least 85, 90, 95, 96, 97, 98, 99 or 100% identical to the sequence to which it is compared.

Nucleic acid sequence identity may be determined by, e.g. FASTA Search using GCG packages, with default values and a variable pamfactor, and gap creation penalty set at 12.0 and gap extension penalty set at 4.0 with a window of 6 nucleotides. Preferably said comparison is made over the full length of the sequence, but may be made over a smaller window of comparison, e.g. less than 300, 200, 100 or 50 contiguous nucleotides.

The nucleic acid molecules of the invention may be made up of ribonucleotides and/or deoxyribonucleotides as well as synthetic residues, e.g. synthetic nucleotides, that are capable of participating in Watson-Crick type or analogous base pair interactions. Preferably, the nucleic acid molecule is DNA or RNA.

The nucleic acid molecules described herein may be operatively linked to an expression control sequence, or a recombinant DNA cloning vehicle or vector containing such a recombinant DNA molecule. This allows cellular expression of the mutant polypeptide of the invention as a gene product, the expression of which is directed by the gene(s) introduced into cells of interest. Gene expression is directed from a promoter active in the cells of interest and may be inserted in any form of linear or circular nucleic acid (e.g. DNA) vector for incorporation in the genome or for independent replication or transient transfection/expression. Suitable transformation or transfection techniques are well described in the literature. Alternatively, the naked nucleic acid (e.g. DNA or RNA, which may include one or more synthetic residues, e.g. base analogues) molecule may be introduced directly into the cell for the production of the mutant polypeptide of the invention. Alternatively the nucleic acid may be converted to mRNA by in vitro transcription and the relevant protein may be generated by in vitro translation.

Appropriate expression vectors include appropriate control sequences such as for example translational (e.g. start and stop codons, ribosomal binding sites) and transcriptional control elements (e.g. promoter-operator regions, termination stop sequences) linked in matching reading frame with the nucleic acid molecules of the invention. Appropriate vectors may include plasmids and viruses (including both bacteriophage and eukaryotic viruses). Suitable viral vectors include baculovirus and also adenovirus, adeno-associated virus, herpes and vaccinia/pox viruses. Many other viral vectors are described in the art. Examples of suitable vectors include bacterial and mammalian expression vectors pGEX-KG, pEF-neo and pEF-HA.

Thus viewed from a further aspect, the present invention provides a vector, preferably an expression vector, comprising a nucleic acid molecule as defined herein.

As noted above, the nucleic acid molecule may conveniently be fused with DNA encoding an additional peptide or polypeptide, e.g. His-tag, SpyTag, enzyme etc., to produce a fusion protein on expression.

Other aspects of the invention include methods for preparing recombinant nucleic acid molecules according to the invention, comprising inserting a nucleic acid molecule of the invention encoding the polypeptide of the invention into vector nucleic acid.

Nucleic acid molecules of the invention, preferably contained in a vector, may be introduced into a cell by any appropriate means. Suitable transformation or transfection techniques are well described in the literature. Numerous techniques are known and may be used to introduce such vectors into prokaryotic or eukaryotic cells for expression. Preferred host cells for this purpose include prokaryotic cells, such as E. coli. Other host cells include eukaryotic cells such as insect cell lines, yeast and mammalian cell lines. The invention also extends to transformed or transfected prokaryotic or eukaryotic host cells containing a nucleic acid molecule, particularly a vector as defined herein.

Thus, in another aspect, there is provided a recombinant host cell containing a nucleic acid molecule and/or vector as described above.

By “recombinant” is meant that the nucleic acid molecule and/or vector has been introduced into the host cell. The host cell may or may not naturally contain an endogenous copy of the nucleic acid molecule, but it is recombinant in that an exogenous or further endogenous copy of the nucleic acid molecule and/or vector has been introduced.

A further aspect of the invention provides a method of preparing a mutant polypeptide of the invention, which comprises culturing a host cell containing a nucleic acid molecule (e.g. vector) as defined above, under conditions whereby said nucleic acid molecule encoding said polypeptide is expressed and recovering said polypeptide thus produced. The expressed polypeptide forms a further aspect of the invention.

In some embodiments, the mutant polypeptide of the invention may be generated synthetically, e.g. by ligation of amino acids or smaller synthetically generated peptides, or more conveniently by recombinant expression of a nucleic acid molecule encoding said polypeptide as described hereinbefore.

Nucleic acid molecules of the invention may be generated synthetically by any suitable means known in the art.

Thus, the mutant polypeptide of the invention may be an isolated, purified, recombinant or synthesised polypeptide.

The term “polypeptide” is used herein interchangeably with the term “protein”. The term polypeptide or protein typically includes any amino acid sequence comprising at least 40 consecutive amino acid residues, e.g. at least 50, 60, 70, 80, 90, 100, 150 amino acids, such as 40-1000, 50-900, 60-800, 70-700, 80-600, 90-500, 100-400 amino acids.

Standard amino acid nomenclature is used herein. Thus, the full name of an amino acid residue may be used interchangeably with the one letter code or three letter abbreviations. For instance, lysine may be substituted with K or Lys, isoleucine may be substituted with I or Ile, and so on. Moreover, the terms aspartate and aspartic acid, and glutamate and glutamic acid are used interchangeably herein and may be replaced with Asp or D, or Glu or E, respectively.

Whilst it is envisaged that the mutant polypeptide of the invention may be produced recombinantly, and this is a preferred embodiment of the invention, it will be evident that it may be useful to modify one or more residues in the polypeptide, e.g. to improve the stability of the polypeptide. Thus, in some embodiments, the mutant polypeptide of the invention may comprise unnatural or non-standard amino acids.

In some embodiments, the mutant polypeptide of the invention may comprise one or more, e.g. 1, 2, 3, 4, 5 or more non-conventional amino acids, such as 10, 15, 20 or more non-conventional amino acids, i.e. amino acids which possess a side chain that is not coded for by the standard genetic code, termed herein “non-coded amino acids”. Such amino acids are well known in the art, and may be selected from amino acids which are formed through metabolic processes such as ornithine or taurine, and/or artificially modified amino acids such as 9H-fluoren-9-ylmethoxycarbonyl (Fmoc), (tert)-(B)utyl (o)xy (c)arbonyl (Boc), 2,2,5,7,8-pentamethylchroman-6-sulphonyl (Pmc) protected amino acids, or amino acids having the benzyloxy-carbonyl (Z) group.

Examples of non-standard or structural analogue amino acids which may be used in the mutant polypeptide of the invention are D amino acids, amide isosteres (such as N-methyl amide, retro-inverse amide, thioamide, thioester, phosphonate, ketomethylene, hydroxymethylene, fluorovinyl, (E)-vinyl, methyleneamino, methylenethio or alkane), L-N methylamino acids, D-α methylamino acids, D-N-methylamino acids. Further non-standard amino acids which may be used in the polypeptide of, and for use in, the invention are disclosed in Willis and Chin, Nat Chem. 2018; 10 (8): 831-837, in Table 1 of WO2018/189517 and WO2018/197854, all of which are herein incorporated by reference.

As described in detail in the Examples below, the mutant SBTI family polypeptide of the invention may be obtained by selecting a polypeptide that binds to a target ligand of interest from a plurality of polypeptides each having one or more mutations in the domains defined above. Conveniently, the plurality of mutant polypeptides may be encoded by a library of nucleic acid molecules that have been randomly mutagenized in the sequences encoding the domains defined above. The generation of such nucleic acid molecule libraries and the subsequent screening of polypeptides encoded by such libraries are well-known in the art. Any convenient method for producing a plurality of polypeptides suitable for screening may be used to obtain a mutant SBTI family polypeptide of the invention, e.g. phage display, mRNA display, bacterial display, yeast display or ribosome display. Accordingly, any suitable method for screening a plurality of polypeptides having one or more mutations in the domains defined above may be used to obtain a mutant SBTI family polypeptide of the invention, e.g. phage display, mRNA display, bacterial display, yeast display or ribosome display.

Accordingly, in a further aspect the invention provides the use of a nucleic acid molecule encoding an unmutated SBTI family polypeptide as a starting molecule in a mutation and selection screening process for obtaining a mutant SBTI family polypeptide comprising two or more amino acid mutations compared to the corresponding unmutated (e.g. wild-type) SBTI family polypeptide, wherein the mutant SBTI family polypeptide comprises:
(i) one or more amino acid mutations in a first domain corresponding to positions 22-25 of SEQ ID NO: 1; and
(ii) one or more amino acid mutations in a second domain corresponding to positions 47-50 of SEQ ID NO: 1,
and wherein the mutant SBTI family polypeptide:
(a) binds selectively to a ligand that does not bind to the corresponding unmutated (e.g. wild-type) SBTI family polypeptide; and
(b) is resistant to cleavage by pepsin.

In another aspect, the invention provides a library of nucleic acid molecules encoding a plurality of mutant SBTI family polypeptides each comprising two or more amino acid mutations compared to their corresponding unmutated (e.g. wild-type) SBTI family polypeptides, wherein each mutant SBTI family polypeptide comprises:
(i) one or more amino acid mutations in a first domain corresponding to positions 22-25 of SEQ ID NO: 1; and
(ii) one or more amino acid mutations in a second domain corresponding to positions 47-50 of SEQ ID NO: 1.

As noted above, the library of nucleic acid molecules conveniently encodes a plurality of polypeptides in a form that is suitable to be screened to identify polypeptides that bind selectively a target ligand of interest. Thus, the library of nucleic acid molecules may encode a phage display library, an mRNA display library, a bacterial display library, a yeast display library or a ribosome display library. Any suitable form of library may find utility in the present invention. In a preferred embodiment, the library is a phage display library.

Thus, in yet a further aspect, the invention provides a plurality of mutant SBTI family polypeptides encoded by the library of nucleic acid molecules described above.

For instance, where the library is a phage display library, the plurality of polypeptides are displayed on phage particles.

Thus, alternatively viewed, the invention provides a phage display library comprising a plurality of phage particles, wherein each phage particle displays a mutant SBTI family polypeptide comprising two or more amino acid mutations compared to its corresponding unmutated (e.g. wild-type) SBTI family polypeptide, wherein the mutant SBTI family polypeptide comprises:
(i) one or more amino acid mutations in a first domain corresponding to positions 22-25 of SEQ ID NO: 1; and
(ii) one or more amino acid mutations in a second domain corresponding to positions 47-50 of SEQ ID NO: 1.

Any bacteriophage may be used to produce the phage display library of the invention. Suitably, the bacteriophage is a filamentous phage, such as an M13 phage or an fd filamentous phage. Thus, in some embodiments, the vector of the invention is a bacteriophage vector or a phagemid vector.

As the plurality of polypeptides of the invention may be screened to obtain a mutant SBTI family polypeptide that binds selectively to a GI tract ligand as defined above, it may be useful to conduct some or all of the screening/selection steps under conditions found in the GI tract, e.g. low pH, in the presence of digestive enzymes such as pepsin etc. Thus, in some embodiments, the bacteriophage used to produce the phage display library of the invention may be a mutant bacteriophage that is resistant to degradation in such conditions. A mutant bacteriophage that is resistant to degradation in conditions of the GI tract may be obtained by selecting a bacteriophage that is resistant to the conditions of interest from a plurality of mutant bacteriophage.

In a further aspect, the invention provides the use of the library of nucleic acid molecules defined herein or the plurality of mutant SBTI family polypeptides defined herein in a screening method to identify a mutant SBTI family polypeptide that binds selectively to a ligand that does not bind to the corresponding unmutated (e.g. wild-type) SBTI family polypeptide (i.e. a ligand as defined above).

More particularly, the invention provides a method of identifying a mutant SBTI family polypeptide that binds selectively to a ligand of interest as defined above (e.g. a ligand that does not bind to the corresponding unmutated (e.g. wild-type) SBTI family polypeptide) comprising:
(i) providing a plurality of mutant SBTI family polypeptides as defined above;
(ii) contacting the plurality of mutant SBTI family polypeptides of (i) with the ligand of interest; and
(iii) isolating a mutant SBTI family polypeptide that binds selectively to the ligand of interest thereby identifying a mutant SBTI family polypeptide that binds selectively to the ligand of interest.

As the method may be used to identify a mutant SBTI family polypeptide that binds to any target ligand of interest and may use a plurality of mutant SBTI family polypeptides in any form (e.g. phage display library, mRNA display library, bacterial display library, yeast display library or ribosome display library), steps (ii) and (iii) above may be performed using any suitable conditions that may be readily determined by the skilled person.

In a representative embodiment, the method may comprise the steps of:
(i) contacting a plurality of mutant SBTI family polypeptides as defined above (e.g. displayed on a plurality of phage particles) with the ligand of interest under conditions suitable to enable one or more mutant SBTI family polypeptides to bind to the ligand thereby forming non-covalent complexes between said one or more mutant SBTI family polypeptides and the ligand;
(ii) subjecting the non-covalent complexes of (i) to conditions that disrupt non-selective interactions between the mutant SBTI family polypeptides and the ligand;
(iii) isolating mutant SBTI family polypeptides (e.g. isolating the phage particles displaying said polypeptides) that are non-covalently bound to the ligand after step (ii); and
(iv) identifying the mutant SBTI family polypeptides isolated in step (iii) (e.g. isolating and sequencing the nucleic acid molecules encoding the mutant SBTI family polypeptides from the phage particles).

It will be evident that the steps recited above, particularly steps (i), (ii) and (iii), may be repeated (e.g. 1, 2, 3, 4 or more times) under the same or different conditions (e.g. to achieve different levels of stringency). Moreover, additional steps may be included in the method. For instance, negative selection steps may be included to remove phage particles that bind to other ligands, e.g. BSA. The method may include a step of amplifying the mutant SBTI family polypeptides isolated in step (iii), e.g. amplifying the phage encoding the polypeptides, for subsequent screening rounds.

In a representative example, suitable conditions for the step of contacting a plurality of mutant SBTI family polypeptides with the ligand of interest may include incubating the polypeptides (e.g. phage particles displaying the polypeptides) and ligand in a buffered solution, e.g. PBS (pH 6-8) optionally containing a blocking reagent such as BSA, for at least about 1 hour, e.g. 1-10 or 1-5 hours, at 20-30° C. Advantageously, the target ligand may be immobilised on a solid substrate.

Suitable conditions that disrupt non-selective interactions between the mutant SBTI family polypeptides and the ligand include one or more wash steps, e.g. 1-10 or 1-5 wash steps, using a suitable buffer, e.g. the buffer used in the contacting step. A different buffer may be used in more or more wash steps. The buffer used in the wash step may contain other components to disrupt the non-selective interactions, e.g. a salt and/or a surfactant (e.g. a detergent). In some embodiments, the buffer may include excess target ligand in solution (i.e. non-immobilized target ligand), which may be useful for selecting mutant polypeptides with high affinity for the target ligand. Stringent washing conditions may be used and the nature of the stringent washing conditions will depend on the ligand. The skilled person could select such conditions as a matter of routine and representative conditions are set out in the Examples.

Any suitable volume of buffer may be used in the wash step(s). For example, where the ligand is immobilised on a solid substrate, such as beads (e.g. agarose-based beads), the volume of buffer used in the wash steps may be at least about 2 times the volume of the beads, e.g. at least about 3, 4, 5, 6, 7, 8, 9 or 10 times the volume of the beads.

The step of isolating the mutant SBTI family polypeptides that are non-covalently bound to the ligand after step (ii) may be performed using any suitable means and will depend on the form of the mutant polypeptides used to perform the method, e.g. phage display library, mRNA library etc. Suitably, the step of isolating the mutant SBTI family polypeptides may comprise subjecting the polypeptides to conditions suitable to disrupt the polypeptide: ligand complexes, i.e. to disrupt the non-covalent interaction between the polypeptide and the ligand, and subsequently separating the polypeptides from the ligand.

The step of identifying the mutant SBTI family polypeptides isolated in step (iii) may be performed using any suitable means and will depend on the form of the polypeptides used to perform the method, e.g. phage display library, mRNA library etc. Conveniently, the step will involve sequencing the nucleic acid molecule encoding the polypeptide, which is isolated in step (iii).

As discussed above, mutant SBTI family polypeptides of the invention that bind to a specific location in the GI tract may be particularly useful in therapy and nutrition. It will be appreciated that the plurality of mutant SBTI family polypeptides defined above could be used to identity a mutant SBTI family polypeptide that binds selectively to a region of interest (e.g. a feature of interest or a specific location) of the gastrointestinal tract of an animal. Moreover, the skilled person will appreciate that where the region of interest is associated with a disease or condition of the GI tract, the plurality of mutant SBTI family polypeptides defined above could be used to identify a ligand in the GI tract, e.g. a biomarker associated with a disease or condition of the GI tract.

Thus, in a further aspect the invention provides the use of the plurality of mutant SBTI family polypeptides defined herein to:
(i) identify a mutant SBTI family polypeptide that binds selectively to a region of interest of the gastrointestinal tract of an animal; and/or
(ii) identify a ligand in the gastrointestinal tract.

More particularly, the invention provides a method of identifying a mutant SBTI family polypeptide that binds selectively to a region of interest of the gastrointestinal tract of an animal comprising:
(i) administering a plurality of mutant SBTI family polypeptides as defined herein to the gastrointestinal tract of an animal (e.g. orally);
(ii) isolating a mutant SBTI family polypeptide (e.g. a phage particle displaying a mutant SBTI family polypeptide) that is non-covalently bound to the region of interest of gastrointestinal tract of the animal; and
(iii) identifying the mutant SBTI family polypeptide isolated in step (ii).

In a representative embodiment, the plurality of mutant SBTI family polypeptides are displayed on a phage and step (i) involves administering a plurality phage to the GI tract of an animal. As noted above, the phage may be mutated or adapted for administration to the GI tract, e.g. to improve its stability and/or resistance to degradation by conditions found in the GI tract.

The step of isolating a mutant SBTI family polypeptide (e.g. a phage particle displaying a mutant SBTI family polypeptide) that is non-covalently bound to the region of interest of gastrointestinal tract of the animal may involve obtaining a sample of tissue (e.g. a biopsy) from the region of interest (e.g. a tumour) and isolating a mutant SBTI family polypeptide (e.g. a phage particle displaying a mutant SBTI family polypeptide) from the tissue.

In some embodiments, the method is a method for identifying a ligand in the gastrointestinal tract, e.g. a biomarker associated with a disease or condition of the GI tract as defined herein. Accordingly, the method may further comprise a step of identifying the ligand to which the mutant SBTI family polypeptide binds. This may be achieved by any suitable means. For instance, the mutant SBTI family polypeptide may be used to screen a plurality ligands obtained from the region of interest. In this respect, nucleic acid molecules obtained from the region of interest (e.g. from the sample of tissue) may be used to generate a plurality of polypeptides, e.g. a phage display library, which may be screened to identify the ligand(s) that bind to the mutant SBTI family polypeptide.

As used herein, the term “plurality” means two or more, e.g. at least 2, 3, 4, 5, 6, 7, 8, 9, 10, 20 or 30 or more, such as 50, 100, 150, 200, 250, 500, 1000, 10000 or more depending on the context of the invention. For instance, a plurality of mutant polypeptides or phage displaying said polypeptides used in the screening methods described above, may include 105, 106, 107, 108, 109, 1010 or more polypeptides or phage, e.g. 1011, 1012, 1013 or more.

The invention will now be described in more detail in the following non-limiting Examples with reference to the following drawings:

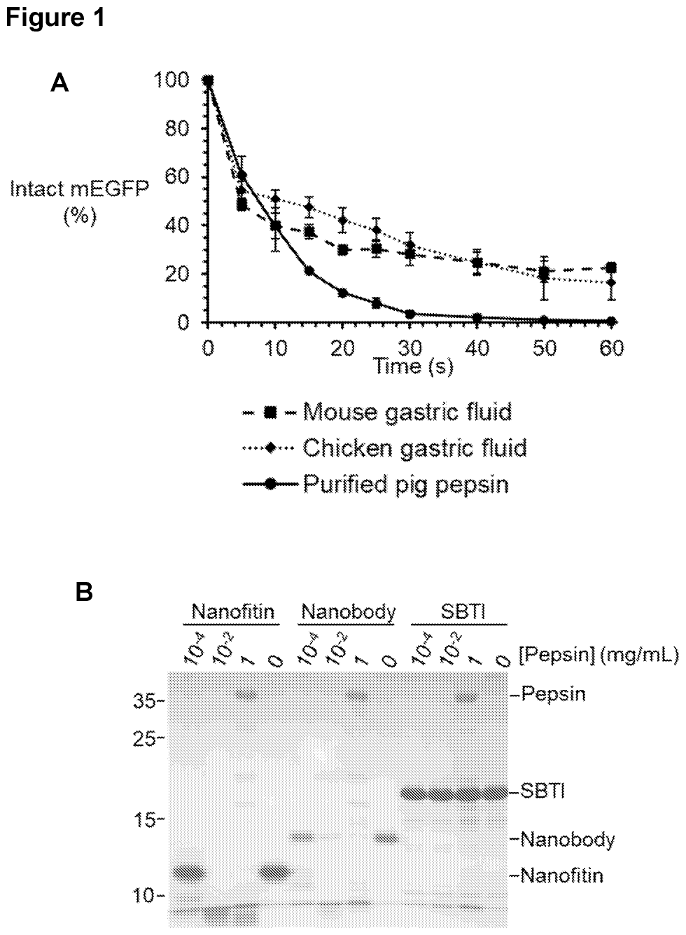

FIG. 1 shows the results of experiments determining protein resistance to gastric concentrations of pepsin. A. Rate of proteolysis in gastric fluid. mEGFP was incubated at 37° C. with 3,028 U/mL pepsin at pH 2.2 or an equal volume of gastric fluid from chicken or mouse. Proteolysis was monitored by fluorescence loss upon digestion of mEGFP, following neutralization of the solution (mean±1 s.d., n=3). B. SBTI was more stable than other scaffolds to pepsin. 20 μM scaffold was incubated with the indicated pepsin concentration for 10 min at 37° C. at pH 2.2, before SDS-PAGE with Coomassie staining.

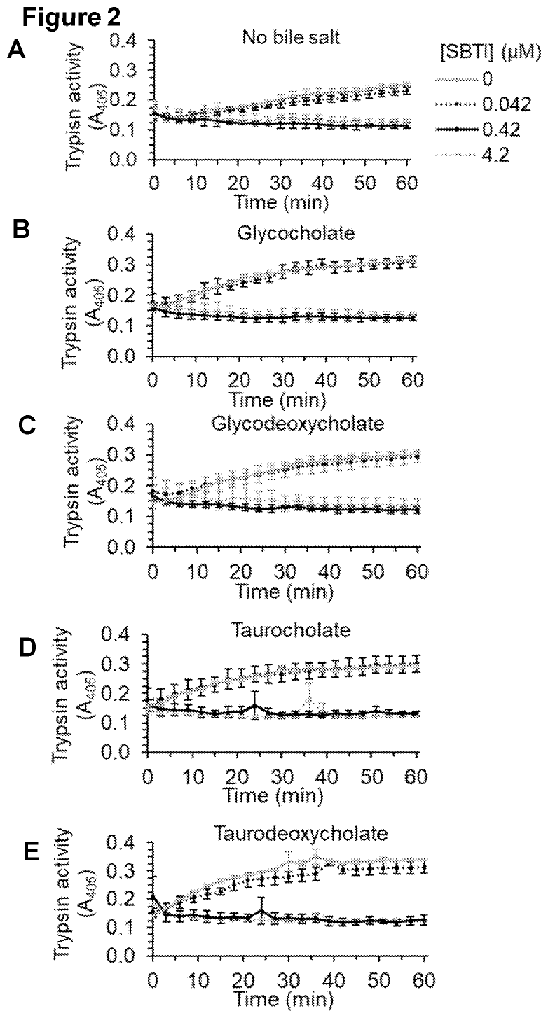

FIG. 2 shows the results of experiments to determine the stability of SBTI to stresses of the GI tract. A-E. SBTI inhibited trypsin even in the presence of bile acids. SBTI at the indicated concentration was incubated with 80 U/mL trypsin±10 mM of different bile acids at pH 8.0 at 37° C. Trypsin activity was monitored by the increase in A₄₀₅ upon cleavage of a chromogenic substrate (mean±1 s.d., n=3).

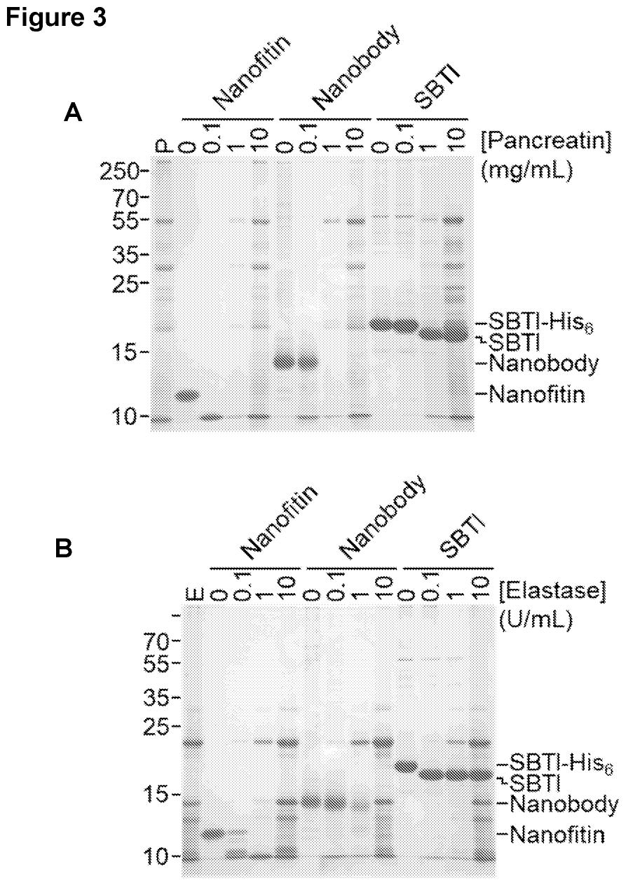

FIG. 3 shows the results of experiments to determine the stability of SBTI to stresses of the GI tract A. SBTI was more resistant than other scaffolds to pancreatin. Pancreatin was incubated at the indicated concentration for 30 min at pH 6.8 and 37° C. with nanofitin, nanobody or SBTI before SDS-PAGE and Coomassie staining. B. SBTI was more resistant than other scaffolds to elastase. Elastase at the indicated concentration was incubated for 30 min at pH 6.8 and 37° C. with nanofitin, nanobody or SBTI before SDS-PAGE and Coomassie staining.

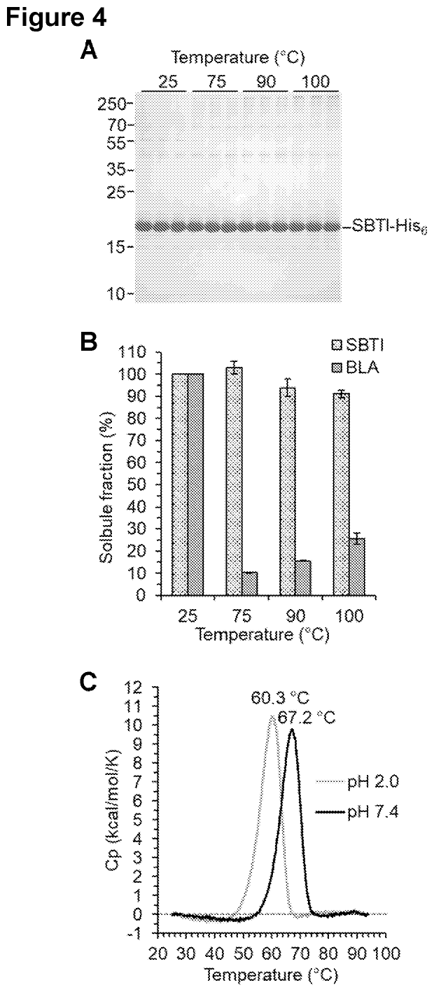

FIG. 4 shows the results of experiments to determine the stability of SBTI to physical stresses. A. SBTI was resilient to boiling. 30 μM SBTI or BLA was incubated at the indicated temperature for 10 min. Aggregated protein was pelleted by centrifugation for 30 min at 16,900 g at 4° C. Soluble protein was analyzed by SDS-PAGE with Coomassie staining. B. Gel densitometry from A (mean±1 s.d., n=3). C. SBTI was thermostable even at pH 2. SBTI unfolding was monitored by DSC in phosphate buffer at pH 2.0 or 7.4. Peak temperature is marked.

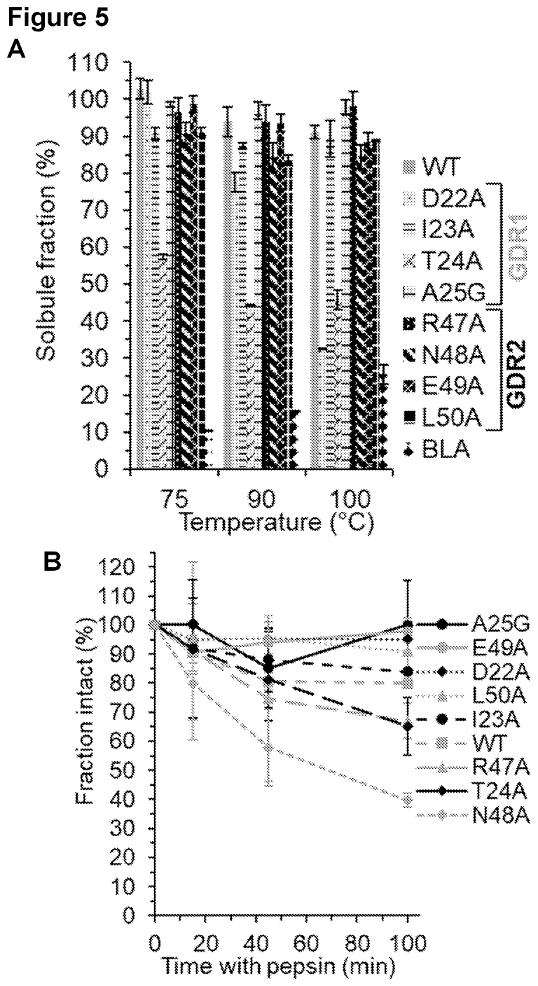

FIG. 5 shows the results of experiments to analysing the effects of mutations in two domains of SBTI. A. Mutants in GDR1 and 2 retained thermal stability. 30 μM SBTI (WT or with the indicated mutation) was incubated at 70, 90 or 100° C. for 10 min. β-lactamase (BLA) was a thermolabile control. Aggregated protein was pelleted by centrifugation and the soluble fraction was analyzed by SDS-PAGE with Coomassie staining. B. Mutants in GDR1 and 2 retained pepsin-resistance. 20 μM SBTI (WT or the indicated mutant) was incubated with 3,028 U/mL pepsin at pH 2.2 at 37° C. for varying times and intact protein was determined by SDS-PAGE with Coomassie staining. Error bars represent mean±1 s.d., n=3.

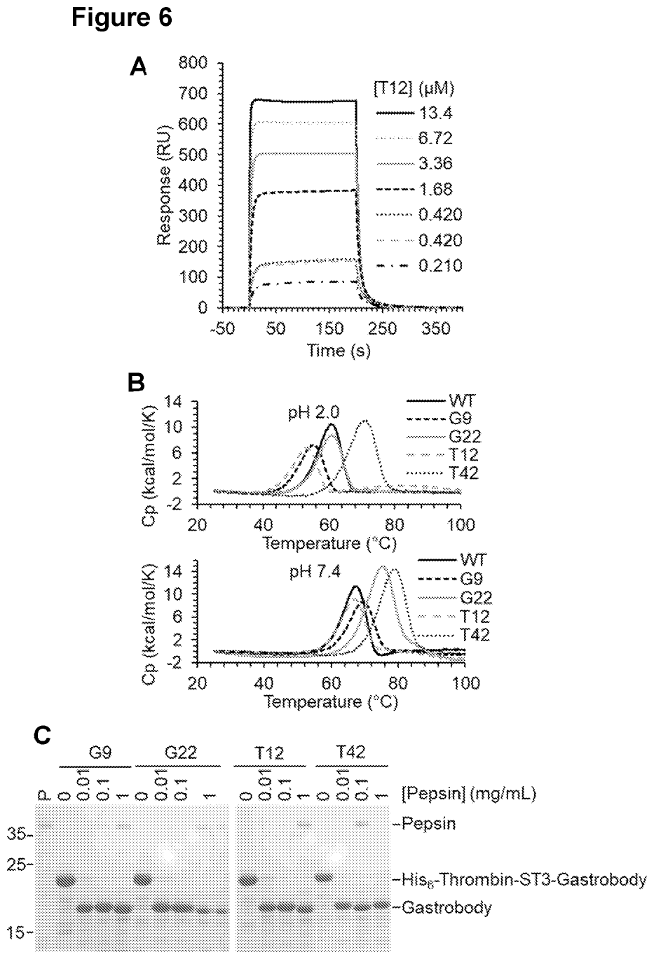

FIG. 6 shows the results of experiments characterising anti-CROP gastrobodies. A. SPR trace from anti-CROP clone T12 binding to immobilized CROP B. Anti-CROP gastrobody thermostability. DSC of four anti-CROP clones compared to WT SBTI at pH 7.4 or 2.0. C. Anti-CROP gastrobodies retained pepsin-resistance. 6 μM anti-CROP gastrobody was incubated with the indicated concentration of pepsin at pH 2.2 and 37° C. for 10 min. Proteins were analyzed by SDS-PAGE with Coomassie staining.

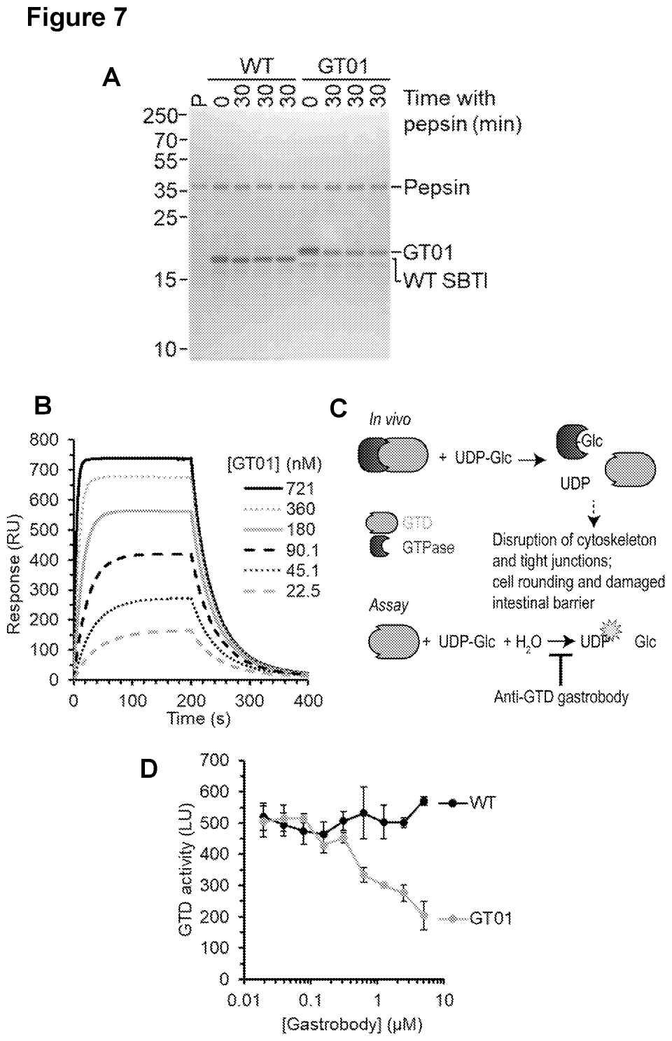

FIG. 7 shows the results of experiments characterizing gastrobodies that bind to the GTD of C. difficile toxin TcdB. A. Pepsin stability of anti-GTD gastrobody. WT SBTI or gastrobody GT01 was incubated with 1 mg/mL pepsin for 30 min in triplicate at 37° C., before SDS-PAGE with Coomassie staining. B. SPR of GT01 binding to immobilized GTD. C. Schematic of GTD activity in vivo and in vitro. TcdB GTD contributes to C. difficile invasion and pathogenesis by monoglucosylating Rho GTPases. In the absence of an acceptor protein, GTD hydrolyzes UDP-glucose. GTD hydrolytic activity can be monitored by luminescent detection of free UDP. D. Inhibition of catalytic activity by anti-GTD gastrobody. Serial dilutions of GT01 or WT SBTI control were incubated with GTD. Hydrolytic activity of GTD was monitored using luminescence (mean±1 s.d., n=3).

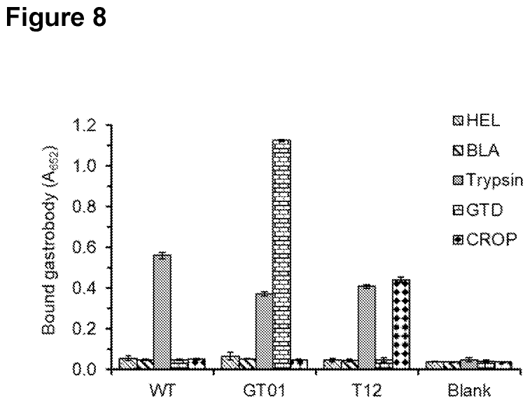

FIG. 8 shows the results of experiments determining whether gastrobodies bind their target specifically. Binding of purified anti-GTD (GT01), anti-CROP (T12) or WT gastrobody to antigen-coated wells was detected by polyclonal anti-SBTI antibody in an ELISA. Antigens were hen egg lysozyme (HEL), β-lactamase (BLA), trypsin, GTD or CROP (mean±1 s.d., n=3).

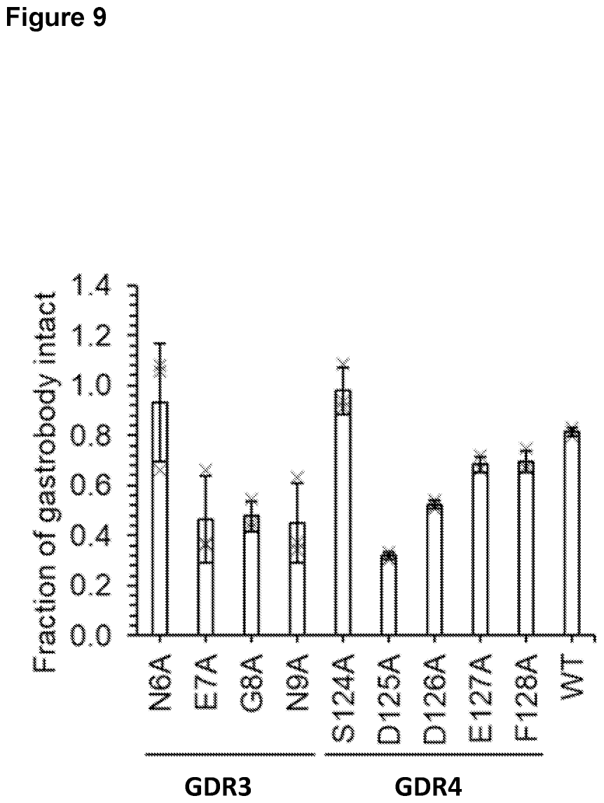

FIG. 9 shows the pepsin-resistance of GDR3/4 alanine mutants. Alanine mutants were incubated with 1 mg/mL pepsin at pH 2.2 and 37° C. for 30 min. Digestion was stopped by boiling in SDS-loading dye. Intact protein was quantified from Coomassie-stained SDS-PAGE with gel densitometry. Samples where digestion was immediately stopped (t=0 min) were set to 1 (mean±1 s.d., n=3 with individual data-points as crosses).

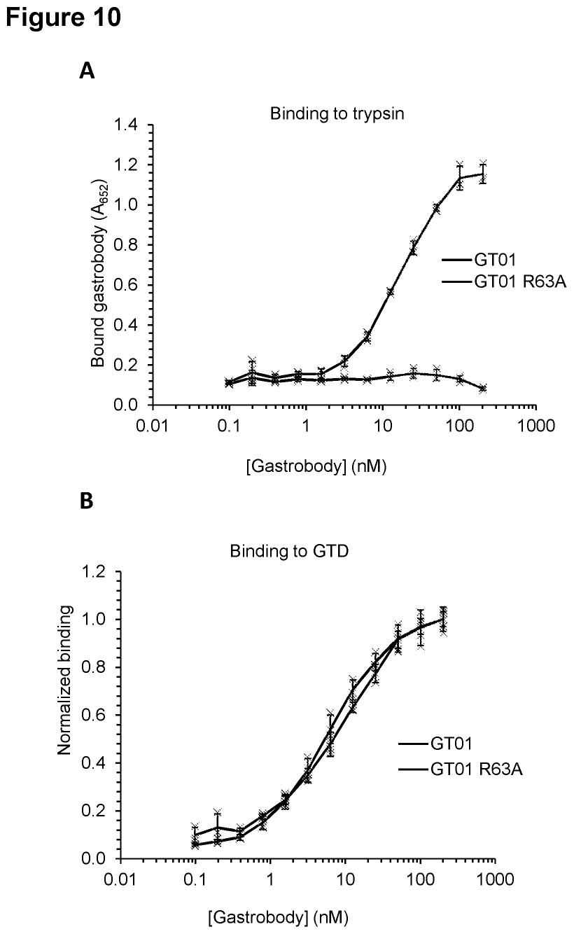

FIG. 10 shows (A) binding of GT01 and GT01 R63A to trypsin-Gastrobodies bound to trypsin-coated wells were detected with biotinylated GTD and streptavidin-HRP in a plate assay (mean±1 s.d., n=3 with individual data-points as crosses); and (B) GT01 and GT01 R63A binding to GTD-Binding of gastrobodies to GTD was analysed by ELISA. Bound gastrobody was detected by anti-SBTI antibody. Absorbance values were normalized to the absorbance at the highest concentration of each gastrobody (mean±1 s.d., n=3 with individual data-points as crosses).

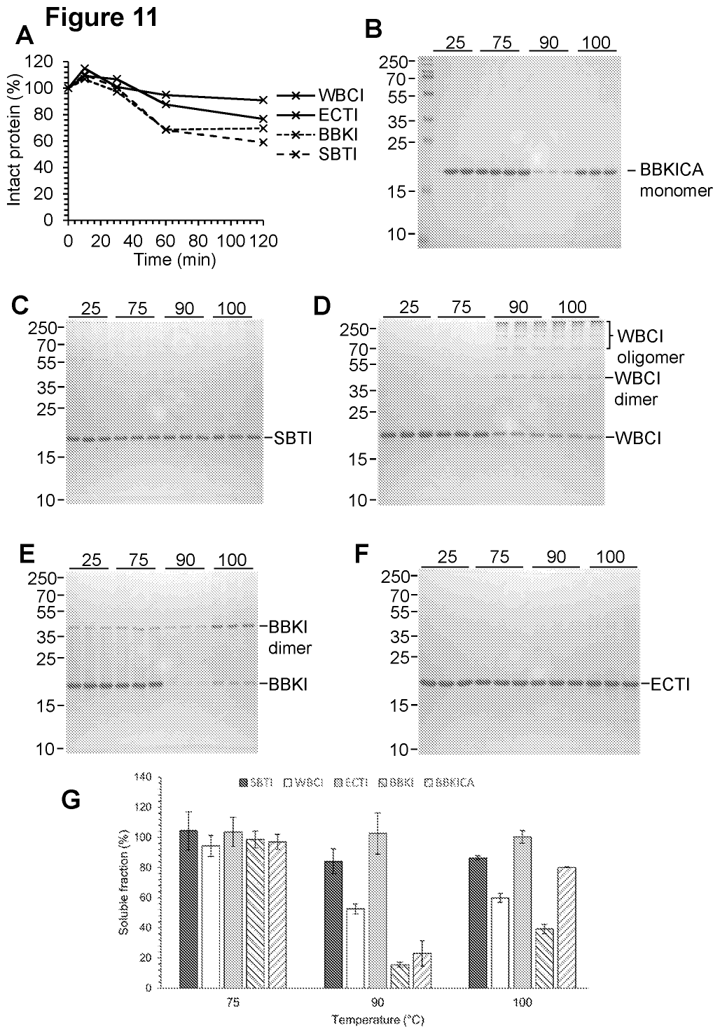

FIG. 11 shows pepsin- and heat-resilience of β-trefoil proteins. (A) Pepsin resistance of structural homologues. SBTI, ECTI, BBKI and WBCI (WCI) were incubated with 1 mg/mL pepsin at pH 2.2 and 37° C. Digestion was stopped by boiling in SDS-loading dye. Digested proteins were run on SDS-PAGE, stained with Coomassie, and intact bands were quantified by gel densitometry. Band intensity at t=0 was set to 100%. N=1. (B-G) Temperature dependent solubility of structural homologues. BBKICA (B), SBTI (C), WBCI (D), BBKI (E) or ECTI (F) was heated to 25, 75, 90 or 100° C. for 10 min. Aggregated protein was pelleted and the soluble fraction was separated on SDS-PAGE (B-F). Band intensity of monomers was quantified by densitometry (G). Intensity of bands of samples at 25° C. was set to 100% (mean±1 s.d., n=3).

### EXAMPLES

### Example 1—Determination of the Rate of Proteolysis in Gastric Fluid

The rate of proteolysis in gastric fluid was analysed to establish a benchmark that could be used to assess protein scaffolds. Pepsin is the principal protease in the human stomach at 0.5-1 mg/mL, where 1 mg contains ≥2,500 U pepsin activity. One unit of pepsin activity is defined to “produce ΔA280 of 0.001 per min at pH 2.0 at 37° C., measured as trichloroacetic acid-soluble products using haemoglobin substrate”. The cleavage specificity of pepsin is promiscuous, hindering rational engineering of pepsin-stable protein scaffolds.

Mouse or chicken gastric fluid was compared to 1 mg/mL pig pepsin for degradation of monomeric enhanced green fluorescent protein (mEGFP) (3,028 U/mL) (FIG. 1A). In all conditions tested, ˜50% mEGFP signal was lost within 10 s, with comparable and rapid digestion (FIG. 1A). Therefore, 1 mg/mL pig pepsin pH 2.2 was set as the benchmark that a scaffold for gastric applications should resist, with pepsin activity maximal at pH 1.5-2.5.

Numerous non-antibody protein scaffolds for imaging and therapy are under clinical development. However, rarely are the scaffolds tested for stability in GI tract-like conditions. Nanofitins are scaffolds engineered from Sac7d, a DNA-binding protein from an acidophilic archaeon, in part for oral administration. Nanofitins have been reported to be stable at low pH and in the presence of pepsin. Heavy chain single-domain antibody fragments (nanobody) are a class of small (12-15 kDa) protein scaffolds that express well in microbial culture and bind targets with comparable affinity to antibodies. Nanobodies normally have low stability to GI tract conditions, but have been engineered to have a second disulfide bond, to improve protease-resistance. Anti-IgG-Sso7d (nanofitin) and the nanobody engineered for special pepsin-resistance were expressed in E. coli. Kunitz-type soybean trypsin inhibitor (SBTI) was expressed in E. coli T7Shuffle (enabling efficient disulfide bond formation in the cytosol) and purified SBTI via a His₆-tag using Ni-NTA. Protein yields were up to 2.9 mg/L culture. Electrospray ionization mass spectrometry (ESI-MS) confirmed the identity of the expressed SBTI and that the two disulfide bonds had been formed.

After recombinant expression and purification, the proteins were incubated with pepsin. The nanofitin and nanobody were incubated with serial dilutions of 1 mg/mL pepsin for 10 min at 37° C. at pH 2.2 (FIG. 1B). Neither the nanofitin nor the nanobody was detectable by Coomassie staining after 10 min in the presence of the benchmark pepsin concentration (1 mg/ml, 3,028 U/mL) (FIG. 1B). The pepsin-resistance of SBTI was tested and found to be remarkably stable to the digestion tests (FIG. 1B). In contrast to the nanobody and nanofitin, little to no degradation of SBTI was observed after 10 min in the presence of 1 mg/mL pepsin (FIG. 1B). In fact, even with 100-fold dilution of pepsin, the nanofitin and nanobody were almost completely degraded (FIG. 1B).

### Example 2-Assessment of the Stability of SBTI

It was hypothesized that SBTI may be a promising scaffold candidate for the gastric environment.

Bile acids play an important role in intestinal absorption of lipids, but also potentiate the activity of digestive proteases. Post-prandial free bile acid concentrations in the human small intestine reach up to 10 mM. To investigate the effect of bile acids on the native activity of SBTI, SBTI was pre-incubated with 10 mM of the most abundant bile acids in human bile, before testing SBTI's trypsin inhibition activity. It was observed that the bile acids increased trypsin activity, but SBTI maintained efficient inhibition of trypsin in the presence of each of the bile acids (FIG. 2A-E).

Pancreatin is secreted from exocrine cells of the pancreas and includes the proteases trypsin, chymotrypsin and elastase, which are the main endopeptidases in the intestine. The intestinal stability of the nanofitin and nanobody described in Example 1 and SBTI was tested by incubating each in serial dilutions of pancreatin for 30 min (FIG. 3A). No digestion of SBTI was observed after 30 min in the presence of intestine-like concentrations of pancreatin (10 mg/mL) (FIG. 3B). The nanobody and nanofitin, however, were not detectable after 30 min in the presence of 10-fold lower concentration of pancreatin (1 mg/mL) (FIG. 3A).

Clinically approved anti-TNF-α monoclonal antibodies Adalimumab and Infliximab are digested by elastase in simulated intestinal conditions. SBTI and the nanofitin and nanobody described in Example 1 were incubated with serial dilutions of elastase for 30 min (FIG. 3B). At elastase concentrations representative of the small intestine (10 U/mL), nanobody and nanofitin were fully digested. With elastase, SBTI underwent only a small change in molecular weight, consistent with removal of its His6-tag (FIG. 3B). Therefore, SBTI shows higher stability than these leading ligand binding proteins to intestinal proteases.

Thermal resilience is an important characteristic for applications of proteins, for example in animal feed supplementation or in facilitating modification and evolution. The thermal resilience of SBTI was tested by heating at various temperatures from 75 to 100° C. for 10 min (FIG. 4A). Aggregated protein was pelleted by centrifugation and the soluble protein determined by SDS-PAGE. As expected, β-lactamase (BLA), used as an example of a typical mesophilic protein, was almost completely aggregated by 75° C. incubation (FIG. 4A,B). On the other hand, heating SBTI to 100° C. for 10 min led to >90% retention of solubility (FIG. 4A,B), showing its excellent thermal resilience.

Most proteins are easily denatured by acidic conditions. To determine the thermal unfolding transition of SBTI at neutral or gastric pH, differential scanning calorimetry (DSC) was performed (FIG. 4B). At pH 7.4 a Tₘ of 67.2° C. for SBTI was observed, whilst the Tₘ of SBTI only underwent a minor shift to 60.3° C. at pH 2.0 (FIG. 4B). These data indicate that SBTI retains high stability in the extreme low pH environment characteristic of the stomach.

### Example 3-Development of SBTI as a Ligand-Binding Protein

The evolvability of SBTI as scaffold protein (i.e. a potential ligand binding protein) was investigated. Rosetta modelling software was used to guide experimental work. The objective was to identify continuous amino acid stretches that, when mutated, did not substantially reduce the stability of the protein.

As a first step, an ensemble of structures of SBTI was generated based on the crystal structure, using Rosetta's relax function, before analyzing a pool of 467 structures. Since the objective was to evolve SBTI to bind other proteins, the mutability of solvent-accessible residues was investigated. The solvent-accessible surface area (SASA) of a representative SBTI structure was calculated using the Parameter Optimised Surfaces (POPS) webserver. Solvent-accessible residues were mutated to each of the other 20 amino acids except cysteine, using Rosetta's pmutscan function. Cysteine was omitted to avoid potential dimerization or interference with existing disulfide bonds. Mean changes in Rosetta Energy Units (ΔREU) at each residue were visualized in PyMOL. Mutations to proline were excluded in calculations of mean ΔREU because they were extremely destabilizing. This analysis identified two suitable amino acid loops, which were termed gastrobody determining regions (GDR). GDR1 comprises D22, 123, T24 and A25, i.e. residues 22-25 of SEQ ID NO: 1. GDR2 comprises R47, N48, E49 and L50, i.e. residues 47-50 of SEQ ID NO: 1.

Building on this computational analysis, alanine-scanning mutagenesis was performed: each residue in GDR1/2 was individually mutated to alanine (except A25 which was mutated to glycine). GDR1/2 alanine mutants were then subjected to tests of heat-resilience (FIG. 5A) and pepsin-resistance (FIG. 5B). As before, heat-resilience was measured by loss of soluble protein after 10 min at high temperatures (75-100° C.), with BLA as a positive control thermolabile protein. None of the SBTI alanine mutants in GDR2 led to substantial loss of heat resilience relative to wild-type (WT) SBTI (FIG. 5A). SBTI D22A and T24A in GDR1 exhibited a decrease in heat-resilience but still a substantial fraction of each mutant was resilient to 90 and 100° C. (FIG. 5A).

To test pepsin-resistance, GDR1/2 alanine mutants were incubated with 1 mg/mL pepsin at pH 2.2 for 100 min at 37° C. (FIG. 5B). These mutants retained good pepsin-resistance. 40% of the most susceptible mutant (N48A) remained after 100 min in the presence of 1 mg/mL pepsin, compared to 80% of WT SBTI. In fact, some mutants showed pepsin-resistance better than WT SBTI, such as D22A (95% at 100 min) or L50A (91% at 100 min) (FIG. 5B). Overall, SBTI was able to tolerate mutations through GDR1 and 2.

### Example 4—Screening Phage Display Libraries Containing SBTI Mutants

SBTI and its mutants were displayed on M13 at the N-terminus of minor coat protein pIII. Each M13 phage particle contains five copies of pIII.

Domains of toxin A (TcdA) and toxin B (TcdB) of C. difficile were selected as targets for the identification of SBTI mutants with selective ligand-binding properties. Globally there are 2.2 cases of healthcare facility-associated incidences of infection with the Gram-positive bacterium C. difficile per 1000 admissions per year. Key effectors in C. difficile pathogenesis are the toxins TcdA and TcdB, disrupting the intestinal epithelium. Passive immunization against the toxins protects against C. difficile challenge. Actoxumab (anti-TcdA) and bezlotoxumab (anti-TcdB) are fully human neutralizing antibodies that have been evaluated in phase III clinical trials for prevention of recurring C. difficile infection by intravenous administration. Bezlotoxumab was subsequently approved by the Food and Drug Administration (FDA).

First, phage display was used to select SBTI ligand binding polypeptides (termed “gastrobodies”) with four randomized residues in both GDR1 and GDR2 for binding to the combined repetitive oligopeptide domain of C. difficile toxin A (residues 2304-2710, CROP). After performing three rounds of selection, ten clones were sequenced (Table 3), which had been screened for CROP binding using a monoclonal phage ELISA.

TABLE 3
Loop sequences of anti-CROP hits.
10 hits from anti-CROP selections
were sequenced. Frameshift mutations
are indicated by ‡. Amber codons
(TAG), repressed as Gln in TG1
cells, are indicated by *

| | Sequence | | |
|---|---|---|---|
| | GDR1 | GDR2 | |
| | (SEQ ID | (SEQ ID | |
| Clone | NO:) | NO:) | Elution |
| WT | DITA (26) | RNEL (27) | n/a |
| G2 | IAGT (28) | LVFP (29) | Glycine |
| G9 | HPDL (30) | LYLF (31) | Glycine |
| G22 | L*NR (32) | HDFM (33) | Glycine |
| G27 | IAGT (28) | LVFP (29) | Glycine |
| G28^{‡} | PYRY (34) | WLHR (35) | Glycine |
| T12 | IAGT (28) | LVFP (29) | TEA |
| T18 | VPH* (36) | RNKW (37) | TEA |
| T23^{‡} | SFRG (38) | FRLE (39) | TEA |
| T29^{‡} | RPPQ (40) | TWGW (41) | TEA |
| T42 | VPH* (36) | RNKW (37) | TEA |

^{‡}frameshift mutation
*amber stop codon

Anti-CROP gastrobodies were cloned into pET28a and expressed in E. coli T7SHuffle. The stability of selected gastrobodies was assessed by DSC at pH 7.4 and pH 2.0 (FIG. 6B and Table 4).

TABLE 4
Summary of anti-CROP clone melting
temperature and affinity for CROP

| | | | CROP |
|---|---|---|---|
| | Tₘ (° C.) from DSC | | binding |
| Clone | pH 7.4 | pH 2.0 | K_{d} (μM) |
| WT | 67.3 | 60.4 | — |
| G9 | 69.9 | 55.2 | 4.6 |
| G22 | 75.3 | 61.0 | ~50 |
| T12 | 66.8 | 53.6 | 1.7 |
| T42 | 78.6 | 71.0 | 7.3 |

Binding to CROP was confirmed by surface plasmon resonance (SPR) (FIG. 6A and Table 4). At pH 7.4, two anti-CROP gastrobodies had a Tₘ similar to WT SBTI, while two gastrobodies showed substantially higher Tₘ (FIG. 6B). The Tₘ of anti-CROP gastrobodies at pH 2.0 shifted both higher and lower than WT (FIG. 6B and Table 4). The K_{d} of binders observed was in the single-digit micromolar range (FIG. 6A and Table 4). Importantly, anti-CROP gastrobodies retained the high pepsin stability of WT SBTI, although the terminal His6-SpyTag003 tail was rapidly removed (FIG. 6C).

For the second target, the glucosyltransferase domain of TcdB (GTD) was chosen. It was hypothesized that increasing the number of randomized residues in GDR1/2 would improve the affinity of binders but could also be more susceptible to pepsin cleavage. Therefore, a new gastrobody library was cloned which randomized five, six or seven residues in each GDR. After optimizing the Gibson cloning and competent cell electroporation, phage library sizes of ˜10⁹ we achieved. The gastrobody library with extended loops was displayed on M13. Rounds of selection were performed against biotinylated AviTag-His6-GTD. In later rounds, incubation was performed with excess non-biotinylated bait to promote the selection of low off-rate variants. Incubation of the amplified phage library was tested with 0.1 mg/mL pepsin at pH 2.2 at 37° C. for 10 min, before incubating with biotinylated AviTag-His6-GTD, to favour the selection of gastrobodies retaining pepsin-resistance. All selected clones featured an amber codon (TAG), which is suppressed by a glutamine (Gln, Q) in TG1 cells. The length of both GDR1 and GDR2 was six residues in our optimal binder (GT01) (Table 5).

TABLE 5
Loop sequence in anti-GTD gastrobody
GT01 at GDR1 and GDR2,
compared to WT SBTI

| | GDR1 | GDR2 |
|---|---|---|
| Clone | (SEQ ID NO:) | (SEQ ID NO:) |
| WT | DITA (26) | RNEL (27) |
| GT01 | DYGRQL (42) | LERNHR (43) |

The GT01 gene from the screen was cloned into pET28a, the amber codon was corrected, and expressed in T7Shuffle. GT01 was purified by Ni-NTA and then size exclusion chromatography. The identity of the protein and formation of the disulfide bonds was confirmed by ESI-MS. The binding kinetics were analyzed by SPR (FIG. 7B, showing an on-rate of 4.2±0.3×10⁵ M⁻¹s⁻¹ and an off-rate of 3.6±0.2×10⁻² s⁻¹. This revealed a dissociation constant (K_{d}) in the nanomolar range (85±2.3 nM). The pepsin stability of GT01 was assessed by incubating for 30 min in 1 mg/mL pepsin at pH 2.2 at 37° C. Under these harsh conditions, more GT01 was degraded than WT SBTI, but there was still substantial intact gastrobody after 30 min (FIG. 7A).

TcdB toxin delivers the GTD domain into the cytoplasm of epithelial cells. In the cytoplasm, GTD glucosylates Rho GTPases, which disrupts the cytoskeleton, leading to cell death and compromise to the epithelial barrier of the intestine (FIG. 7C). In the absence of an acceptor protein, GTD hydrolyzes UDP-glucose. The gastrobody was only selected for binding to TcdB, but it was determined whether the gastrobody had any effect on GTD catalytic activity. The hydrolytic activity of GTD was assayed by incubating the protein with UDP-glucose and detecting free UDP (FIG. 7C). GT01 indeed was able to inhibit GTD in a dose-dependent manner, with the WT SBTI negative control showing no effect on GTD activity (FIG. 7D). Thus gastrobodies can be selected to bind disease-relevant proteins with nanomolar affinity and also targeted to inhibit enzyme activity.

### Example 5-Analysis of Gastrobody Binding Specificity

GT01 and T12 (so-called “gastrobodies”) were tested using an ELISA to determine whether they bind to their respective targets specifically.

Wells of a 96-well Nunc Maxisorp (44-2404-21, Thermo Fisher) were coated with 5 μg/mL antigen HEL, BLA, trypsin, GTD or CROP in PBS pH 7.4 overnight at 4° C. Antigen-coated wells were washed once with PBS-T and blocked with 5% skim milk in PBS pH 7.4 for 2 h at RT. 500 nM gastrobody in PBS pH 7.4 was incubated in wells for 30 min at RT. Bound gastrobody was detected with primary antibody 1:5,000 rabbit anti-trypsin inhibitor (34549, Abcam) and secondary antibody 1:7,000 anti-rabbit IgG (H+L): HRP (65-6120, Invitrogen) in 1% skim milk in PBS-T. Antibodies were allowed to bind for 45 min at RT. Between each incubation, wells were washed three times with PBS-T. After three final washes the ELISA was developed with 1-Step Ultra TMB-ELISA substrate solution (34029, Thermo Fisher). Colour change was monitored using a FLUOStar Omega (BMG Labtech) at 652 nm.

FIG. 8 shows that GT01 bound both trypsin and GTD but not the control antigens CROP, BLA and HEL. Similarly, T12 was observed as binding to CROP and trypsin but not the control antigens, including GTD.

### Example 6-Identification and Analysis of Additional Candidate GDRs in SBTI

The computation analysis described in Example 3 identified two additional candidate GDRs in SBTI. GDR3 comprises N6, E7, G8 and N9, i.e. residues 6-9 of SEQ ID NO: 1. GDR4 comprises S124, D125, D126, E127 and F128, i.e. residues 124-128 of SEQ ID NO: 1. GDR3 (N6-N9) had a favourable mutability score (1.3) but was missing a mean ΔREU score for G8 because the residue was not solvent accessible. GDR4 (S124-F128) consists of five consecutive residues but the mean ΔREU was 3.0, above the criteria of <2.0. The variance of the mean ΔREU of GDR4, however, was high: AREU of D126 was 10 while the remaining four residues were below 2.0. GDR3/4 are close to GDR1/2 in folded protein.

### Pepsin Resistance of GDR3/4 Alanine Mutants

Alanine mutants in GDR3/4 were generated and found to be stable in the presence of 1 mg/mL pepsin. The alanine mutants were incubated with 1 mg/mL pepsin for 30 min at pH 2.2 and 37° C., before measuring intact protein by SDS-PAGE with Coomassie staining (FIG. 9). D125A was the most susceptible mutant with 33% remaining after 30 min, compared to 79% of WT SBTI, or 97% of the most stable mutant (S124A). Alanine mutants in GDR4 were more stable than mutants in GDR3 (FIG. 9).

### Example 7-Phage Display for Affinity Maturation of GT01 with Randomised GDR3/4

As shown in Example 6, mutating individual residues in GDR3/4 to alanine did not lead to complete loss of pepsin-resistance. Accordingly, the ability of GDR3 and GDR4 to improve the affinity of gastrobody GT01 was assessed by phage display.

A new naïve library was generated with NNK-randomised GDR3/4, using GT01 fused to full-length pIII as template. Any combination of four or five residues in GDR3 and five, seven or nine residues in GDR4 were randomised. The library contained 3.3×10⁸ variants and construction was validated by sequencing ten clones.

Binders against GTD were selected through four rounds of affinity maturation, with increasing stringency each round. In the final round the bait concentration was 1 nM, the on-phase 10 min, and included three 1 h off-rate washes with excess non biotinylated bait at 37° C. After four rounds of affinity maturation selection with the GDR3/4 library against biotinylated GTD, nine clones were sequenced (Table 6). Two clones had the WT GDR3 sequence (NEGN, SEQ ID NO: 63) and all clones had Gly in position three at GDR3. Four of the nine clones had Arg in position four of GDR3. All the hit GDR3 sequences consisted of four amino acids-no five amino acid GDR3 was found (Table 6). Clones with five, seven and nine amino acids in GDR4 were identified. No obvious consensus motif emerged in the selections (Table 6).

TABLE 6
Loop sequences in anti-GTD
gastrobodies at GDR3 and
GDR4, compared to WT SBTI

| Clone | GDR3 | GDR4 |
|---|---|---|
| # | (SEQ ID NO:) | (SEQ ID NO:) |
| WT | NEGN (63) | SDDEF (64) |
| 1 | NEGN (63) | RKTRA (65) |
| 2 | TSGR (66) | SHGRLRTVA (67) |
| 3 | VDGS (68) | WPTNTGF (69) |
| 4 | NEGN (63) | NPSNG (70) |
| 5 | DQGR (71) | NTLWGSR (72) |
| 6 | MDGA (73) | DVNFQ (74) |
| 7 | TQGG (75) | RPTSSTG (76) |
| 8 | IKGR (77) | PTGAG (78) |
| 9 | TGGR (79) | RRRDR (80) |

### Example 8—Analysis of Gastrobodies with Mutated GDRs 1, 2 and 4

The GDR4 sequences of clones 4 and 7 from Example 7 were incorporated into the GT01 gastrobody to produce GT44 and GT47, respectively (Table 7).

TABLE 7
Loop sequences in anti-GTD
gastrobodies at GDR1-4
compared to WT SBTI

| Pro- | GDR1 | GDR2 | GDR3 | GDR4 |
|---|---|---|---|---|
| tein | (SEQ ID | (SEQ ID | (SEQ ID | (SEQ ID |
| ID | NO:) | NO:) | NO:) | NO:) |
| SBTI | DITA (26) | RNEL (27) | NEGN (63) | SDDEF (64) |
| (WT) | | | | |
| GT01 | DYGRQL (42) | LFRNHR (43) | NEGN (63) | SDDEF (64) |
| GT44 | DYGRQL (42) | LFRNHR (43) | NEGN (63) | NPSNG (70) |
| GT47 | DYGRQL (42) | LFRNHR (43) | NEGN (63) | RPTSSTG |
| | | | | (76) |

After expression and purification by Ni-NTA and SEC, the binding kinetics of GT44 and GT47 to immobilised GTD were analysed by SPR. The affinity of GT47 (Kd=10.3 nM) was approximately 8-fold better than GT01 (Kd=85±2.3 nM). Driven by improvements of on- and off-rate, both GT44 and GT47 bound more tightly than GT01 (Table 8).

TABLE 8
Gastrobody binding affinity properties

| Pro- | | | |
|---|---|---|---|
| tein | | | Kd |
| ID | kₒₙ (M⁻¹s⁻¹) | k_{off} (s⁻¹) | (nM) |
| GT01 | (4.2 ± 0.3) × 10⁵ | (3.6 ± 0.2) × 10⁻² | 85 ± 2.3 |
| GT44 | 1.3 × 10⁶ | 2.2 × 10⁻² | 17.4 |
| GT47 | 2.1 × 10⁶ | 2.2 × 10⁻² | 10.3 |

The heat resilience of GT44 and GT47 was tested with an ELISA. Binding to GTD was analysed after heating to 37, 55, 75 or 100° C. for 10 min. Both GT44 and GT47 showed minimal loss of GTD-bound protein after heating to 55° C. compared to 37° C. However, GT47 was more to susceptible heat-induced loss of binding than GT44 when heating to 75° C.

GT44 and GT47 were also subjected to a test of pepsin-resistance in which the anti-GTD gastrobodies were incubated with 1 mg/mL pepsin at pH 2.2 and 37° C. for 30 min, before neutralising and measuring binding at pH 7.4. 3-fold less GT44 and GT47 bound GTD after 30 min with 1 mg/mL pepsin, consistent with findings for GT01.

### Example 9—Removing Trypsin Binding of GT01

As the trypsin inhibition activity of SBTI family polypeptides might be detrimental to some of the applications in which gastrobodies may find utility, the removal of this activity may be desirable. The scissile R63 of SBTI is a key residue for trypsin binding and the GDRs are on the opposite face of SBTI to R63.

A mutant GT01 protein comprising the substitution R63A was generated (GT01 R63A) to determine whether this is sufficient to remove trypsin binding of GT01.

GT01 R63A and GT01 were compared for trypsin binding. GT01 bound to trypsin with an apparent dissociation constant in the nanomolar range, but no binding of GT01 R63A to trypsin was observed (FIG. 10A). The binding kinetics of GT01 R63A to GTD were determined to be 78.7±21.5 nM, which was not substantially different from the Kd of GT01 binding to GTD (85±2.3 nM). Consistent with the SPR analysis, no difference in apparent binding affinity was observed in an ELISA comparing GT01 and GT01 R63 binding to GTD (FIG. 10B).

To determine the impact of the R63A mutation on the protease stability of GT01, GT01 R63A was pre-incubated with physiological concentrations of pepsin (1 mg/mL) at pH 2.2, or with chymotrypsin (25 U/mL) or elastase (10 U/mL) at pH 6.8 and 37° C. for 30 min before testing binding to GTD at pH 7.4. After incubation with each protease, approximately 3-fold less GT01 R63A bound to GTD compared to a control kept in PBS. GT01 R63A was most susceptible to trypsin of all the proteases tested: pre-incubation with intestinal concentrations of trypsin (100 U/mL) for 30 min at pH 6.8 and 37° C. caused 4-fold loss of bound GT01 R63A compared to the 0 min time point. Pre-incubation with 10 U/mL trypsin for 30 min reduced bound GT01 R63A 3-fold compared to the 0 min time point.

### Example 10—Analysis of Alternative Gastrobody Scaffolds

The pepsin-, pancreatin- and heat-resilience of structural homologues of SBTI was investigated. PDBe Fold was used to identify three structural homologues of SBTI: the Erythrina caffra seed Kunitz-type trypsin inhibitor (ECTI, PDB ID: 1TIE); the Bauhinia bauhinioides plasma kallikrein inhibitor (BBKI, PDB ID: 4ZOT, previously known as BBTI), and the winged bean chymotrypsin inhibitor (WBCI, PDB ID: 1EYL). The sequence identity of SBTI to ECTI is 42%, 29% to BBKI, and 44% to WBCI. ECTI and WBCI have two disulfide bonds in analogous positions to SBTI. BBKI has one unpaired cysteine. A mutant version of the BBKI polypeptide was generated in which the unpaired cysteine residue was substituted with alanine (SEQ ID NO: 85, encoded by SEQ ID NO: 86). The mutant BBKI polypeptide was termed BBKICA.

The pepsin stability of BBKI, ECTI, WBCI and BBKICA was tested with the benchmark challenge with 1 mg/mL pepsin at pH 2.2 and 37° C. In this preliminary test of pepsin stability, the structural homologues were more resistant to digestion than SBTI. BBKICA showed a similar level of resistance to BBKI. WBCI was the most stable with 91% remaining after 120 min with 1 mg/mL pepsin, compared to 59% of SBTI (FIG. 11A).

Similarly to SBTI (Example 2), BBKI and BBKICA were largely unaffected by pancreatin after 30 minutes. WBCI was shown to be less resistant to pancreatin than SBTI, but a substantial amount remained after 30 min of digestion with pancreatin at 1 mg/ml. ECTI was found to be fully digested by 0.1 mg/mL pancreatin after 30 minutes.

SBTI, ECTI, WBCI, BBKI and BBKICA were heated to 75, 90 or 100° C. for 10 min to investigate the heat-induced aggregation tendency of the proteins. After heating, aggregates were pelleted, and the soluble fraction of the protein monomer was quantified by SDS-PAGE with Coomassie staining and gel densitometry (Table 9). SBTI and ECTI remained almost completely soluble after 10 min at 100° C. (FIG. 11 C,F,G). Substantial fractions of soluble monomer of BBKI and WBCI disappeared upon heating above 75° C. (FIG. 11 D,E,G). Bands indicative of dimer formation were observed for BBKI at all temperatures, increasing in intensity after heating to 100° C. (FIG. 11E). WBCI formed dimers and oligomers after heating to 90 or 100° C. (FIG. 11D).

No bands indicative of dimers were observed for BBKICA (BBKICA was monomeric throughout the temperature range, FIG. 11B), indicating that the cysteine residue in BBKI is responsible for dimer formation. Moreover, a greater proportion of BBKICA was soluble at 90 and 100° C. in comparison to BBKI, indicating that substitution of the cysteine residue improved the thermal resilience of BBKI.

TABLE 9
Solubility of gastrobody scaffolds after heating

| | Soluble fraction (%) | | | | |
|---|---|---|---|---|---|
| Temperature (° C.) | SBTI | WBCI | ECTI | BBKI | BBKICA |
| 25 | 100 | 100 | 100 | 100 | 100 |
| 75 | 104 | 94 | 104 | 99 | 97 |
| 90 | 84 | 53 | 103 | 16 | 23 |
| 100 | 86 | 60 | 100 | 39 | 80 |

Following the aggregation tests, DSC was used to compare the melting temperatures of SBTI, WBCI, ECTI and BBKI at pH 7.4 and at pH 2.0. All structural homologues were more stable than SBTI at both pH 7.4 and pH 2.0. BBKI had the highest melting temperature at pH 7.4 (˜86° C.) followed by WBCI (˜84° C.). However, BBKI was also most destabilized by acid, with the Tm shifting by 20° C. to 68° C. at pH 2.0. The Tm of ECTI, by contrast, only shifted 7° C. from 79° C. at pH 7.4 to 72° C. at pH 2.0.

### Experimental Procedures

### Plasmids and Cloning

Standard PCR methods and Gibson assembly were used to clone constructs. All inserts were validated by Sanger sequencing. Codon-optimized sequences of SBTI, anti-CDTA nanobody protein 4.2m, nanofitin anti-IgG Sso7d, C. difficile toxin B glucosyltransferase domain (GTD, residues 2-543) were ordered as gBlocks® (Integrated DNA Technologies) and cloned into pET28a. GTD was cloned in the format pET28a-AviTag-His6-GTD to enable site-specific biotinylation. Similarly, CROP was cloned in the format pET28a-AviTag-His₆-CROP. WT and alanine mutants of SBTI were in the format pET28a-SBTI-His₆. Gastrobody hits from selections were cloned in the format pET28a-His₆-Thrombin site-SpyTag003-SBTI derived from pET28a-SpyTag003-MBP (Addgene plasmid ID 133450). Point mutations were made by Gibson assembly. The phagemid vector pBAD-DsbA (ss)-SBTI-pIII (216-425) was constructed from pFab5c and MP6 (Addgene plasmid ID 69669, a kind gift from David Liu). pET28a-His₆-Thrombin site-mEGFP was generated by Dr. Robert Wieduwild in the Howarth group.

The pBAD-DsbA (ss)-SBTI-pIII (216-425) libraries were constructed by Gibson assembly from PCR products made with degenerate oligonucleotides (Integrated DNA Technologies) with NNK codons for randomized residues (SBTI residues 22, 23, 24, 25, 47, 48, 49, 50 and insertions to increase binding loop length). Separate assembly reactions were set up for each combination of GDR loop size. 0.2 pmol of each PCR fragment was combined with NEBuilder HiFi DNA Assembly Master Mix (NEB) in a final volume of 20 UL and incubated for 2 h at 50° C. Assembly reactions were pooled and purified using the Wizard SV Gel and PCR clean up kit (Promega). Purified assembled phagemid DNA was eluted in MilliQ water. Eight aliquots of 25 μL electrocompetent E. coli TG1 (Lucigen) were transformed with 300 ng of library DNA. Electroporations were performed in 0.2 mm cuvettes (Bio-Rad) with a MicroPulser (165-2100, Bio-Rad) delivering a single 2.5 kV pulse. Each electroporation was immediately recovered in 1 mL recovery medium (Lucigen) and incubated for 1 h at 37° C., 200 RPM. Recovered cells were pooled and plated onto LB+0.8% (w/v) glucose+100 μg/mL carbenicillin, and grown for 16 h at 37° C. Cells were resuspended in 2×TY and pelleted by centrifugation at 16,900 g for 15 min at 4° C. Library cell pellets were resuspended in 2×TY+20% (v/v) glycerol and stored at −80° C.

### Protein Expression

For expression of SBTI, chemically-competent E. coli T7 SHuffle® (NEB) (to facilitate formation of disulfide bonds in the cytosol) was transformed with pET28a-SBTI-His₆ or pET28a-His₆-Thrombin site-SpyTag003-SBTI or mutants thereof. E. coli BL21 (DE3) RIPL (Agilent) was transformed with pET28a-anti-CROP-His₆ (nanobody), pET28a-anti-IgG-Sso7d-His₆ (nanofitin), pET28a-AviTag-His₆-CROP, PET28a-His₆-Thrombin site-mEGFP. E. coli T7 Express lys Y/I^{q} (NEB) were transformed with pET28a-AviTag-His₆-GTD. Transformants were plated onto lysogeny broth (LB) agar plates containing 50 μg/mL kanamycin and grown overnight at 37° C. Single colonies were inoculated into 10 mL LB+50 μg/mL kanamycin and grown for 16 h at 37° C. at 200 RPM for use as starter cultures. 1 mL of starter culture (except nanofitin and His₆-Thrombin site-SpyTag003-SBTI or mutants thereof) was inoculated into 200 mL or 1 L auto-induction medium (AIMLB0205, Formedium) containing 50 μg/mL kanamycin. mEGFP and pET28a-anti-CROP were grown for 22 h at 30° C. at 200 RPM. pET28a-AviTag-His₆-CROP was grown at 37° C. at 200 RPM until OD₆₀₀=0.1, before continuing growth for 18 h at 25° C., 200 RPM. pET28a-SBTI-His₆ was grown at 37° C. at 200 RPM for 6 h, followed by 16 h at 18° C. at 200 RPM. A starter culture of nanofitin or His₆-Thrombin site-SpyTag003-SBTI or mutants thereof was inoculated into 1 L LB (nanofitin, His₆-Thrombin site-SpyTag003-anti-CROP gastrobodies) or 2×TY+0.5% (v/v) glycerol (His₆-Thrombin site-SpyTag003-GT01 or His₆-Thrombin site-SpyTag003-SBTI) with 50 μg/mL kanamycin and grown at 37° C. at 200 RPM until A₆₀₀=0.5. Expression was induced by addition of isopropyl β-D-1-thiogalactopyranoside (IPTG) (Fluorochem) to 0.42 mM and grown for 16 h at 30° C. (nanofitin) or 18° C. (His₆-Thrombin site-SpyTag003-SBTI) at 200 RPM. All cultures were harvested by centrifugation at 4,000 g for 15 min at 4° C.

### Protein Purification

Proteins were purified using Ni-NTA. Purification was performed at 4° C. Cell pellets were resuspended in PBS (137 mM NaCl, 2.7 mM KCl, 10 mM Na₂HPO₄, 1.8 mM KH₂PO₄, PH 7.4) and centrifuged for 15 min at 4,000 g. The supernatant was discarded. Washed cell pellets of nanobody, nanofitin, AviTag-His₆-CROP, AviTag-His₆-GTD, mEGFP, His₆-Thrombin site-SpyTag003-GT01, or His₆-Thrombin site-SpyTag003-SBTI were resuspended in 1×Ni-NTA buffer (50 mM Tris-HCl, 300 mM NaCl, pH 7.8) supplemented with complete Mini EDTA-free Protease Inhibitor Cocktail and 1 mM phenylmethylsulfonyl fluoride (PMSF). Cell lysis was initiated by addition of 100 μg/mL lysozyme (Merck) and incubation at 25° C. for 30 min. Lysate was sonicated on ice four times for 1 min, with 1 min rest period at 50% duty cycle.

Washed pellets of SBTI-His₆, His₆-Thrombin site-SpyTag003-anti-CROP gastrobodies were resuspended in BugBuster 10× protein extraction reagent (Merck) supplemented with 2 U/mL benzonase, 100 g/mL lysozyme, complete Mini EDTA-free Protease Inhibitor Cocktail and 1 mM PMSF. Cells were incubated for 30 min at 25° C. on a roller for complete lysis. 2-mercaptoethanol was added to 10 mM prior to clarification of lysate.

Cell lysates were cleared by centrifugation at 16,900 g for 30 min at 4° C. Clarified lysate was incubated with Ni-NTA beads (Qiagen) on a rotary shaker for 45 min before transferring to a Polyprep gravity column. Beads were washed with 10 packed-resin volumes of Ni-NTA buffer+10 mM imidazole (wash 1). For SBTI-His₆ or His₆-Thrombin site-SpyTag003-anti-CROP gastrobodies, 10 mM 2-mercaptoethanol was included in the first 10 column volumes of wash 1. Following wash 1, beads were washed with 5 column volumes of Ni-NTA buffer with 30 mM imidazole. Proteins were eluted with Ni-NTA buffer with 200 mM imidazole. A280 Of elutions was monitored and fractions were pooled for dialysis. SBTI-His₆, His₆-Thrombin site-SpyTag003-anti-CROP gastrobodies were dialyzed against 50 mM Tris-HCl PH 8.0+100 mM NaCl. Protein concentration was determined from A280 using a NanoDrop and the extinction coefficient predicted by ExPASy ProtParam. Nanobody, nanofitin, AviTag-His₆-CROP, AviTag-His₆-GTD, mEGFP or His₆-Thrombin site-SpyTag003-anti-GTD gastrobody and His₆-Thrombin site-SpyTag003-SBTI were dialyzed against PBS.

His₆-Thrombin site-SpyTag003-SBTI, His₆-Thrombin site-SpyTag003-gastrobodies and AviTag-His₆-GTD (post-biotinylation) were purified by gel filtration. Proteins were concentrated to <1 mL using Vivaspin 6 10 kDa MWCO (Sartorius) spin columns and loaded onto a HiLoad 16/600 Superdex 75 pg column connected to an ÅKTA Pure 25 (GE Healthcare) at 1 mL/min flow rate of PBS at 4° C. AviTag-His₆-GTD was run on a HiLoad 16/600 Superdex 200 pg column. Elutions were pooled and concentrated using Vivaspin 20 5 kDa MWCO (Sartorius).

Typical protein yields per L culture were 23 mg for nanobody, 12 mg for nanofitin, 2 mg for AviTag-His₆-CROP, 3 mg for SBTI, 0.5-2 mg for SBTI variants, 1.8 mg for AviTag-His₆-GTD and 30 mg for mEGFP.

BLA-His₆ was a kind gift from Jisoo Jean in the Howarth Group. BLA-His₆ was expressed in E. coli BL21 DE (3) RIPL in LB with 0.8% glucose at 18° C. overnight, purified with Ni-NTA (Qiagen) using standard methods and dialyzed into PBS pH 7.4.

### SDS-PAGE and Image Analysis

Samples were mixed with 5×SDS-PAGE loading buffer [0.23 M Tris HCl pH 6.8, 24% (v/v) glycerol, 120 μM bromophenol blue, 0.23 M sodium dodecyl sulfate, 100 mM 2-mercaptoethanol], heated for 3 min at 99° C., and loaded onto a 16% Tris-glycine gel. Gels were run in an XCell SureLock system (Thermo Fisher Scientific) for 60 min at 190 V, stained with InstantBlue Coomassie stain (Expedeon), destained with MilliQ water, and imaged using a ChemiDoc XRS imager. Gel densitometry was performed with ImageLab 6.0.1 (Bio-Rad).

### Gastric Fluid Proteolysis Assay

Female non-medicated, non-immunized, non-fasted BALB/c mouse gastric fluid was purchased from BioIVT. Chicken gastric fluid was collected post-mortem from gizzards of ad libitum-fed day 22 Ross 308 broilers at Drayton Animal Health (UK). Chicken gastric fluid was clarified by centrifugation at 16,900 g for 30 min at 4° C. 14 μM mEGFP (final concentration) was incubated with 1 mg/mL pepsin from pig gastric mucosa (P6887, Merck) (final concentration 3,028 U/mL) in 50 mM glycine-HCl pH 2.2, or mouse or chicken gastric fluid at 37° C. Digestion was stopped by addition of 1 M Tris pH 8.8, incubating for 5 min at 25° C. to allow mEGFP to re-gain fluorescence, and measuring fluorescence at 528 nm (excitation 488 nm) with a ClarioSTAR plate-reader (BMG Labtech). Fluorescence at t=0 was set to 100%. For t=0 wells, 1 M Tris pH 8.8 was added to inactivate pepsin before addition of mEGFP.

### Trypsin Inhibition Assay

80 U/mL trypsin from cow pancreas (T1426, Merck) was incubated with serial dilutions of SBTI in 50 mM Tris-HCl PH 8.0, along with 10 mM of the individual bile acids sodium glycocholate hydrate (G7132, Merck), sodium glycodeoxycholate (G9910, Merck), sodium taurocholate hydrate (86339, Merck) or sodium taurodeoxycholate hydrate (T0875, Merck) for 20 min at 25° C. The reaction was initiated by addition of N_{α}-Benzoyl-L-arginine-4-nitroanilide-hydrochloride (L-BAPA, B3133, Merck) dissolved in dimethyl sulfoxide (DMSO) to 32 μM. Reactions were incubated at 37° C. with shaking at 400 RPM (double orbital shaking) and trypsin activity was monitored by measuring A₄₀₅ with a FLUOstar Omega plate-reader (BMG Labtech).

### Mass Spectrometry

Stocks of proteins in 50 mM Tris-HCl PH 8.0 with 100 mM NaCl were heated in a PCR machine to 75° C. for 10 min to aggregate contaminants. Aggregates were removed by centrifugation at 16,900 g at 4° C. for 30 min. The supernatant was diluted to 10 μM and acidified to 1% (v/v) with formic acid. Acidified proteins were analyzed on a Rapidfire Agilent 6550 quadrupole-time of flight mass spectrometer (Mass Spectrometry Research Facility, Department of Chemistry, University of Oxford) and spectra were deconvoluted using the Mass Hunter software platform (Agilent). Masses were predicted by ExPASy ProtParam, based on formation of all disulfide bonds and removal of N-terminal formylmethionine. Gluconylation is a spontaneous post-translational modification commonly found for His-tagged proteins expressed in E. coli, adding 178 to the mass.

### Differential Scanning Calorimetry (DSC)

DSC was performed on a MicroCal PEAQ-DSC (Malvern). 29 μM SBTI-His₆ was dialyzed into 50 mM Na₂HPO₄ adjusted to pH 2.0 or pH 7.4 with orthophosphoric acid. His₆-Thrombin site-SpyTag003-gastrobodies were dialyzed into 50 mM KH₂PO₄ (pH 2.0) or 50 mM K₂HPO₄ (pH 7.4). At a rate of 3° C./min at 3 atm pressure, thermal transitions were monitored from 20 to 110° C. Data were analyzed using MicroCal PEAQ-DSC analysis software (version 1.22). Blank buffer signal was subtracted from the experimental sample, followed by baseline subtraction. The observed transition was fitted to a two-state model to obtain the melting temperature (Tₘ) using MicroCal PEAQ-DSC analysis software (version 1.22).

### Protein Stability Prediction

SBTI (PDB ID: 1AVU) was modelled using Rosetta3 (Release Version 2018.09.60072). Missing density in the crystal structure (D125, D126, A140, E141, D142) was modelled using the remodel protocol. “Relax” was initially run for 5 iterations, to produce a starting structure for generating an ensemble of structures. The lowest energy structure from the first 5 iterations was used as input for running the relax protocol for 500 iterations (run 1). Root mean square deviation (RMSD) was plotted against Rosetta Energy Units (REU). The lowest energy structure from run 1 was relaxed another 500 times because of a lack of convergence in run 1. Structures within −503<REU<−497 and 0.119 Å<RMSD<0.238 Å were picked as the ensemble for pmutscan. Solvent-accessible surface area of residues of a representative structure in the ensemble was calculated using the Parameter Optimised Surfaces (POPS) webserver. Surface accessibility of residues was scored as the quotient of surface accessible area and surface area of isolated atoms (Q). Residues with Q>0.2 were deemed to be surface-accessible and included in pmutscan calculations. Surface-accessible residues (except cysteine) of all structures in the ensemble were mutated to all natural amino acids (mutations to introduce cysteine and proline were excluded) using pmutscan. Average AREU was calculated for each mutation and for each residue position. Excel (Microsoft) and PyMOL 2.3.4 (Schrödinger) were used to visualize data.

### Pepsin Digestion Assay

20 μM (alanine scan, FIG. 5B) or 3.75 μM (GT01, FIG. 7A) of the protein of interest was incubated with 1 mg/ml (final concentration 3,028 U/mL) pepsin from pig gastric mucosa (P6887, Merck) at 37° C. The stability of nanofitin, nanobody and SBTI (each at 20 μM) was compared in dilutions of 1 mg/mL pepsin in 50 mM glycine-HCl pH 2.2. Digestion was stopped by addition of SDS-loading buffer and heating for 3 min at 99° C. Samples were separated by SDS-PAGE, stained with Coomassie, and digestion was monitored by quantifying band intensity using ImageLab 6.0.1 (Bio-Rad). Assays were run in triplicate. Individual band intensity values were divided by mean band intensity of the corresponding protein at t=0 min and multiplied by 100 to set undigested samples (t=0 min) to 100%.

### Pancreatin Digestion Assay

7.5 μM SBTI, nanobody or nanofitin was incubated with 0, 0.1, 1 or 10 mg/mL pancreatin (from pig pancreas, P1750, Merck) in 50 mM Tris-HCl PH 6.8 with 10 mM CaCl₂) at 37° C. for 30 min. Digestion was stopped by heating in 1×SDS-loading buffer for 3 min at 99° C.

### Elastase Digestion Assay

7.5 μM SBTI, nanobody or nanofitin was incubated with 0, 0.1, 1 or 10 U/mL elastase (from pig pancreas, E7885, Merck) in 50 mM Tris-HCl PH 6.8 10 mM CaCl₂) at 37° C. for 30 min. Digestion was stopped by heating in 1×SDS-loading buffer for 3 min at 99° C.

### Temperature-Dependent Solubility Assay

Proteins were diluted to 30 μM in 50 mM Tris-HCl PH 8.0 with 100 mM NaCl and heated to 25, 75, 90 or 100° C. for 10 min. Aggregates were removed by centrifugation at 16,900 g at 4° C. for 30 min. The soluble fraction (supernatant) was separated by SDS-PAGE, stained with Coomassie, and band intensity was quantified using ImageLab 6.0.1 (Bio-Rad). The assay was performed in triplicate. Individual band intensity values were divided by mean band intensity of the corresponding mutant at 25° C. and multiplied by 100 to set samples kept at 25° C. to 100%.

### Phage Production and Purification

200 mL 2×TY with 2% (w/v) glucose+100 μg/mL ampicillin was inoculated from an overnight starter culture of E. coli TG1 (Lucigen) transformed with pBAD-DsbA (ss)-SBTI-pIII (216-425) (monoclonal WT or library) and grown at 37° C. at 200 RPM until OD₆₀₀ 0.5. Cultures were infected with the M13KO7 phage (New England Biolabs) at a multiplicity of infection of 10:1 (phage: bacteria) for 45 min at 37° C. at 80 RPM. Bacterial cells were pelleted by centrifugation at 2,500 g for 10 min at 4° C. and resuspended in 200 mL induction medium: 2×TY+0.2% (w/v) L-arabinose+100 μg/mL ampicillin+50 μg/mL kanamycin. Phage were grown overnight at 18° C. at 200 RPM.

Phage were precipitated by addition to 5% (w/v) polyethylene glycol 8000 (PEG8000, Thermo Fisher)+0.5 M NaCl for at least 1 h at 4° C., centrifuged at 15,000 g at 4° C., and the supernatant discarded. The phage pellet was resuspended in PBS and centrifuged at 15,000 g at 4° C. to remove bacterial cells. Precipitation was repeated for a total of three rounds. Purified phage were stored at −80° C. in PBS with 15% (v/v) glycerol. Phage stocks were titered in duplicate by quantitative PCR (qPCR) using primers Fwd2 (5′-GTCTGACCTGCCTCAACCTC-3′, SEQ ID NO: 60) and Rev2 (5′-TCACCGGAACCAGAGCCAC-3′, SEQ ID NO: 61) and 2× SensiMix (Bioline) master mix relative to a dilution series of M13KO7 (NEB). qPCR was performed on a Mx3000P qPCR machine (Agilent) and data were analyzed using MxPro qPCR software (Agilent).

### Phage Display Panning of Gastrobodies Against GTD

AviTag-His₆-GTD was biotinylated with GST-BirA. Excess biotin was removed by three dialysis steps, each for 3 h against PBS following by gel filtration as above. Selections were performed with a library of SBTI with five, six or seven randomized residues (NNK) in GDR1 and GDR2. Three rounds of selection were performed to obtain a first binder (GT_S1_01). In the first round, 10¹⁰ phage were incubated with 500 nM biotinylated AviTag-His₆-GTD in PBS+0.05% Tween-20 (PBS-T) with 1.5% (w/v) bovine serum albumin (BSA) for 2 h at 25° C., in a 96-well cell culture plate (655161, Greiner Bio) blocked with 3% (w/v) BSA. Phage bound to biotinylated bait were captured by incubating with Biotin Binder Dynabeads (Thermo Fisher) for 1 h at 25° C. Beads were washed four times at 25° C. with PBS-T, twice with 50 mM Tris-HCl PH 7.5+0.5 M NaCl and twice with PBS. Finally, phage were eluted with 0.1 M triethylamine pH 11.0 at 25° C., neutralized by adding 1 M Tris-HCl PH 7.4, and used to re-infect a log-phase culture of E. coli TG1 cells for amplification, as described above, for subsequent rounds of selection.

The second and third round of selection were performed with the following changes. A negative selection step was included before round 3 selection. Amplified phage from round 1 were incubated with Biotin Binder Dynabeads in PBS with 3% (w/v) BSA for 90 min at 25° C. Beads were settled by centrifugation for 1 min at 25° C. in a mini centrifuge (2,000 g). Unbound phage in the supernatant was used as phage input for subsequent selection. In round 2 and 3 phage input was reduced to 10⁸ particles. Bait concentration was reduced to 250 nM and three 10 min washes with PBS-T were added in round 2 and 3.

### Affinity Maturation Selections of Gastrobodies Against GTD

GT_S1_01 was used as starting clone for an affinity maturation library where each GDR was randomized separately. Each randomized GDR featured five, six or seven NNK codons. Selection was carried out as for the initial panning with the following modifications. Phage input was 10¹¹ (round 1 and 2) or 10¹⁰ (round 3). The concentration of biotinylated AviTag-His₆-GTD was reduced from 200 nM in round 1 to 100 nM (round 2) or 50 nM (round 3). Addition of excess non-biotinylated AviTag-His₆-GTD was used to drive off-rate selection and control the on-phase. In round 1, one 20 min off-rate wash at 25° C. with excess non-biotinylated bait was included. Two off-rate washes for 1 h (round 2) or 2 h (round 3) at 37° C. were performed.

Pepsin pressure was introduced in parallel to the standard selection after round 1 of affinity maturation. Amplified phage were incubated in 0.1 mg/mL pepsin in 50 mM glycine-HCl at pH 2.2 at 37° C. for 10 min. Digestion was stopped by addition of 2.5 M Tris pH 8.8. Phage were precipitated with 4% (w/v) PEG8000+0.5 M NaCl on ice at 4° C. for 1 h. Precipitated phage was pelleted by centrifugation at 16,900 g at 4° C. and the pellet was washed twice in ice-cold 4% (w/v) PEG8000+0.5 M NaCl, before resuspending in PBS with 1% (w/v) BSA.

### Phage Display Panning of Gastrobodies Against CROP

AviTag-His₆-CROP was biotinylated with GST-BirA. Excess biotin was removed by three dialysis steps, each for 3 h against PBS. Biotinylated AviTag-His₆-CROP was used as bait in selection experiments with a library of M13KO7-SBTI-pIII phage, where GDR1 (aa residues 22-25) and GDR2 (aa residues 47-50) of SBTI had been randomized with NNK codons. Two sets of three rounds of selection were performed. In the first round, 1013 phage were incubated with 0.5 μM biotinylated AviTag-His₆-CROP in 3% (w/v) BSA in PBS for 3 h at 25° C., in a microfuge tube blocked with 3% (w/v) BSA. Phage bound to biotinylated bait was captured by incubating with Biotin Binder Dynabeads (Thermo Fisher) for 1 h at 25° C. Beads were washed four times at 25° C. with PBS-T, once with 50 mM Tris-HCl pH 7.5+0.5 M NaCl, and twice with PBS. Finally, phage were eluted with 50 mM glycine-HCl pH 2.2 or 0.1 M triethylamine pH 11.0 at 25° C. The acid elution was neutralized with 2.5 M Tris-HCl PH 8.8, while the alkaline elution was neutralized with 1 M Tris-HCl pH 7.4. Neutralized eluted phage was used to re-infect a log-phase culture of TG1 cells for amplification, as described above, for subsequent rounds of selection.

The second and third rounds of selection were performed as for the first round with the following modifications. A negative selection step was included before round 2 and round 3 selection. SBTI-M13 phage were incubated with Biotin Binder Dynabeads in PBS with 3% (w/v) BSA for 90 min at 25° C. Beads were settled by centrifugation for 1 min at 25° C. in a mini centrifuge (2,000 g). Unbound phage in the supernatant were used as phage input for subsequent selection. Phage input was reduced to 1011 particles, bait concentration was reduced to 0.3 μM (round 2) or 0.2 μM (round 3), and incubation time of phage with bait was reduced to 1 h at 25° C. Two of the washes (one PBS-T and 50 mM Tris-HCl PH 7.5+0.5 M NaCl) in round 3 were incubated for 10 min at 25° C.

### Surface Plasmon Resonance (SPR)

SPR experiments were carried out using a Biacore T200 (Cytiva Lifesciences). The binding surface was created by flowing biotinylated AviTag-His₆-CROP or AviTag-His₆-GTD over a Sensor Chip CAP coated in Biotin CAPture reagent (Cytiva Lifesciences). Serial dilutions of analyte protein (His₆-Thrombin site-SpyTag003-GT01 or His₆-Thrombin Site-SpyTag003-(anti-CROP gastrobodies)) were injected in PBS+0.05% (v/v) Tween-20 at a flow rate of 60 μL/min for 200 s, followed by 200 s dissociation time. Triplicate dilution series of GT01 were analyzed. For anti-CROP gastrobodies a single dilution series was analyzed with one duplicate concentration. The binding surface was regenerated using 6 M guanidine-HCl+0.25 M NaOH. Measurements were performed at 25° C. with double referencing subtraction. Data were fitted to a 1:1 binding model using the Biacore T200 Evaluation software (Cytiva Lifesciences). For anti-GTD gastrobodies, kinetic analysis was used to obtain K_{off} (dissociation rate constant) and Kₒₙ (association rate constant). Anti-CROP gastrobodies were analyzed using equilibrium analysis to obtain K_{d} (dissociation constant).

### GTD Inhibition Assay

500 nM AviTag-His₆-GTD was incubated with serial dilutions of His₆-Thrombin site-SpyTag003-GT01 or His₆-Thrombin site-SpyTag003-SBTI in PBS for 15 min at 25° C. in a black 96-well half area no-binding plate (3993, Corning). The reaction was started by addition of UDP-Glucose to 25 μM and incubated for 1 h at 25° C. An equal volume of nucleotide detection reagent from the UDP-Glo glycosyltransferase assay kit (V6991, Promega) was added to stop the reaction, before continuing the incubation for 1 h at 25° C. UDP released in the glucosyltransferase reaction is converted into ATP by the nucleotide detection reagent. Bioluminescent signal is generated by a luciferase in the nucleotide detection reagent which requires ATP. Luminescence was recorded at 520 nm on a FLUOStar Omega plate-reader (BMG Labtech).

### Software

Data were analyzed and plotted in Microsoft Excel unless stated otherwise. MicroCal PEAQ-DSC analysis software (version 1.22) was used to analyze DSC data. MxPro qPCR software (Agilent) was used to analyze qPCR data. MARS (BMG Labtech) was used to analyze trypsin inhibition assay and gastric fluid proteolysis data. Gel images were analyzed in ImageLab (version 6.0.1, Bio-Rad). MS spectra were analyzed in Mass Hunter software platform (version B.07.00, Agilent).

1. A mutant Kunitz-type soybean trypsin inhibitor (SBTI) family polypeptide comprising two or more amino acid mutations compared to the corresponding unmutated (e.g. wild-type) SBTI family polypeptide, wherein the mutant SBTI family polypeptide comprises:
(i) one or more amino acid mutations in a first domain corresponding to positions 22-25 of SEQ ID NO: 1; and
(ii) one or more amino acid mutations in a second domain corresponding to positions 47-50 of SEQ ID NO: 1,
wherein the mutant SBTI family polypeptide:
(a) binds selectively to a ligand that does not bind to the corresponding unmutated (e.g. wild-type) SBTI family polypeptide; and
(b) is resistant to cleavage by pepsin.

2. The mutant SBTI family polypeptide of claim 1, wherein the unmutated SBTI family polypeptide is a serine protease inhibitor, preferably a trypsin and/or chymotrypsin inhibitor.

3. The mutant SBTI family polypeptide of claim 2, wherein the unmutated SBTI family polypeptide is selected from the list consisting of:
(A) (i) a polypeptide comprising an amino acid sequence as set forth in SEQ ID NO: 1, 12, 13 or 62 (e.g. SBTI, Uniprot ID P01070);
(ii) a polypeptide comprising an amino acid sequence as set forth in SEQ ID NO: 2 (e.g. WBA, Uniprot ID P15465);
(iii) a polypeptide comprising an amino acid sequence as set forth in SEQ ID NO: 3 (e.g. ECTI, Uniprot ID P09943);
(iv) a polypeptide comprising an amino acid sequence as set forth in SEQ ID NO: 4 (e.g. WCI, Uniprot ID P10822);
(v) a polypeptide comprising an amino acid sequence as set forth in SEQ ID NO: 5 (e.g. CATI, Uniprot ID Q9M3Z7);
(vi) a polypeptide comprising an amino acid sequence as set forth in SEQ ID NO: 6 (e.g. EnCTI, Uniprot ID P86451);
(vii) a polypeptide comprising an amino acid sequence as set forth in SEQ ID NO: 7 (e.g. DRTI, Uniprot ID P83667);
(viii) a polypeptide comprising an amino acid sequence as set forth in SEQ ID NO: 8 (e.g. SOTI, Uniprot ID A0A097P6E1);
(ix) a polypeptide comprising an amino acid sequence as set forth in SEQ ID NO: 9 (e.g. BBTI, Uniprot ID Q6VEQ7);
(x) a polypeptide comprising an amino acid sequence as set forth in SEQ ID NO: 10 (e.g. AMTI, Uniprot ID P35812);
(xi) a polypeptide comprising an amino acid sequence as set forth in SEQ ID NO: 11 (e.g. SSTI, Uniprot ID Q7M1P4); and
(xii) a polypeptide comprising an amino acid sequence having at least 80% (e.g. 85%, 90% or 95%) sequence identity to an amino acid sequence of any one of SEQ ID NOs: 1-13 or 62, preferably wherein the polypeptide is a wild-type polypeptide; and/or
(B) the mutant SBTI family polypeptide comprises a mutation that eliminates or reduces its serine proteinase inhibitory activity, preferably its trypsin and/or chymotrypsin inhibitory activity.

4. (canceled)

5. The mutant SBTI family polypeptide of claim 1, wherein;
(i) the first domain comprises two or more amino acid mutations;
(ii) the second domain comprises two or more amino acid mutations;
(iii) the first domain comprises two or more amino acid substitutions and/or insertions and/or the second domain comprises two or more amino acid substitutions and/or insertions;
(iv) all of the amino acids in the first domain are substituted;
(v) all of the amino acids in the second domain are substituted;
(vi) the first domain contains 1-15 amino acid insertions, optionally 1-10 amino acid insertions (e.g. 1-6 amino acid insertions); and/or
(vii) the second domain contains 1-15 amino acid insertions, optionally 1-10 amino acid insertions (e.g. 1-6 amino acid insertions).

6-11. (canceled)

12. The mutant SBTI family polypeptide of claim 1, wherein mutant SBTI family polypeptide further comprises:
(i) one or more amino acid mutations in a domain corresponding to positions 6-9 of SEQ ID NO: 1;
(ii) one or more amino acid mutations in a domain corresponding to positions 36-38 of SEQ ID NO: 1;
(iii) one or more amino acid mutations in a domain corresponding to positions 63-65 of SEQ ID NO: 1;
(iv) one or more amino acid mutations in a domain corresponding to positions 84-87 of SEQ ID NO: 1; and/or
(v) one or more amino acid mutations in a domain corresponding to positions 124-128 of SEQ ID NO: 1.

13. (canceled)

14. The mutant SBTI family polypeptide of claim 1, wherein the mutant SBTI family polypeptide comprises an amino acid sequence with at least 70% (e.g. 75%, 80%, 85% or 90%) sequence identity to an amino acid sequence of any one of SEQ ID NOs: 1-13 or 62.

15. The mutant SBTI family polypeptide of claim 1, wherein the ligand that does not bind to the corresponding unmutated (e.g. wild-type) SBTI family polypeptide is:
(i) a gastrointestinal (GI) tract ligand;
(ii) a gastrointestinal (GI) tract ligand associated with a disease or condition of the GI tract;
(iii) a gastrointestinal (GI) tract ligand associated with a disease or condition of the GI tract that is caused by a pathogen;
(iv) a molecule associated with a pathogen;
(v) a molecule on the surface of a pathogen or a toxin produced by a pathogen; or
(vi) a polypeptide, a peptide, a polysaccharide or a small molecule toxin.

16-18. (canceled)

19. The mutant SBTI family polypeptide of claim 15, wherein:
(i) the pathogen is a bacterium, virus or protozoa; or
(ii) the disease or condition of the GI tract is an inflammatory disease or condition (such as inflammatory bowel disease) or neoplastic disease or condition (such as a GI tract cancer).

20-21. (canceled)

22. The mutant SBTI family polypeptide of claim 1, wherein the mutant SBTI family polypeptide is:
(i) conjugated to another molecule;
(ii) conjugated to a therapeutic agent, an enzyme or a signal generating agent; or
(iii) part of a fusion protein.

23. (canceled)

24. A nucleic acid molecule encoding the mutant SBTI family polypeptide of claim 1.

25. A composition comprising the mutant SBTI family polypeptide of claim 1, optionally wherein the composition is a pharmaceutical composition (optionally formulated for oral administration), an animal feed, nutraceutical, dietary supplement or medical food.

26-28. (canceled)

29. A library of nucleic acid molecules encoding a plurality of mutant SBTI family polypeptides each comprising two or more amino acid mutations compared to their corresponding unmutated (e.g. wild-type) SBTI family polypeptides, wherein each mutant SBTI family polypeptide comprises:
(i) one or more amino acid mutations in a first domain corresponding to positions 22-25 of SEQ ID NO: 1; and
(ii) one or more amino acid mutations in a second domain corresponding to positions 47-50 of SEQ ID NO: 1.

30. The library of nucleic acid molecules of claim 29, wherein:
(1) the unmutated SBTI family polypeptide is:
(A) a serine protease inhibitor, preferably a trypsin and/or chymotrypsin inhibitor; and/or
(B) selected from the list consisting of:
(i) a polypeptide comprising an amino acid sequence as set forth in SEQ ID NO: 1, 12, 13 or 62 (e.g. SBTI, Uniprot ID P01070);
(ii) a polypeptide comprising an amino acid sequence as set forth in SEQ ID NO: 2 (e.g. WBA, Uniprot ID P15465);
(iii) a polypeptide comprising an amino acid sequence as set forth in SEQ ID NO: 3 (e.g. ECTI, Uniprot ID P09943);
(iv) a polypeptide comprising an amino acid sequence as set forth in SEQ ID NO: 4 (e.g. WCI, Uniprot ID P10822);
(v) a polypeptide comprising an amino acid sequence as set forth in SEQ ID NO: 5 (e.g. CATI, Uniprot ID Q9M3Z7);
(vi) a polypeptide comprising an amino acid sequence as set forth in SEQ ID NO: 6 (e.g. EnCTI, Uniprot ID P86451);
(vii) a polypeptide comprising an amino acid sequence as set forth in SEQ ID NO: 7 (e.g. DRTI, Uniprot ID P83667);
(viii) a polypeptide comprising an amino acid sequence as set forth in SEQ ID NO: 8 (e.g. SOTI, Uniprot ID A0A097P6E1);
(ix) a polypeptide comprising an amino acid sequence as set forth in SEQ ID NO: 9 (e.g. BBTI, Uniprot ID Q6VEQ7);
(x) a polypeptide comprising an amino acid sequence as set forth in SEQ ID NO: 10 (e.g. AMTI, Uniprot ID P35812);
(xi) a polypeptide comprising an amino acid sequence as set forth in SEQ ID NO: 11 (e.g. SSTI, Uniprot ID Q7M1P4); and
(xii) a polypeptide comprising an amino acid sequence having at least 80% (e.g. 85%, 90% or 95%) sequence identity to an amino acid sequence of any one of SEQ ID NOs: 1-13 or 62, preferably wherein the polypeptide is a wild-type polypeptide; and/or
(2) the mutant SBTI family polypeptide is a mutant SBTI family polypeptide, wherein:
(i) the first domain comprises two or more amino acid mutations;
(ii) the second domain comprises two or more amino acid mutations;
(iii) the first domain comprises two or more amino acid substitutions and/or insertions and/or the second domain comprises two or more amino acid substitutions and/or insertions;
(iv) all of the amino acids in the first domain are substituted;
(v) all of the amino acids in the second domain are substituted;
(vi) the first domain contains 1-15 amino acid insertions, optionally 1-10 amino acid insertions (e.g. 1-6 amino acid insertions); and/or
(vii) the second domain contains 1-15 amino acid insertions, optionally 1-10 amino acid insertions (e.g. 1-6 amino acid insertions).

31. The library of nucleic acid molecules of claim 29, wherein the library of nucleic acid molecules encodes a phage display library, an mRNA display library, a bacterial display library, a yeast display library or a ribosome display library.

32. A plurality of mutant SBTI family polypeptides encoded by the library of nucleic acid molecules of claim 29.

33. The plurality of mutant SBTI family polypeptides of claim 32, wherein the polypeptides are displayed on phage particles.

34-36. (canceled)

37. A method of identifying a mutant SBTI family polypeptide that binds selectively to a ligand of interest (e.g. a ligand that does not bind to the corresponding unmutated (e.g. wild-type) SBTI family polypeptide) comprising:
(i) providing a plurality of mutant SBTI family polypeptides as defined in claim 32;
(ii) contacting the plurality of mutant SBTI family polypeptides of (i) with the ligand of interest; and
(iii) isolating a mutant SBTI family polypeptide that binds selectively to the ligand of interest thereby identifying a mutant SBTI family polypeptide that binds selectively to the ligand of interest.

38. The method of claim 37, wherein:
(1) the ligand of interest is a ligand that does not bind to the corresponding unmutated (e.g. wild-type) SBTI family polypeptide and is:
(a) a gastrointestinal (GI) tract ligand;
(b) a gastrointestinal (GI) tract ligand associated with a disease or condition of the GI tract;
(c) a gastrointestinal (GI) tract ligand associated with a disease or condition of the GI tract that is caused by a pathogen;
(d) a molecule associated with a pathogen;
(e) a molecule on the surface of a pathogen or a toxin produced by a pathogen; or
(f) a polypeptide, a peptide, a polysaccharide or a small molecule toxin; and/or
(2) step (iii) is performed by a method selected from phage display, mRNA display, bacterial display, yeast display or ribosome display.

39. (canceled)

40. A method of identifying a mutant SBTI family polypeptide that binds selectively to a region of interest of the gastrointestinal tract of an animal comprising:
(i) administering a plurality of mutant SBTI family polypeptides as defined in claim 32 to the gastrointestinal tract of an animal (e.g. orally);
(ii) isolating a mutant SBTI family polypeptide (e.g. a phage particle displaying a mutant SBTI family polypeptide) that is non-covalently bound to the region of interest of gastrointestinal tract of the animal; and
(iii) identifying the mutant SBTI family polypeptide isolated in step (ii).

41. The method of claim 40 further comprising a step of identifying the ligand to which the mutant SBTI family polypeptide binds.


SEQUENCE LISTING (86 sequences)

SEQ ID NO: 1 - protein, 181 aa, Glycine max
DFVLDNEGNPLENGGTYYILSDITAFGGIRAAPTGNERCPLTVVQSRNELDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 2 - protein, 175 aa, Psophocarpus tetragonolobus
ADDPVYDAEGNKLVNRGKYTIVSFSDGAGIDVVATGNENPEDPLSIVKSTRNIMYATSISSEDKTPPQPRNILENMRLKINFATDPHKGDVWSVVDFQPDGQQLKLAGRYPNQVKGAFTIQKGSNTPRTYKLLFCPVGSPCKNIGISTDPEGKKRLVVSYQSDPLVVKFHRHEPE

SEQ ID NO: 3 - protein, 172 aa, Erythrina caffra
VLLDGNGEVVQNGGTYYLLPQVWAQGGGVQLAKTGEETCPLTVVQSPNELSDGKPIRIESRLRSAFIPDDDKVRIGFAYAPKCAPSPWWTVVEDEQEGLSVKLSEDESTQFDYPFKFEQVSDQLHSYKLLYCEGKHEKCASIGINRDQKGYRRLVVTEDYPLTVVLKKDESS

SEQ ID NO: 4 - protein, 183 aa, Psophocarpus tetragonolobus
DDDLVDAEGNLVEGGGTYYLLPHIWAHGGGIETAKTGNEPCPLTVVRSPNEVSKGEPIRISSQFLSLFIPRGSLVALGFANPPSCAASPWWTVVDSPQGPAVKLSQQKLPEKDILVFKFEKVSHSNIHVYKLLYCQHDEEDVKCDQYIGIHRDRNGNRRLVVTEENPLELVLLKAKSETASSH

SEQ ID NO: 5 - protein, 194 aa, Cicer arietinum
FSNEDVEQVLDINGNPIFPGGKYYILPAIRGPPGGGVRLDKTGDSECPVTVLQDYKEVINGLPVKFVIPGISPGIIFTGTPIEIEFTKKPNCAESSKWLIFVDDTIDKACIGIGGPENYSGKQTLSGTFNIQKYGSGFGYKLGFCVKGSPICLDIGRYDNDEGGRRLNLTEHEAFRVVFVDASSYEDGIVKSVV

SEQ ID NO: 6 - protein, 176 aa, Enterolobium contortisiliquum
KELLDSDGDILRNGGTYYILPALRGKGGGLELAKTGDETCPLNVVQARSETKRGRPAIIWTPPRIAILTPAFYLNIEFQTRDLPACLEEYSRLPWKVEGESQEVKIAPKEEEQHLFGSFKIKPYRDDYKLVYCEGNSDDDSCKDLGISIDDENNRLLVVKDGDPLAVRFVKAHRRG

SEQ ID NO: 7 - protein, 185 aa, Delonix regia [sequence omitted]

SEQ ID NO: 8 - protein, 182 aa, Senna obtusifolia
IVFDSDGDFLRNGGTYMLSPPNGGGGILAAAIKQGSDRDCSLGVIQHESYTGWPVTISALVRPTFISTSFQLLLSFAYIPPNVCTKNSDWIIKSSNDFEGTVMLGDDKNPVGSLFFIKSYDSSKNYYKLVVCGGRGDEHCRNIGVDKDENGYKRLVVTEGEPLVLQFDKVNKGNFAFESNLS

SEQ ID NO: 9 - protein, 175 aa, Bauhinia bauhinioides
SSVVVDTNGQPVSNGADAYYLVPVSHGHAGLALAKIGNEAEPRAVVLDPHHRPGLPVRFESPLRINIIKESYFLNIKFGPSSSDSGVWDVIQQDPIGLAVKVTDTKSLLGPFKVEKEGEGYKIVYYPERGQTGLDIGLVHRNDKYYLAVKDGEPCVFKIRKATDEESFAGIMSIV

SEQ ID NO: 10 - protein, 184 aa, Alocasia macrorrhiza
TNPVLDVDGNELQRGQLYYATSVMRPGGGLTLAAPKGSCPLNVAQAPFDEYSGRPLAFFPENADDDTVQEGSTLYIMFPEPTRCPQSTVWTFDREAGFVTTGGTTSKAIGPHNSRFAIRKAGDASSQPRDYQIEVCPCSTGVERPSCRMGCLGTLGLAEGGKNVLLNINNESPHTIRFVKVKEG

SEQ ID NO: 11 - protein, 176 aa, Sagittaria sagittifolia
PVVDSDGDAVQLNLGGNYPLYTIQSAAIGFRGGLSTLRKDACKSYVYEAPETDRGLPVGFSASATSQPVMQLGSRYKFSFSMPVPLICDTAWSIGKSETNGGISFQPITAGDYFYLNNFSWFEARSTEETGVYKLAACSCEFCKIACPEVGSFNVNGRTLLGIGGEHFTVQFQKFD

SEQ ID NO: 12 - protein, 181 aa, Glycine max
DFVLDNEGNPLSNGGTYYILSDITAFGGIRAAPTGNERCPLTVVQSRNELDKGIGTIISSPFRIRFIAEGNPLRLKFDSFAVIMLCVGIPTEWSVVEDLPEGPAVKIGENKDAVDGWFRIERVSDDEFNNYKLVFCTQQAEDDKCGDIGISIDHDDGTRRLVVSKNKPLVVQFQKVDKESL

SEQ ID NO: 13 - protein, 181 aa, Glycine max
DFVLDNEGNPLENGGTYYILSDITAFGGIRAAPTGNERCPLTVVQSRNELDKGIETIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 14 - protein, 6 aa, Artificial sequence
  SBTI Beta strand 1
GTYYIL

SEQ ID NO: 15 - protein, 4 aa, Artificial sequence
  SBTI Beta strand 2
IRAA

SEQ ID NO: 16 - protein, 4 aa, Artificial sequence
  SBTI Beta strand 3
TVVQ

SEQ ID NO: 17 - protein, 4 aa, Artificial sequence
  SBTI Beta strand 4
TIIS

SEQ ID NO: 18 - protein, 5 aa, Artificial sequence
  SBTI Beta strand 5
LSLKF

SEQ ID NO: 19 - protein, 3 aa, Artificial sequence
  SBTI Beta strand 6
SVV

SEQ ID NO: 20 - protein, 3 aa, Artificial sequence
  SBTI Beta strand 7
AVK

SEQ ID NO: 21 - protein, 7 aa, Artificial sequence
  SBTI Beta strand 8
GWFRLER

SEQ ID NO: 22 - protein, 7 aa, Artificial sequence
  SBTI Beta strand 9
YKLVFCP

SEQ ID NO: 23 - protein, 5 aa, Artificial sequence
  SBTI Beta strand 10
IGISI

SEQ ID NO: 24 - protein, 5 aa, Artificial sequence
  SBTI Beta strand 11
RRLVV

SEQ ID NO: 25 - protein, 5 aa, Artificial sequence
  SBTI Beta strand 12
VQFQK

SEQ ID NO: 26 - protein, 4 aa, Artificial sequence
  SBTI 1st domain
DITA

SEQ ID NO: 27 - protein, 4 aa, Artificial sequence
  SBTI 2nd domain
RNEL

SEQ ID NO: 28 - protein, 4 aa, Artificial sequence
  GDR1
IAGT

SEQ ID NO: 29 - protein, 4 aa, Artificial sequence
  GDR2
LVFP

SEQ ID NO: 30 - protein, 4 aa, Artificial sequence
  GDR1
HPDL

SEQ ID NO: 31 - protein, 4 aa, Artificial sequence
  GDR2
LYLF

SEQ ID NO: 32 - protein, 4 aa, Artificial sequence
  GDR1
  VARIANT (2)..(2): X is preferably Q
LXNR

SEQ ID NO: 33 - protein, 4 aa, Artificial sequence
  GDR2
HDFM

SEQ ID NO: 34 - protein, 4 aa, Artificial sequence
  GDR1
PYRY

SEQ ID NO: 35 - protein, 4 aa, Artificial sequence
  GDR2
WLHR

SEQ ID NO: 36 - protein, 4 aa, Artificial sequence
  GDR1
  VARIANT (4)..(4): X is preferably Q
VPHX

SEQ ID NO: 37 - protein, 4 aa, Artificial sequence
  GDR2
RNKW

SEQ ID NO: 38 - protein, 4 aa, Artificial sequence
  GDR1
SFRG

SEQ ID NO: 39 - protein, 4 aa, Artificial sequence
  GDR2
FRLE

SEQ ID NO: 40 - protein, 4 aa, Artificial sequence
  GDR1
RPPQ

SEQ ID NO: 41 - protein, 4 aa, Artificial sequence
  GDR2
TWGW

SEQ ID NO: 42 - protein, 6 aa, Artificial sequence
  GDR1
DYGRQL

SEQ ID NO: 43 - protein, 6 aa, Artificial sequence
  GDR2
LFRNHR

SEQ ID NO: 44 - protein, 181 aa, Artificial sequence
  G9
DFVLDNEGNPLENGGTYYILSHPDLFGGIRAAPTGNERCPLTVVQSLYLFDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 45 - protein, 181 aa, Artificial sequence
  G22
DFVLDNEGNPLENGGTYYILSLQNRFGGIRAAPTGNERCPLTVVQSHDFMDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 46 - protein, 181 aa, Artificial sequence
  T12
DFVLDNEGNPLENGGTYYILSIAGTFGGIRAAPTGNERCPLTVVQSLVFPDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 47 - protein, 181 aa, Artificial sequence
  T42
DFVLDNEGNPLENGGTYYILSVPHQFGGIRAAPTGNERCPLTVVQSRNKWDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 48 - protein, 181 aa, Artificial sequence
  G28
DFVLDNEGNPLENGGTYYILSPYRYFGGIRAAPTGNERCPLTVVQSWLHRDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 49 - protein, 181 aa, Artificial sequence
  T23
DFVLDNEGNPLENGGTYYILSSFRGFGGIRAAPTGNERCPLTVVQSFRLEDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 50 - protein, 181 aa, Artificial sequence
  T29
DFVLDNEGNPLENGGTYYILSRPPQFGGIRAAPTGNERCPLTVVQSTWGWDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 51 - protein, 185 aa, Artificial sequence
  GT01
DFVLDNEGNPLENGGTYYILSDYGRQLFGGIRAAPTGNERCPLTVVQSLFRNHRDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 52 - DNA, 543 bp, Artificial sequence
  G9
gattttgttcttgataacgagggtaaccctcttgaaaacggcggtacttactatattttgtcgcatcctgatctttttgggggcatccgtgcagcaccgacaggtaacgaacgctgtcctcttaccgtagttcagtctctgtatttgtttgataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcagcgatgacgagttcaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 53 - DNA, 543 bp, Artificial sequence
  G22
gattttgttcttgataacgagggtaaccctcttgaaaacggcggtacttactatattttgtcgcttcagaatcggtttgggggcatccgtgcagcaccgacaggtaacgaacgctgtcctcttaccgtagttcagtctcatgattttatggataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcagcgatgacgagttcaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 54 - DNA, 543 bp, Artificial sequence
  T12
gattttgttcttgataacgagggtaaccctcttgaaaacggcggtacttactatattttgtcgattgctgggacgtttgggggcatccgtgcagcaccgacaggtaacgaacgctgtcctcttaccgtagttcagtctcttgtttttcctgataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcagcgatgacgagttcaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 55 - DNA, 543 bp, Artificial sequence
  T42
gattttgttcttgataacgagggtaaccctcttgaaaacggcggtacttactatattttgtcggtgcctcatcagtttgggggcatccgtgcagcaccgacaggtaacgaacgctgtcctcttaccgtagttcagtctcgtaataagtgggataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcagcgatgacgagttcaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 56 - DNA, 543 bp, Artificial sequence
  G28
gattttgttcttgataacgagggtaaccctcttgaaaacggcggtacttactatattttgtcgccgtatcgttattttgggggcatccgtgcagcaccgacaggtaacgaacgctgtcctcttaccgtagttcagtcttggcttcatcgggataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcagcgatgacgagttcaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 57 - DNA, 543 bp, Artificial sequence
  T23
gattttgttcttgataacgagggtaaccctcttgaaaacggcggtacttactatattttgtcgagttttcgtgggtttgggggcatccgtgcagcaccgacaggtaacgaacgctgtcctcttaccgtagttcagtcttttaggctggaggataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcagcgatgacgagttcaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 58 - DNA, 543 bp, Artificial sequence
  T29
gattttgttcttgataacgagggtaaccctcttgaaaacggcggtacttactatattttgtcgcggccgcctcagtttgggggcatccgtgcagcaccgacaggtaacgaacgctgtcctcttaccgtagttcagtctacttgggggtgggataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcagcgatgacgagttcaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 59 - DNA, 555 bp, Artificial sequence
  GT01
gattttgttcttgataacgagggtaaccctcttgaaaacggcggtacttactatattttgtcggattatgggaggcagctttttgggggcatccgtgcagcaccgacaggtaacgaacgctgtcctcttaccgtagttcagtctctgtttcgtaatcatcgtgataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcagcgatgacgagttcaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 60 - DNA, 20 bp, Artificial sequence
  phage primer Fwd2
gtctgacctgcctcaacctc

SEQ ID NO: 61 - DNA, 19 bp, Artificial sequence
  phage primer Rev2
tcaccggaaccagagccac

SEQ ID NO: 62 - protein, 192 aa, Glycine max
DFVLDNEGNPLENGGTYYILSDITAFGGIRAAPTGNERCPLTVVQSRNELDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESLAKKNHGLSRSE

SEQ ID NO: 63 - protein, 4 aa, Artificial sequence
  GDR3
NEGN

SEQ ID NO: 64 - protein, 5 aa, Artificial sequence
  GDR4
SDDEF

SEQ ID NO: 65 - protein, 5 aa, Artificial sequence
  GDR4
RKTRA

SEQ ID NO: 66 - protein, 4 aa, Artificial sequence
  GDR3
TSGR

SEQ ID NO: 67 - protein, 9 aa, Artificial sequence
  GDR4
SHGRLRTVA

SEQ ID NO: 68 - protein, 4 aa, Artificial sequence
  GDR3
VDGS

SEQ ID NO: 69 - protein, 7 aa, Artificial sequence
  GDR4
WPTNTGF

SEQ ID NO: 70 - protein, 5 aa, Artificial sequence
  GDR4
NPSNG

SEQ ID NO: 71 - protein, 4 aa, Artificial sequence
  GDR3
DQGR

SEQ ID NO: 72 - protein, 7 aa, Artificial sequence
  GDR4
NTLWGSR

SEQ ID NO: 73 - protein, 4 aa, Artificial sequence
  GDR3
MDGA

SEQ ID NO: 74 - protein, 5 aa, Artificial sequence
  GDR4
DVNFQ

SEQ ID NO: 75 - protein, 4 aa, Artificial sequence
  GDR3
TQGG

SEQ ID NO: 76 - protein, 7 aa, Artificial sequence
  GDR4
RPTSSTG

SEQ ID NO: 77 - protein, 4 aa, Artificial sequence
  GDR3
IKGR

SEQ ID NO: 78 - protein, 5 aa, Artificial sequence
  GDR4
PTGAG

SEQ ID NO: 79 - protein, 4 aa, Artificial sequence
  GDR3
TGGR

SEQ ID NO: 80 - protein, 5 aa, Artificial sequence
  GDR4
RRRDR

SEQ ID NO: 81 - protein, 185 aa, Artificial sequence
  GT44
DFVLDNEGNPLENGGTYYILSDYGRQLFGGIRAAPTGNERCPLTVVQSLFRNHRDKGIGTIISSPYRIRFIAEGHPLSLKFDSFAVIMLCVGIPTEWSVVEDLPEGPAVKIGENKDAMDGWFRLERVNPSNGNNYKLVFCPQQAEDDKCGDIGISIDHDDGTRRLVVSKNKPLVVQFQKLDKESL

SEQ ID NO: 82 - protein, 187 aa, Artificial sequence
  GT47
DFVLDNEGNPLENGGTYYILSDYGRQLFGGIRAAPTGNERCPLTVVQSLFRNHRDKGIGTIISSPYRIRFIAEGHPLSLKFDSFAVIMLCVGIPTEWSVVEDLPEGPAVKIGENKDAMDGWFRLERVRPTSSTGNNYKLVFCPQQAEDDKCGDIGISIDHDDGTRRLVVSKNKPLVVQFQKLDKESL

SEQ ID NO: 83 - DNA, 555 bp, Artificial sequence
  GT44
gattttgttcttgataacgagggtaaccctcttgaaaacggcggtacttactatattttgtcggattatgggaggcagctttttgggggcatccgtgcagcaccgacaggtaacgaacgctgtcctcttaccgtagttcagtctctgtttcgtaatcatcgtgataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtcaatcctagtaatggtaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 84 - DNA, 561 bp, Artificial sequence
  GT47
gattttgttcttgataacgagggtaaccctcttgaaaacggcggtacttactatattttgtcggattatgggaggcagctttttgggggcatccgtgcagcaccgacaggtaacgaacgctgtcctcttaccgtagttcagtctctgtttcgtaatcatcgtgataaggggatcggcactattatcagtagcccataccgtatccgttttattgccgagggtcatcctttatccttgaaattcgactcctttgcagtcatcatgttatgcgtcggaatcccaacagagtggagtgtagtagaagatttgccagagggtcctgcagtaaaaattggggagaataaagatgcaatggatgggtggttccgcttggagcgtgtccgtccgacttcgtctacggggaacaattacaaactggtgttctgccctcagcaggcggaggacgacaagtgtggagacatcggcatctccatcgaccatgacgatgggacgcgccgcttagtcgtcagcaaaaataaacctttagtagtccaatttcagaagcttgacaaggaatctttg

SEQ ID NO: 85 - protein, 163 aa, Artificial sequence
  BBKICA
VVVDTNGQPVSNGADAYYLVPVSHGHAGLALAKIGNEAEPRAVVLDPHHRPGLPVRFESPLRINIIKESYFLNIKFGPSSSDSGVWDVIQQDPIGLAVKVTDTKSLLGPFKVEKEGEGYKIVYYPERGQTGLDIGLVHRNDKYYLAVKDGEPAVFKIRKATDE

SEQ ID NO: 86 - DNA, 489 bp, Artificial sequence
  BBKICA
gtggtcgtagatacaaatggccagccagtaagtaatggggctgatgcttattatttagtcccagttagccatggacacgctggcttggccttagcgaaaattggcaacgaagctgaaccacgcgcagtggttcttgatccacaccaccgcccaggtttaccagttcgtttcgagtctccgcttcgtatcaacatcattaaggagtcttactttttaaacatcaaatttggtccatcaagtagcgattcaggagtatgggacgttatccagcaagacccgattgggttagcggtaaaggttaccgacacaaagagtctgttaggaccttttaaagtagaaaaagaaggtgaggggtacaagattgtttattatcccgagcgcgggcaaacagggttggacatcggattagtccaccgcaatgataaatattacctggctgtcaaagacggtgagccggcggtgttcaagatccgcaaagctaccgatgag